(19) United States
(12) Patent Application Publication
(10) Pub. No.: US 2025/0188542 A1
(43) Pub. Date: Jun. 12, 2025

(54) ENRICHMENT AND CHARACTERIZATION OF RARE CIRCULATING CELLS, INCLUDING PROGENITOR CELLS, FROM PERIPHERAL BLOOD, AND USES THEREOF

(71) Applicants: Cornell University, Ithaca, NY (US); Hospital for Special Surgery, New York, NY (US)

(72) Inventors: Steven Zvi JOSEFOWICZ, New York, NY (US); Jin Gyu CHEONG, New York, NY (US); Franck BARRAT, New York, NY (US)

(73) Assignees: Cornell University, Ithaca, NY (US); Hospital for Special Surgery, New York, NY (US)

(21) Appl. No.: 18/834,832

(22) PCT Filed: Feb. 6, 2023
(86) PCT No.: PCT/US2023/062066

§ 371 Date: Jul. 31, 2024

Related U.S. Application Data
(60) Provisional application No. 63/306,956, filed on Feb. 4, 2022.

(51) Int. Cl. C12Q 1/6883; C12Q 1/6874; G01N 33/68; G16B 30/00

(52) U.S. Cl. CPC C12Q 1/6883; C12Q 1/6874; G01N 33/6875; G16B 30/00; C12Q 2600/156; C12Q 2600/158

(57) ABSTRACT

The present disclosure encompasses systems, methods, and compositions for enriching a population of rare circulating cells, including progenitor cells, from peripheral blood. Specific embodiments encompass methods of analyzing rare circulating cell transcriptomic, genetic, protein expression, metabolic, epigenomic, and/or other functional assay data to identify differential gene or protein expression and/or chromatin accessibility, and/or functional characteristics. Particular methods relate to enriching and analyzing rare circulating cells in patients following COVID-19 infection, and treating the patient based on the analysis. Embodiments also relate to an enriched population of rare circulating cells from peripheral blood and uses thereof.

### CROSS REFERENCE TO RELATED APPLICATION

The present application is national phase under 35 U.S.C. § 371 from PCT/US2023/062066, filed Feb. 6, 2023, which claims the benefit of priority under 35 U.S.C. § 119(e) to U.S. Provisional Application No. 63/306,956, DURABLE ALTERATIONS OF HEMATOPOIESIS AND CHROMATIN POST-COVID-19, filed on Feb. 4, 2022, which is currently co-pending herewith and which is incorporated by reference in its entirety.

### STATEMENT REGARDING FEDERALLY-SPONSORED RESEARCH

This invention was made with government support under Grant Nos. R01A|148416-0351 and R01A|148416-S2 awarded by the National Institutes of Health (NIH). The government has certain rights in the invention.

### FIELD

The present disclosure relates generally to the field of immunology, and particularly relates to systems and methods for obtaining and using blood stem cells to diagnose and treat disease.

### BACKGROUND

The embodiments disclosed herein are generally directed towards systems, software and methods for generating insights related to hematopoietic stem and progenitor cells (HSPC) and innate immune cell epigenetic alterations in human health and disease.

Stem cells are generally obtained as bone marrow stem cells. Because bone marrow aspiration and biopsy are lengthy, complicated, and costly processes, there is a need for methods and systems for obtaining stem cells from sources other than bone marrow. Obtaining stem cells from a more accessible source, such as from blood, would be advantageous from both a cost and patient experience perspective.

Further, the involvement of blood stem cells in health and diverse diseases is extensive and untapped. There is a need for methods and systems for non-invasive acquisition of stem cells to discover and measure their disease states, and measure reversal of these disease states with novel therapies or determine the efficacy of existing ones.

For example, acute SARS-CoV-2 infection is often highly inflammatory and protracted. Recent advances have established that inflammation can trigger innate immune memory and a persistent influence on hematopoietic development, through epigenetic mechanisms. However, these phenotypes and their molecular and cellular features are poorly described in humans. Hence there is a need to characterize and in turn better understand these phenotypes and their molecular and cellular features.

### SUMMARY OF THE INVENTION

Embodiments of the invention relate to methods of characterizing cellular molecular features and/or functional characteristics in an enriched population of rare circulating cells from peripheral blood, the method including: isolating one or more types of rare circulating cells from peripheral blood or from peripheral blood mononuclear cells (PBMC) from a peripheral blood sample; enriching the one or more types of rare circulating cells in the PBMC and/or in the peripheral blood sample, thereby providing an enriched population of rare circulating cells from the peripheral blood and/or PBMC; acquiring single cell and/or bulk transcriptomic, genetic, and/or protein expression, and/or metabolic, epigenomic, and/or other functional assay data for the enriched population of rare circulating cells; analyzing the enriched rare circulating cell transcriptomic, genetic, and/or protein expression, and/or metabolic, epigenomic, and/or other functional assay data to identify cellular molecular features and/or functional characteristics; and generating an output including transcriptional, genetic, protein, metabolic, epigenomic, and/or functional characteristic signatures, thereby characterizing cellular molecular features and/or functional characteristics for the one or more types of rare circulating cells.

Embodiments of the invention also encompass methods of enriching rare circulating cells from peripheral blood, wherein the method includes: isolating one or more types of rare circulating cells from peripheral blood or from peripheral blood mononuclear cells (PBMC) from a peripheral blood sample; and enriching the one or more types of rare circulating cell in the PBMC and/or in the peripheral blood sample, thereby providing an enriched population of rare circulating cells from the peripheral blood and/or PBMC.

Embodiments of the invention also relate to enriched populations of rare circulating cells from peripheral blood or a peripheral blood mononuclear cells (PBMC) cell fraction, prepared as described herein.

Embodiments of the invention also relate to various uses of enriched populations of rare circulating cells from peripheral blood or a peripheral blood mononuclear cells (PBMC) cell fraction as described herein, wherein the enriched populations of rare circulating cells are prepared as described herein.

In some embodiments, rare circulating cell enrichment includes either antibody-conjugated bead-based enrichment or FACS sorting, or sequential antibody-conjugated bead-based enrichment and FACS sorting. In some embodiments, rare circulating cell enrichment includes FACS-sorting rare circulating cells into one or more tubes prior to cell isolation. In some embodiments, rare circulating cell enrichment includes pooling multiple samples into a single assay tube and demultiplexing after analysis (in silico) based on oligo-conjugated antibody-based demultiplexing or genotype (SNP) based demultiplexing using genetic variance between individuals.

In some embodiments, the enriched population of rare circulating cells is introduced or re-introduced into a sample comprising peripheral blood and/or PBMC.

In some embodiments, the peripheral blood and/or PBMC includes one or more peripheral hematopoietic stem and progenitor cell (pHSPC), CD14+ monocyte (CD14 M.), CD16+ monocyte (CD16 M.), CD34+ HSPC, CD34− HSPC, B cell (B), CD4+ T cell (CD4), CD8+ T cell (CD8), dendritic cell (DC), natural killer cell (NK), plasma B cell (PC), plasmacytoid dendritic cells (pDC), hematopoietic stem cells/multipotent progenitor cell (HSC/MPP), lymphoid-primed multipotent progenitor cell (LMPP), megakaryocyte-erythroid progenitor cell (MEP), erythroid progenitor cell (Ery), granulocyte-monocyte progenitor cell (GMP), basophil-eosinophil-mast cell progenitor cell (BEM), or common myeloid progenitor (CMP). In some embodiments, the rare circulating cell includes one or more peripheral hematopoietic stem and progenitor cell (pHSPC), CD14+ monocyte (CD14 M.), CD16+ monocyte (CD16 M.), CD34+ HSPC, CD34− HSPC, B cell (B), CD4+ T cell (CD4), CD8+ T cell (CD8), dendritic cell (DC), natural killer cell (NK), plasma B cell (PC), plasmacytoid dendritic cells (pDC), hematopoietic stem cells/multipotent progenitor cell (HSC/MPP), lymphoid-primed multipotent progenitor cell (LMPP), megakaryocyte-erythroid progenitor cell (MEP), erythroid progenitor cell (Ery), granulocyte-monocyte progenitor cell (GMP), basophil-eosinophil-mast cell progenitor cell (BEM), or common myeloid progenitor (CMP). In some embodiments, the rare circulating cell is a pHSPC or pDC. In some embodiments, the pHSPC is a CD34+ or CD34− pHSPC.

In some embodiments, the peripheral blood sample can be obtained directly from a subject or can be from cryopreserved PBMC and/or cryopreserved peripheral blood.

In some embodiments, acquiring the single cell and/or bulk transcriptomic, genetic, and/or protein expression, and/or metabolic, epigenomic, and/or other functional assay data includes one or more bulk and/or single cell assay. In some embodiments, the bulk and/or single cell assay includes bulk and/or single cell RNA and/or ATACseq analysis. In some embodiments, acquiring the single cell and/or bulk transcriptomic, genetic, and/or protein expression, and/or metabolic, epigenomic, and/or other functional assay data includes one or more single cell assay and can be combined with one or more single cell-based workflows.

Some embodiments of the methods further include parallel sample preparation and scale up enabled by pooling of multiple samples and demultiplexing after analysis (in silico) based on oligo-conjugated antibody-based demultiplexing or genotype (SNP) based demultiplexing using genetic variance between individuals. Some embodiments of the methods further include subject genome sequencing to generate a reference genotype for genotype-based demultiplexing of single cell datasets from pooled samples. In some embodiments, genome sequencing includes whole genome sequencing, exome sequencing, bulk ATACseq, and/or SNP microarray.

In some embodiments, analyzing the enriched rare circulating cells includes analyzing expression of one or more of protein, mRNA, DNA (sequence or post-translational modifications), chromatin (e.g. histone modifications, accessibility, 3D structure/looping, etc.), metabolites, and/or lipids. In some embodiments, analyzing the enriched rare circulating cells includes analyzing chromatin, DNA, mRNA expression, and/or ATAC-seq data. In some embodiments, analyzing the enriched rare circulating cell mRNA and assay for transposase-accessible chromatin sequencing (ATAC-seq) data includes combined single cell mRNA/ATAC-seq data processing; UMAP visualization; single cell and/or bulk ATAC-seq; demultiplexing; and/or identifying differentially accessible regions, differentially expressed genes, and/or ATAC peak-gene/transcript associations. In some embodiments, transcriptional, genetic, protein, and/or epigenomic signatures can be determined by gene ontology (GO) analysis. In some embodiments, analyzing the enriched rare circulating cells includes combined single nuclei (sn) RNA and assay for transposase-accessible chromatin sequencing (ATAC-seq) (chromium single cell multiome ATAC+ gene expression) for PBMC, sorted PBMC subset “bulk” ATAC-seq, multiplexed immunoassay-based quantitation of plasma proteins, and/or immunophenotyping by flow cytometry.

In some embodiments, the enriched rare circulating cells have differential enrichment of epigenetic and transcriptional signatures associated with antigen presentation, activation, differentiation, and/or anti-viral responses. In some embodiments, the cellular molecular features and/or functional characteristics of the enriched rare circulating cells include increased granulo- and myelopoiesis in pHSPC, and/or monocyte phenotypes of inflammation, migration, and differentiation, and/or altered proportions or phenotypes of pHSPC subsets related to changes in hematopoiesis. In some embodiments, the increased pHSPC subsets related to changes in hematopoiesis include HSC, MPP, GMP, CMP, BEM, MEP, Ery, and/or LMPP.

In some embodiments, the cellular molecular features and/or functional characteristics of the enriched rare circulating cells and/or pHSPC-enriched PBMC can be used in a diagnostic assay. In some embodiments, the method can be used to characterize a single rare circulating cell.

Some embodiments of the methods also include using the enriched rare circulating cells in one or more functional assay. In some embodiments, the functional assay includes a differentiation potential (e.g. colony forming assay (CFA)), stimulation responsiveness (e.g. cytokine secretion/production), metabolism (e.g. oxygen consumption), migration/motility, histone modification, and/or DNA methylation assay.

In some embodiments, the cellular molecular features and/or functional characteristics of the enriched rare circulating cells can be compared to the cellular molecular features and/or functional characteristics from one or more stem cells from a bone marrow sample. In some embodiments, the rare circulating cells are isolated from a subject, and the bone marrow stem cells are from the same subject. In some embodiments, the rare circulating cells and the bone marrow stem cells can be from different clinical groups.

In some embodiments, the characterization of cellular molecular features and/or functional characteristics for the enriched rare circulating cells can be used in place of or in addition to characterization of stem cells obtained from bone marrow. In some embodiments, the characterization of cellular molecular features and/or functional characteristics for the enriched rare circulating cells can be used in place of characterization of stem cells obtained from bone marrow aspiration and/or biopsy. In some embodiments, the characterization of cellular molecular features and/or functional characteristics for the enriched rare circulating cells can be used in place of or in combination with a diagnostic assay based on characterization of one or more stem cells obtained from bone marrow aspiration and/or biopsy. In some embodiments, the characterization of cellular molecular features and/or functional characteristics for the enriched rare circulating cells is used in place of a diagnostic assay based on characterization of stem cells obtained from bone marrow aspiration and/or biopsy in diagnosis and/or treatment of inflammatory diseases, types of cancer (e.g. lymphoma, leukemia, myeloma, metastatic cancer), metabolic diseases (e.g. anemia, hemochromatosis), and/or blood disorders/conditions (leukopenia, leukocytosis, thrombocytopenia, thrombocytosis, pancytopenia, polycythemia).

In some embodiments, the sample can be from a subject post-COVID-19 infection and having one or more symptoms of long covid, and the rare circulating cells can include one or more pHSPC. In some embodiments, the characterization of cellular molecular features and/or functional characteristics for the enriched rare circulating cells can be used for single-cell profiling of human pHSPC from peripheral blood post-COVID-19 infection. In some embodiments, the sample can be from a subject post-COVID-19 infection and having one or more symptoms of long covid, and the characterization of cellular molecular features and/or functional characteristics for the pHSPC includes analysis of pHSPC transcriptomic, epigenomic, and/or protein data.

In some embodiments, the pHSPC transcriptomic, epigenomic, and/or protein data can be from 1 week, 2 weeks, 3 weeks, 1 month, 2 months, 3 months, 6 months, 9 months, 12 months, or longer after COVID-19 infection. In some embodiments, the pHSPC transcriptomic, epigenomic, and/or protein data can be from 2-4 months post hospital admission, or from 5-12 months after COVID-19 infection.

In some embodiments, the characterization of cellular molecular features and/or functional characteristics for the pHSPC post-COVID-19 infection includes pHSPC transcriptional and epigenetic signatures. In some embodiments, pHSPC transcriptional and epigenetic signatures post-COVID-19 infection include enrichment for one or more inflammatory genes or genetic regulatory elements; one or more genes or genetic regulatory elements related to antigen presentation, activation, differentiation, and/or anti-viral responses; and/or gene enrichment and/or enrichment of accessibility at promoters, enhancers, and/or differently accessible regions (DAR). In some embodiments, the pHSPC transcriptional and epigenetic signatures post-COVID-19 infection include enrichment for one or more inflammatory genes; IL-6R signaling genes; genes related to antigen presentation, activation, differentiation, and/or anti-viral responses; genes related to differentiation, migration, activation, and cytokine-mediated signaling; genes related to myeloid differentiation, activation, and cytokine production; genes related to programs of myeloid dendritic cell activation; genes related to platelet activation; and/or genes related to neutrophil/GMP/activation; chromatin accessibility genes encoding cytokines, adhesion molecules, and/or differentiation factors; and/or wherein the PBMC transcriptional and epigenetic signatures post-COVID-19 infection comprise dysregulation of hematopoiesis and genes linked to granulopoiesis and myelopoiesis; reduced expression of negative feedback factors; increased chromatin accessibility at chemokines; chemokine receptor genes; interferon stimulated genes; and/or immunomodulatory genes.

In some embodiments, one or more inflammatory genes can include S100A12, CTSC, IL6, CD28, NLRP12, IRF1, STAT1, NFKB1, NFKBIA, PPARG, IL1RAP, and/or MAPKAPK2; genes related to IL-6R signaling can include CEBPb, STAT3, and/or CRP; late enriched monocyte genes can include M.SC3; genes related to antigen presentation, activation, differentiation, and/or anti-viral responses can include CD74, LGMN, B2M, IFI30, HLA, LYZ, CD14, S100As, and/or IL1B; the genes related to differentiation, migration, activation, and cytokine-mediated signaling can include PTPRC, ITGAM, CCL26, IL1RL2, and/or IFI16; the genes related to myeloid differentiation, activation, and cytokine production can include IKZF1, IL4R, RARA, STAT3, and/or KLF13; the genes related to programs of myeloid dendritic cell activation can include RELB, CD2, CAMK4, and/or SLAMF1; the genes related to platelet activation can include GP1BB, PDGFB, CD40, and/or MYH9; the genes related to neutrophil/GMP/activation can include S100A9, S100A8, CAMK1D, and/or CD74; the chromatin accessibility genes encoding cytokines can include CCL3, IL10, and/or IFNG; the chromatin accessibility genes encoding adhesion molecules can include CD1D, DOCK5, ADAM9, and/or ITGAL; and the chromatin accessibility genes encoding differentiation factors can include KLF13. CREB1, PRKCA, and/or FOXP1; the genes linked to granulopoiesis and myelopoiesis can include CD14, KLF2, CEBPD, and CCL5; the negative feedback factors comprise DUSP1 and/or NFKBIA; the chemokines can include CCL4 and/or CXCL8; the chemokine receptors can include CCR4, CCR6, and/or CXCR3; the interferon stimulated genes can include NLRC5, SOCS1, and/or IFITM1; and/or the immunomodulatory genes can include METRNL, NLRC3, KLF4, and/or TNFAIP3. In some embodiments, the characterization of cellular molecular features and/or functional characteristics for the pHSPC post-COVID-19 infection can include enrichment for chromatin binding or inferred chromatin binding (based on motif enrichment in DAR and/or footprints) of NRF1, STAT3, NFkB, CEBPb, AP-1, IRF1, IRF2, IRF3, IRF4, IRF5, IRF6, IRF7, IRF8, and/or CTCF.

In some embodiments, pHSPC transcriptional and epigenetic signatures post-COVID-19 infection include increased pHSPC subsets related to changes in hematopoiesis. In some embodiments, the increased HSPC subsets related to changes in hematopoiesis include HSC, MPP, GMP, CMP, BEM, MEP, Ery, and/or LMPP. In some embodiments, the characterization of cellular molecular features and/or functional characteristics for the pHSPC post-COVID-19 infection includes increased granulo- and myelopoiesis in pHSPC, and monocyte phenotypes of inflammation, migration, and differentiation.

Some embodiments of the method further include treating the subject having long covid symptoms by: identifying one or more therapeutic targets based on the pHSPC transcriptional and epigenetic signatures; and treating the subject by administering a therapeutically effective amount of a treatment directed to the one or more targets identified from the pHSPC transcriptional and epigenetic signatures. In some embodiments, the therapeutic target can include IL-6, IL-6R, IL-1, IL-12/23, IL-17, IL-23, IL-4/13, TNF, JAK, and/or another cytokine. In some embodiments, the therapeutic target includes IL-6, and the treatment includes an IL-6R blocking antibody, and/or G-CSF, GM-CSF, and/or chemokine/cytokine targeting. In some embodiments, the IL-6R blocking antibody includes Tocilizumab and/or Sarilumab. In some embodiments, the treatment comprises an anti-inflammatory biologic and/or a steroid.

In some embodiments, the characterization of cellular molecular features and/or functional characteristics for the enriched rare circulating cells can be associated with clinical data and laboratory results to identify one or more mechanisms of disease, biomarkers, and/or therapeutic targets related to changes in hematopoiesis. In some embodiments, a subject can be subsequently treated by administering a therapeutically effective amount of a treatment directed to the one or more targets identified from the association of the cellular molecular features and/or functional characteristics for the enriched rare circulating cells with clinical data and laboratory results.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. An example computer system, upon which embodiments, or portions of the embodiments, may be implemented, in accordance with various embodiments.

FIG. 2. Cohort overview and plasma and flow data. FIG. 2A) Summary table of samples used for different analyses. FIG. 2B) Swimmer plot showing hospitalization periods, and sample collection time point for each patient in nonCOV, Early, and Late. Early-ICU group is colored with dark red, indicating period of ICU admission. FIG. 2C) Viral S protein-specific antibody assays showing antibody level, neutralizing activity, and binding quality. (ANOVA, p*<0.05) FIG. 2D-E) A heatmap (FIG. 2D) showing plasma cytokine levels measured by Luminex platform. Cytokine level per subject grouped by group is shown as boxplots. (*p<0.05, Wilcoxon's test, Healthy group was used as a reference) For heatmap, median cytokine level per group was normalized by Z-score. Cytokines with any significant difference between healthy and any clinical groups are indicated by asterisk. FIG. 2F) Flow cytometry results showing frequencies of CD14⁺ monocytes, CD34⁺ HSPC, and monocyte subsets across groups. (ANOVA, *p<0.05)

FIG. 3. Durable epigenetic alterations in monocytes following severe COVID-19. FIG. 3A) Study overview depicting the study cohort and ATAC-seq workflow for profiling the epigenomes of CD14⁺ monocytes. Study participants included healthy individuals (“Healthy”) and those recovering from severe COVID-19 early in convalescence (“Early”, 2-4 months post hospital admission), in late convalescence (“Late”, 5-12 months post admission), and those recovering from non-COVID-19 critical illness (“nonCoV”, 2-4 months post admission). “n=” for combined bulk and pseudobulk (from single nucleus) ATAC-seq datasets in each clinical group and for single cell analyses are shown. FIG. 3B) Principal component analysis (PCA) representation of bulk ATAC-seq data (circles) and pseudo-bulk data from snATAC-seq dataset (triangles) for CD14⁺ monocytes based on a combined set of differential peaks (n=2029) (left). Boxplots (right) show PC1 and PC2 values for each individual in the clinical groups. (Wilcoxon's test, *p<0.05; Healthy group as a reference). FIG. 3C) A heatmap displaying the unsupervised hierarchical clustering of the differentially accessible regions (DAR) in CD14⁺ monocytes by clinical group. Each row represents normalized peak accessibility for each individual (columns) with DAR clustered according to chromatin accessibility patterns (trends across clinical groups) into four clusters (C1-C4). Each individual was annotated for age, clinical group, and assay type. (For DAR, FDR<0.1 was used.) FIG. 3D) Gene ontology (GO) analysis of genes associated to cluster-specific DARs in CD14⁺ monocytes, C1-C4, from (C). Only significant (p<0.05) enrichments are visualized. C2 and C3 were annotated as “persistent” clusters based on sustained increased accessibility relative to Healthy in both Early and Late. C4 was annotated as “transient” since chromatin changes in Late resemble healthy samples. FIG. 3E) Top: Violin plots for normalized read density of all DARs in clusters C1-C4 split by clinical group. Each DAR/peak (group average) is represented by a linked line across each series (top). Bottom: Boxplots represent individuals within groups for cluster (C1-C4) average based on normalized DAR density score. Example genes associated to DARs in C2 and C4 are shown between violin plots and box plots. (Wilcoxon's test, *p<0.05; Healthy group as a reference.) FIG. 3F) Examples of ATAC-seq genome browser tracks of DARs in C2 and C4 in CD14+ monocytes (Healthy, black line; Early, red; Late, yellow; nonCoV, dark blue; black line representing Healthy is shown for each track for reference). Boxplots display peak/DAR normalized densities for each individual. (Wilcoxon's test, *p<0.05; Healthy group as a reference).

FIG. 4. Durable changes in chromatin accessibility and expression in CD14⁺ monocytes following severe COVID-19. FIG. 4A) Gene set enrichment analysis (GSEA) of PC1- and PC2-associated differentially accessible regions (DAR) from PCA analysis in CD14⁺ monocytes. setSize indicate number of DAR-associated genes in each GO term. NES stands for normalized enrichment score, and positive NES shows enrichment of DAR-associated genes with positive PC values (same applies to negative NES and PC value). FIG. 4B) Volcano plots showing differentially expressed genes (DEG) in CD14⁺ monocytes between Healthy and Early/Late/nonCov groups. Significantly differential genes are highlighted with different colors specific to groups and labels. (Wilcoxon's test, *adjusted p<0.05) FIG. 4C) Volcano plots displaying DORC activity in CD14+ monocyte between the Healthy and Early/Late groups. DORC-associated genes with significant changes are highlighted and labeled with text. (Wilcoxon's test, *adjusted p<0.05) FIG. 4D) Gene ontology analysis of gene expression (blue dots), and chromatin accessibility (DORC) (grey dots) specific to each CD14⁺ Monocyte subcluster. FIG. 4E) Genome tracks of IL1B region across groups (Healthy, outline; Early, red; Late, yellow; nonCoV, dark blue) in HSPC (top) and CD14⁺ monocytes (bottom). Differentially accessible peak across groups in both cell types are highlighted with box.

FIG. 5. Basis of combined single-nuclei ATAC/RNA-seq, generation of domains of regulatory chromatin (DORC) data, and HSPC subcluster annotation. FIG. 5A) UMAP plots displaying unbiased clustering (top) and group distribution (bottom) using snRNA-seq (left) and snATAC-seq (right). FIG. 5B) Count matrix showing number of differentially expressed genes in various comparisons across cell types. B cell (B), CD4 T cell (CD4), CD8 T cell (CD8), dendritic cell (DC), natural killer cell (NK), plasma cell (PC), plasmacytoid DC (pDC). FIG. 5C) Stacked bar plot showing cell type composition in each individual patient sample grouped by group. FIG. 5D) Boxplots showing percent of major cell type within total PBMC per subject by group. (Wilcoxon's test, *p<0.05; Healthy group as a reference). FIG. 5E) Schematic describing workflow for analyzing distal regulatory elements and gene expression using snRNA/ATAC-seq data including domains of regulatory chromatin (DORC). ATAC-seq peaks were linked to putative target genes in cis, based on the co-variation of chromatin accessibility and gene expression levels across individual cells developmentally related HSPC and monocytes were then co-clustered based on DORC scores. FIG. 5F) Genome track showing chromatin accessibility of CEBPA-associated DORC in HSC/MPP and CD14⁺ monocytes. Curved lines (loops) above indicate peaks with significant correlation (p-value <0.05). The grey bar (below gene track) represents peaks. FIG. 5G) The number of significant peak-gene connections for all genes. FIG. 5H) The number of significantly correlated peaks (p-value <0.05) for each gene. FIG. 5I) Heatmap showing expression of marker genes for each HSPC subcluster. FIG. 5J) Unbiased clustering of HSPC UMAP of snRNA-seq data. FIG. 5K) HSPC UMAP with annotation transferred from bone marrow single-cell RNA-seq data (Granja et al., 2019). FIG. 5L) HSPC UMAP with subcluster annotation guided by ATAC-seq data of FACS-sorted bone marrow HSPC subpopulations (Buenrostro et al., 2018). FIG. 5M) HSPC UMAP showing density of CD164-expressing cells.

FIG. 6. Altered CD14⁺ monocyte programs and function following severe COVID-19. FIG. 6A) Single cell RNA (snRNA-seq) UMAP visualization of myeloid clusters including CD14⁺ monocytes (CD14⁺ Mono.), CD16⁺ monocytes (CD16⁺ Mono.), and dendritic cells (DC) from all clinical groups. FIG. 6B) Boxplots showing percent of each myeloid subcluster within the myeloid population, for each individual in each group. (Wilcoxon's test, *p<0.05; Healthy group as a reference) FIG. 6C) Gene ontology enrichment analysis of genes with upregulated transcription (blue dots), and increased chromatin accessibility (grey dots) in CD14⁺ monocytes of each group compared to Healthy. Upregulated transcription from snRNA-seq; increased chromatin accessibility from domains of regulatory chromatin (DORC) scores derived from snATAC-seq. FIG. 6D) Inflammation score for each clinical group obtained from expression profiles of inflammatory gene sets. (Wilcoxon's test, *p<0.05; Healthy group as a reference). FIG. 6E) Heatmap displaying the average expression (normalized score) of top clinical group-defining differentially expressed genes (DEG) ranked by adjusted p-value in CD14⁺ monocytes. FIG. 6F) Three monocyte subclusters (M.SC1-3) were defined within CD14⁺ monocytes and annotated in the myeloid cell UMAP. M.SC3, also termed “Late enriched” due to enrichment in the cells from the Late group. FIG. 6G) Expression of myeloid subcluster-defining marker genes for CD14⁺ monocyte subclusters M.SC1-3, CD16⁺ monocytes, and DC. The dashed box highlights similarities between “Late-enriched M.SC3” and DC subclusters. FIG. 6H) Boxplots showing percent of CD14⁺ monocyte subclusters, M.SC1-3, among total myeloid cells per subject, by clinical group. (Wilcoxon's test, *p<0.05; Healthy group as a reference.). FIG. 6I) A scheme for ex-vivo stimulation of CD14⁺ monocytes isolated from PBMC with R848 and IFNa to model an anti-viral response (left). RT-qPCR analysis of IL1B expression in CD14⁺ monocytes of each clinical group after 6 hours of stimulation. (t-test, *p<0.05; Healthy group as a reference.)

FIG. 7. Epigenomic and transcriptomic analysis of rare circulating hematopoietic stem and progenitor cells (HSPC) establishes their similarity to bone marrow HSPC. FIG. 7A) Schema depicting subject-paired bone marrow aspirate and peripheral blood analysis with enrichment of CD34⁺ HSPC from bone marrow mononuclear cells (BMMC) and peripheral blood mononuclear cells (PBMC) followed by combined single-nuclei RNA/ATAC-seq (Multiome). Approximate percentage of HSPCs from the original and enriched samples are indicated. PBMC analysis with Progenitor Input Enrichment (PBMC-PIE), BMMC analysis with Progenitor Input Enrichment (BMMC-PIE). FIG. 7B) UMAP plots for combined snRNA/ATAC-seq data of paired BMMC-PIE and PBMC-PIE for all cells, both enriched for HSPCs from each tissue (n=2 individuals). Plots were annotated for major cell types with HSPC outlined (upper left), and tissue origin (BMMC and PBMC) revealing co-clustering of BMMC and PBMC HSPC (upper right). HSPC only cluster UMAP plots from snRNA/ATAC-seq data were annotated for HSPC subclusters (bottom). B cells (B), CD14+ monocytes (CD14 M.), CD16+ monocytes (CD16 M.), CD4+ T cells (CD4), CD8+ T cells (CD8), dendritic cell (DC), hematopoietic stem and progenitor cells (HSPC), natural killer cells (NK), plasma B cells (PC), plasmacytoid dendritic cells (pDC), erythroid progenitor cells (Ery), basophil-eosinophil-mast cell progenitor cells (BEM), lymphoid-primed multipotent progenitor cells (LMPP), megakaryocyte-erythroid progenitor cells (MEP), hematopoietic stem cells/multipotent progenitor cells (HSC/MPP), granulocyte-macrophage progenitor cells (GMP). FIG. 7C) Expression of HSPC subcluster marker genes in each HSPC subcluster from peripheral blood, PBMC HSPC (top) and bone marrow, BMMC HSPC (bottom). FIG. 7D) Summary of study participants profiled using PBMC-PIE workflow from each clinical group: Early, Late, nonCoV, and Healthy groups. PBMC-PIE samples are profiled using bulk or snATAC-seq (pseudobulk) using Multiome. FIG. 7E) UMAP of the PBMC-PIE snRNA-seq samples from 24 individuals representing 197,360 cells. Enriched HSPCs representing 28,069 cells (pink cluster) are highlighted with dashed line. FIG. 7F-G) snRNA/ATAC-seq UMAP plots of cells in the HSPC cluster. These cells are annotated based on two independent and concordant studies: marker gene annotation from our snRNA-seq dataset (FIG. 7F), and based on chromatin accessibility referenced from bone marrow HSPC subtype ATAC-seq data (Buenrostro et al., 2018) (FIG. 7G).

FIG. 8. Sustained epigenetic alterations in CD34⁺ HSPC following severe COVID-19. FIG. 8A) Study overview depicting the clinical groups and ATAC-seq workflow used to profile the epigenomes of CD34⁺ HSPC. Study participants included healthy individuals (“Healthy”) and those recovering from severe COVID-19 early in convalescence (“Early”, 2-4 months post hospital admission), in late convalescence (“Late”, 5-12 months post admission), and those convalescing from non-COVID-19 critical illness (“nonCoV”, 2-4 months post admission). “n=” represents combined bulk and pseudobulk (from single cell) ATAC-seq datasets in each clinical group. FIG. 8B) Principal component analysis (PCA) of all CD34+ ATAC-seq samples (bulk: circles, pseudo-bulk: triangles) using the combined set of differential peaks (n=1319 peaks) (left). Boxplots (right) show PC1 and PC2 values for each individual in each clinical group. (Wilcoxon's test, *p<0.05; Healthy group as a reference) FIG. 8C) Top: Violin plots for normalized read density of all DARs for three subclusters of HSPCs (C1-C3) by clinical group. Each DAR/peak (group average) is represented by a linked line across each series. Bottom: Boxplots represent individuals within all clinical groups for normalized peak/DAR average accessibility in each cluster (C1-C3). Example genes from C1 and C2 are shown between violin plots and box plots. (Wilcoxon's test, *p<0.05; Healthy group as a reference.) FIG. 8D) Gene ontology (GO) analysis of genes associated to cluster-specific DAR in HSPC, C1-C3, from FIG. 8C. Only significant (p<0.05) enrichments are visualized. C1 was annotated as “persistent” based on sustained increased accessibility relative to Healthy in both Early and Late, and C2 was annotated as “transient” since chromatin accessibility data in Late resemble that of healthy samples. FIG. 8E) ATAC-seq genome browser tracks for example cluster-specific DAR in HSPC (Healthy, black line; Early, red; Late, yellow; nonCoV, dark blue; black line representing Healthy is shown for each track for reference). Boxplots display peak/DAR normalized densities for each individual. (Wilcoxon's test, *p<0.05; Healthy group as a reference).

FIG. 9. Durably altered phenotypes and programs in hematopoietic stem and progenitor cells following severe COVID-19. FIG. 9A) UMAP visualization of HSPC snRNA-seq data with subcluster annotations. Erythroid progenitor cells (Ery), basophil-eosinophil-mast cell progenitor cells (BEM), lymphoid-primed multipotent progenitor cells (LMPP), megakaryocyte-erythroid progenitor cells (MEP), hematopoietic stem cells/multipotent progenitor cells (HSC/MPP), granulocyte-macrophage progenitor cells (GMP). FIG. 9B) Gene ontology enrichment analysis of genes with upregulated transcription (blue dots), and increased chromatin accessibility (grey dots) in HSPC of each clinical group compared to Healthy. Upregulated transcription from snRNA-seq; increased chromatin accessibility from domains of regulatory chromatin (DORC) scores derived from snATAC-seq. FIG. 9C) Boxplots showing percent of each HSPC subcluster, of total HSPC, for each individual in all clinical groups. (Wilcoxon's test, *p<0.05; Healthy group as a reference.) FIG. 9D-E) UMAP plots (left) displaying GMP expression module (FIG. 9D) and the DORC scores for the neutrophil module (FIG. 9E) for each cell. Violin plots (right) showing the distribution of module scores per cell in each clinical group. GMP and neutrophil modules were defined by GMP cluster markers, and DORC-associated genes included in neutrophil-related GO terms, respectively. HSC/MPP and GMP are highlighted with a dashed line, given their enrichment in post-COVID-19 groups. FIG. 9F) Heatmap displaying the chromVAR score (Z-score-normalized median) for selected TFs characteristic of each HSPC subcluster. FIG. 9G-H) chromVAR scores for FOS::JUN (MA0099.3) and CEBPA (MA0102.4) in HSPC. Scores are projected onto HSPC snRNA-seq UMAP plots (left). Average score per subject (middle) and score distribution across individual cells (right) by group are shown as box plot and violin plot, respectively (Wilcoxon's test, *p<0.05; Healthy group as a reference). HSC/MPP and GMP are highlighted with a dashed line, given their enrichment in post-COVID-19 groups.

FIG. 10. Persistent changes in chromatin accessibility and expression in HSPC following severe COVID-19 and reflecting altered hematopoiesis. FIG. 10A) Gene set enrichment analysis (GSEA) of PC1- and PC2-associated differentially accessible regions (DAR) from PCA analysis in HSPC. setSize indicate number of DAR-associated genes in each GO term. NES stands for normalized enrichment score, and positive NES shows enrichment of DAR-associated genes with positive PC values (same applies to negative NES and PC value). FIG. 10B) Volcano plots showing differentially expressed genes in HSPC between Healthy and Early/Late/nonCov groups. Significantly differential genes are highlighted with different colors specific to groups and top differential genes are labeled with gene names. (FDR<0.05) FIG. 10C) Heatmap showing differentially expressed genes associated with hematopoietic regulation in different groups. Genes that are discussed in the main text are bolded. (FDR<0.05) FIG. 10D) Box plots showing average expression of select genes per subject. Genes related to myeloid differentiation are selected from differentially expressed genes in Late. (Wilcoxon's test, *p<0.05, Healthy group as a reference) FIG. 10E) Projections of group density on the snRNA-seq HSPC UMAP plot. A scaled color indicates the density of cells that have cells from the same group within 50 adjacent cells.

FIG. 11. Transcription factor programs are durably altered following severe COVID-19 and are shared between HSPC and CD14+ monocytes. FIG. 11A) Volcano plots showing differentially active transcription factors (TFs) in CD14⁺ monocytes and HSPC between Healthy and Early/Late groups (based on chromVAR score log 2FC). Significantly differential TFs are highlighted with different colors specific to groups. HSPC (Early and Late comparisons) are shown at left; CD14⁺ monocytes (Early and Late comparisons) are shown at right. (Wilcoxon's test, *adjusted p<0.05, Healthy as a reference.) FIG. 11B-C) chromVAR scores for IRF2 (MA0051.1) in HSPC (FIG. 11B) and CD14⁺ monocytes (FIG. 11C). Scores are projected onto UMAP plots of each cluster (left). Distribution of chromVAR scores across all cells by group are shown as violin plots (right) (Wilcoxon's test, *p<0.05; Healthy group as a reference). HSC/MPP and GMP are highlighted with a dashed line, given their enrichment in post-COVID-19 groups. FIG. 11D) Boxplots showing average expression of representative marker genes of monocyte subcluster 3 (M.SC3) per individual. M.SC3 contained increased frequencies of monocytes from the Late group (FIG. 6F-G). (Wilcoxon's test, *p<0.05; Healthy group as a reference.) FIG. 11E-F) M.SC3 module score in Myeloid cluster (FIG. 11E) and HSPC (FIG. 11F). M.SC3 module score is projected onto UMAP plots (left), showing module activity in M.SC3 monocytes and HSC/MPP and GMP subclusters. Violin plots show M.SC3 module score distribution for individual CD14+ monocyte cells (FIG. 11E) and HSPC (FIG. 11F) by group. (Wilcoxon's test, *p<0.05; Healthy group as a reference.)

FIG. 12. Differential transcription factor activity post-COVID-19 shared in HSPC and progeny CD14⁺ monocytes. FIG. 12A) Volcano plots showing differentially active TFs in HSPC and CD14⁺ monocytes between Healthy and nonCov. Significantly differential TFs are highlighted and labeled with TF names. (Wilcoxon's test, *adjusted p<0.05, Healthy group as a reference) FIG. 12B) Boxplots for mean chromVAR score of select TFs in HSPC and CD14⁺ monocytes for individual subject grouped by groups (Wilcoxon's test, *p<0.05, Healthy group as a reference). FIG. 12C) Transcription factor footprints (HINT) and violin plots for TF motif-associated chromatin accessibility (chromVAR) activity in HSPC and CD14⁺ monocytes across groups. (Wilcoxon's test, *p<0.05, Healthy group as a reference).

FIG. 13. Differential transcription factor activity post-COVID-19 in various PBMC cell types. Heatmap showing motif activities of TF families across each clinical group in individual cell types. ChromVAR score was z-score-normalized by row after taking the median for each group.

FIG. 14. Colony forming assays with purified peripheral blood HSPC. FIG. 14A) Healthy and post-Covid-19 GM colonies. FIG. 14B) erythroid and granulocyte, macrophage colonies in healthy and post-Covid-19 samples. FIG. 14C) Imaging of erythroid and granulocyte, macrophage colonies.

FIG. 15. raw data ATACseq read mapping at the TL1A locus indicating wild-type allele (gray, A) and variant allele (dark line, G).

FIG. 16. Table of SNP variant calls for 8 individuals across 5 SNPs at the TL1A locus. Read density is too sparse to call the non-regulatory SNPs (lower 4 rows), but read depth is sufficient at the regulatory enhancer (row 1) to call these SNPs and genotype information for each individual.

FIG. 17. Multiple hypothesis testing: Several examples of clinical and treatment variables and multiple hypothesis testing p-val (shown within figure) for association with GMP frequency. Adj p-value (for single variable comparing +/−condition) plotted on the x-axis. This reveals that the variance in GMP frequencies in post-COVID-19 individuals is best explained by IL-6R blockade therapy status (“anti-IL6”).

FIG. 18. Left, granulocyte-monocyte progenitor (GMP) frequencies (among total HSPC/CD34+ cells) in different clinical groups. From left to right: This shows that (i) healthy donors have slightly elevated levels of GMP in peripheral blood compared to bone marrow; and (ii) comparing peripheral HSPC across clinical disease groups that these all have increased frequencies of GMP and (iii) that IL6R blockade (right-most box plot) reduces GMP frequencies significantly. Right, CEBPA transcription factor binding (inferred from ATACseq data, chromvar score) in late convalescent (4-12 months post-acute) individuals also decreases with IL-6R blockade treatment.

FIG. 19. CEBP transcription factors which regulate monocyte and neutrophil differentiation change in response to post-infection status and treatment with IL-6R blockade. Importantly, CEBPB (also known as “nuclear factor interleukin 6”, or “NF-IL6”) is increased in post-infection states, and then decreased by IL-6R blockade, indicating a signature of response to therapy

FIG. 20. Murine Hepatitis Virus-1 as a model of post-coronavirus changes in hematopoiesis and the immune system.

FIG. 21. Single cell (combined RNA/ATACseq) analysis of bone marrow from AJ mice 30 days after MHV1 infection. This is analogous to PBMC-PIE based analysis of the human cohort.

FIG. 22. Frequencies of GMP (“monocyte lineage” and “neutrophil lineage” progenitors) increases in mice recovered from MHV1 infection (30 days post infection) compared to naïve, uninfected mice, and is decreased by IL-6R blockade.

FIG. 23. Immune cell infiltrates increase in the brain of mice recovered from MHV1 infection (30 days post infection, “AJ MHV1”) compared to naïve, uninfected mice. IL-6R blockade (“+IL6”) reduces monocyte and CD4+ T cell infiltration into the brain.

FIG. 24. Transcription factor motif accessibility (chromvar scores) or inferred chromatin binding changes in responses to post-infection status and is normalized by IL-6R therapy. Transcription factors that are mediators of inflammatory programs have increased activity following infection in both HSC (left) and neutrophil progenitors (right). This activity is altered and generally reduced by IL-6R blockade.

FIG. 25. A gene expression module defining the GMP program is increased in progenitor cells of recovered mice and reduced by IL-6R blockade.

FIG. 26. IL6R signals via the transcription factor STAT3. In post-infection mice, increased STAT3 chromatin binding (bold) is observed as expected given elevated levels of IL-6 during acute infection. Durable STAT3 chromatin binding in convalescence is reduced by earlier IL-6R treatment in acute disease. These data indicate that this experimental workflow can reveal durable post-infection and post-treatment changes in epigenetic features, including transcription factor activity.

FIG. 27. A pre-enrichment strategy to obtain the heterogeneity of pDCs in human donors at the single cell level. FIG. 27A) The flow chart of the sample preparation, library construction and data processing. Frozen PBMCs from 8 donors were sorted to obtain enriched pDCs and T cell depleted PBMCs fractions, the two fractions were then remerged at 1:2 ratio before loading to 10× single cell microfluidic chips. After library construction and sequencing, a merged dataset with cells were obtained and batch corrected using FastMNN in a base of Seurat pipeline. Doublets were removed manually and the data is visualized by UMAP plots. FIG. 27B) Cluster annotation of PBMCs. The dot plot represents expression values of selected genes (x axis) across each cluster (y axis). The color intensity indicates the scaled average expression within expressing cells. The dot size represents the percentage of cells expressing the marker genes. FIG. 27) The UMAP visualization of subclusters of donor's PBMCs; the putative identity of each cluster was assigned on the basis of markers described in FIG. 27B. FIG. 27D) UMAP plots that identify 6 subclusters of pDCs. FIG. 27E) A heat map representing scaled expression values of the top 5 genes defining each subcluster of pDCs. FIG. 27F) UMAP plot shown the ISGs expression in the total population of pDCs. FIG. 27G) The Principle component analysis of pathway activated in each subclusters.

### DETAILED DESCRIPTION

### I. Overview

Systemic inflammation can trigger innate immune memory and persistent changes in hematopoietic cells through epigenetic mechanisms. However, investigating these phenotypes in the context of human disease, including severe coronavirus disease 2019 (COVID-19), has been challenging to date.

As described herein, the present inventors have found that circulating HSPC, enriched from peripheral blood, capture the diversity of HSPC in bone marrow. This enables the investigation of hematopoiesis and HSPC epigenomic changes following COVID-19. Alterations in innate immune phenotypes and epigenetic programs of HSPC were found to persist for months to one year following severe COVID-19 and were associated with distinct transcription factor activities (including IRF, AP-1, and CTCF), altered regulation of inflammatory programs, and durable increases in myelopoiesis and granulopoiesis. Peripheral blood HSPC (also referred to herein interchangeably as “peripheral HSPC”, “pHSPC”, “circulating HSPC”, and “HSPC”) retained epigenomic alterations that were conveyed, through differentiation, to progeny innate immune cells. Epigenetic reprogramming of pHSPC can underly altered immune function following infection and be broadly relevant, especially for millions of COVID-19 survivors with incomplete recovery.

Recent studies have established that innate immune cells and their progenitors can maintain durable epigenetic memory of previous infectious or inflammatory encounters, thereby altering innate immune equilibrium and responses to subsequent challenges (Bekkering et al., 2021; Netea et al., 2020). This innate immune memory, also termed trained immunity (Netea et al., 2011), has been attributed largely to persistent chromatin alterations that modify the type and scope of responsiveness of the cells that harbor them, including long-lived innate immune cells (Bekkering et al., 2021; Netea et al., 2020), epithelial stem cells (Larsen et al., 2021; Naik et al., 2017; Niec et al., 2021), and self-renewing hematopoietic progenitors and their mature progeny cells (Cirovic et al., 2020; Dos Santos et al., 2019; Kaufmann et al., 2018; Kleinnijenhuis et al., 2012, 2014; Mitroulis et al., 2018; Quintin et al., 2012). A paradox of innate immune memory has been that many of the innate immune cells that retain durable alterations are themselves short-lived (Patel et al., 2017). Addressing this, studies in mice revealed that hematopoietic stem and progenitor cells (HSPC) can be epigenetically reprogrammed upon exposure to inflammation and can lead to altered and longer-lasting phenotypes in progeny cells (Christ et al., 2018; Kaufmann et al., 2018; Mitroulis et al., 2018).

While innate immune memory phenotypes have been well-studied with in vivo mouse models, the breadth, relevance, and molecular features of such phenotypes in humans have been more elusive. Recent studies have revealed innate immune memory in humans following administration of the tuberculosis vaccine Bacillus Calmette-Guérin (BCG) (Cirovic et al., 2020; Dos Santos et al., 2019; Kong et al., 2021). However, challenges in experimental access to human HSPC have limited our understanding of dynamic and cumulative hematopoietic progenitor cell phenotypes, especially in the context of infectious disease.

As described in Examples 1-11 herein, we identified epigenetic innate immune memory that results from SARS-CoV-2 infection by characterizing the cellular and molecular features of the post-infection period of COVID-19. We focused on the analyses of chromatin and transcription at the single-cell level in monocytes and their pHSPC progenitors. We generated high-resolution transcriptomic and chromatin accessibility maps of diverse pHSPC subsets and peripheral blood mononuclear cells (PBMCs) in a cohort of severe (requiring ICU admission) convalescent COVID-19 study participants, including samples collected in early recovery (2-4mo, “Early”) or up to a year (4-12mo, “Late”) after disease onset. We compared these clinical groups to healthy participants and to participants recovering from non-COVID-19 critical illness (nonCoV, requiring ICU admission) to identify features common among patients recovering from serious disease and also unique to patients recovering from severe COVID-19.

In view of the above, this specification describes various exemplary embodiments of systems, software and methods for generating insights related to enrichment and characterization of rare circulating cells, including hematopoietic stem and progenitor cells (HSPCs) and plasmacytoid dendritic cells (pDC), from peripheral blood. This can be used, for example to assess innate immune cell epigenetic alterations in humans. In particular, embodiments of the invention encompass methods of enriching and characterizing pHSPCs post-COVID-19 infection, thereby identifying therapeutic targets for treatment of symptoms post-COVID-19 infection. The disclosure, however, is not limited to these exemplary embodiments and applications or to the manner in which the exemplary embodiments and applications operate or are described herein.

This study reveals the persistence of epigenetic and transcription programs in pHSPC and monocytes following severe disease, i.e. COVID-19, that are indicative of an altered innate immune responsiveness. Durable epigenetic and transcription programs following severe COVID-19 were linked to increased granulo- and myelopoiesis in pHSPC, and monocyte phenotypes of inflammation, migration, and differentiation. This study demonstrates that acute human viral infection can drive a durable epigenetic program in HSPC that is conveyed to progeny innate immune cells, with potential to alter subsequent immune responses. These mechanisms can influence post-infection phenotypes, ranging from protection to heterologous infections to chronic inflammation and long-term clinical sequalae.

Accordingly, embodiments of the invention relate to the use of the workflow as described herein for enriching rare cells from peripheral blood, including PBMCs, including very rare circulating HSPCs (which are about ˜0.05% of PBMC). These enriched cells can then be used for analysis and characterization of cellular molecular features and/or functional characteristics, including at single cell resolution, and for cell function studies, such as, for example, colony forming assays. This workflow is termed herein “PBMC Analysis with Progenitor Input Enrichment”, or “PBMC-PIE”. Types of cellular molecular features and/or functional characteristic data derivable from PBMC-PIE include, for example:
cell function assays, such as assays relating to differentiation potential (e.g. colony forming assays);
stimulation responsiveness assays (e.g. cytokine secretion/production);
metabolism assays (e.g. oxygen consumption);
migration/motility assays;
DNA methylation assays;
gene expression patterns (mRNA, DNA (e.g. sequence or post-translational modifications));
genome sequencing (e.g. whole genome sequencing, exome sequencing, bulk ATACseq, SNP microarray);
protein expression;
lipid assays;
chromatin histone modification assays;
chromatin accessibility (ATACseq);
chromatin 3D structure/looping assays;
inferred chromatin binding activity of specific transcription factors (derived from ATACseq data), for example, as in the post-COVID-19 determination of CEBP, AP1, and IRF transcription factor activity as described herein; and
inferred pathway activity, derived from chromatin binding and gene expression data, as described herein.

In accordance with various embodiments, non-limiting examples of systems and methods are provided for characterizing transcriptional and epigenetic signatures in one or more PBMC based on transcriptomic and epigenomic analysis. The embodiments disclosed herein reveal epigenomic alterations in innate immune and pHSPC post-COVID-19, with distinct molecular programs across disease severities. Enabled by novel approaches to study hematopoiesis from peripheral blood, one can find persisting pHSPC epigenetic programs conveyed, for months to a year, to short-lived progeny monocytes. These epigenetic changes are associated with increased myeloid cell differentiation and inflammatory and antiviral programs. As such, one can provide insights into post-infectious pHSPC and innate immune cell epigenetic alterations that are broadly relevant.

In further embodiments, the characterization of PBMC transcriptional and epigenetic signatures can be used in place of or in addition to characterization of stem cells obtained from bone marrow, such as in place of a diagnostic assay based on characterization of stem cells obtained from bone marrow aspiration and/or biopsy.

In various embodiments, a system of one or more computers can be provided that can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. In accordance with various embodiments, a method can be provided wherein the system of one or more computers is used to characterize transcriptional and epigenetic signatures in one or more PBMC based on transcriptomic and epigenomic analysis.

In various embodiments, a non-transitory computer-readable medium storing computer instructions can be provided that performs a method for characterizing transcriptional and epigenetic signatures in one or more PBMC based on transcriptomic and epigenomic analysis. The method can include receiving a set of single cell and/or bulk mRNA and ATACset data for one or more PBMC; analyzing the PBMC mRNA and ATACseq data via in depth transcriptomic and epigenomic analysis to identify differentially accessible regions (DARs); and generating an output comprising differentially expressed genes (DEG) and differential activity in domains of regulatory chromatin (DORC) for the one or more PBMCs to determine DEG transcriptional enrichment and DORC epigenetic enrichment, thereby characterizing PBMC transcriptional and epigenetic signatures.

In various embodiments, a system can be provided for characterizing transcriptional and epigenetic signatures in one or more PBMC based on transcriptomic and epigenomic analysis. The system can include a data store configured to store a set of single cell and/or bulk mRNA and ATACset data for one or more PBMC. The system can also include a computing device communicatively connected to the data store, including a multi-layer training engine configured to generate a trained multi-layer model for transcriptional and epigenetic signature characterization.

Other embodiments of these aspects include corresponding computer systems, apparatus, and computer programs recorded on one or more computer storage devices, each configured to perform the actions of the methods.

In the present disclosure, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

The practice of the present invention will employ, unless otherwise indicated, techniques of molecular biology, microbiology, cell biology, biochemistry, nucleic acid chemistry, and immunology. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual(3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference.

All publications mentioned herein are incorporated herein by reference for the purpose of describing and disclosing devices, compositions, formulations and methodologies which are described in the publication and which might be used in connection with the present disclosure.

The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

### II. Exemplary Descriptions of Terms

Unless otherwise defined, all terms of art, notations, and other scientific terms or terminology used herein are intended to have the meanings commonly understood by those of skill in the art to which this application pertains. Further, unless otherwise required by context, singular terms shall include pluralities and plural terms shall include the singular. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

In addition, as the terms “on”, “attached to”, “connected to”, “coupled to”, or similar words are used herein, one element (e.g., a material, a layer, a substrate, etc.) can be “on”, “attached to”, “connected to”, or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, the terms “comprise”, “comprises”, “comprising”, “contain”, “contains”, “containing”, “have”, “having”, “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein the specification, “a”, “an”, and “the,” may mean one or more. These terms generally refer to singular and plural references unless the context clearly dictates otherwise. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. “A and/or B” is used herein to include all of the following alternatives: “A”, “B”, “A or B”, and “A and B”.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, a “subject” or an “individual” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. A subject can be a mammal, preferably a human or humanized animal. The subject may be in need of prevention and/or treatment of a disease or disorder such as viral infection or cancer. The subject may have a viral infection, e.g., a coronavirus infection, or be predisposed to developing an infection. Subjects predisposed to developing an infection, or subjects who may be at elevated risk for contracting an infection (e.g., of coronavirus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g., subject resides in a densely-populated city or in close proximity to subjects having confirmed or suspected infections of a virus, or choice of employment, e.g. hospital worker, pharmaceutical researcher, traveler to infected area, or frequent flier.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

“Treating” or treatment of a disease or condition refers to executing a protocol, which may include administering one or more drugs to an individual, such as a patient (or subject), in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease, including COVID-19. In some embodiments, administering a therapeutically effective amount results in treating the condition to some degree.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

Similarly, the terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to stool, synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to stool, biopsy, blood and/or plasma. In some examples, biological samples include, but are not limited, to urine or stool. Biological samples include, but are not limited, to biopsy. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “marker” or “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, markers or biomarkers may be used for diagnostic purposes (e.g., to diagnose a health state, a disease state). The term “biomarker” can be used interchangeably with the term “marker.”

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O)ₙ).

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

As used herein, the term “functional assay” relates to an assay whereby a cell or cells is/are observed for functional behavior in vitro. This includes, for example, stimulation responsiveness (e.g. cytokine production), differentiation potential (e.g. colony forming assay), metabolism (e.g. measurements of oxygen consumption), migration (e.g. motility in migration assays), and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In various embodiments, the term “about” indicates the designated value ±up to 10%, up to ±5%, or up to ±1%.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

It should be understood that while deep learning may be discussed in conjunction with various embodiments herein, the various embodiments herein are not limited to being associated only with deep learning tools. As such, machine learning and/or artificial intelligence tools generally may be applicable as well. Moreover, the terms deep learning, machine learning, and artificial intelligence may even be used interchangeably in generally describing the various embodiments of systems, software and methods herein.

### III. Overview of Exemplary Workflow

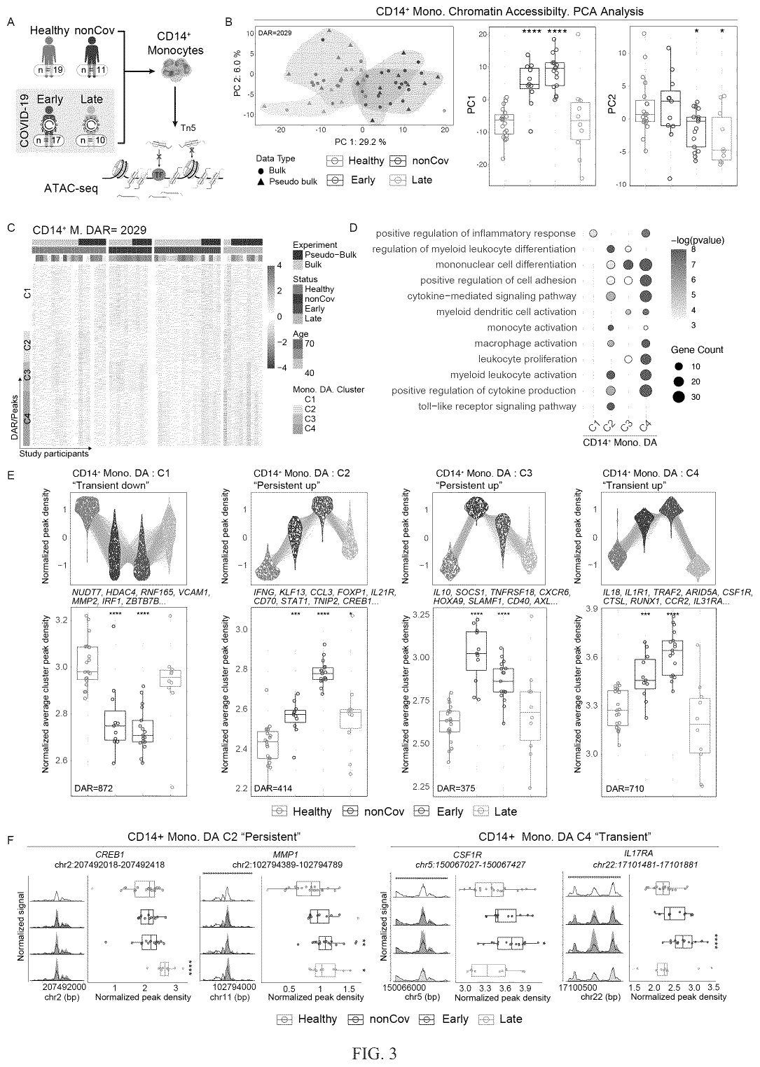

Exemplary workflows for various embodiments in accordance with the present invention, used for characterizing cellular molecular features and/or functional characteristics in an enriched population of rare circulating cells, including progenitor cells, from peripheral blood, such as for characterizing cellular molecular features and/or functional characteristics in accordance with various embodiments, are shown in FIGS. 3A, 5E, and 8A.

The workflow may include various operations including, for example, sample collection, sample intake, sample preparation and processing, data analysis, and output generation.

Sample collection may include, for example, obtaining a biological sample of one or more subjects. The biological sample may take the form of a specimen obtained via one or more sampling methods. The biological sample may be a peripheral blood sample, and or a PBMC sample. The biological sample may be obtained in any of a number of different ways. In various embodiments, the biological sample includes whole blood sample obtained via a blood draw. In various embodiments, the biological sample includes a cryopreserved whole blood sample or a cryopreserved PBMC sample. In other embodiments, the biological sample includes a set of aliquoted samples that includes, for example, a serum sample, a plasma sample, a blood cell (e.g., white blood cell (WBC), red blood cell (RBC)) sample, another type of sample, or a combination thereof. Biological samples may include nucleotides (e.g., ssDNA, dsDNA, RNA), organelles, amino acids, peptides, proteins, carbohydrates, glycoproteins, or any combination thereof.

Sample intake may include one or more various operations such as, for example, aliquoting, registering, processing, storing, thawing, and/or other types of operations.

Sample preparation and processing may include, for example, one or more operations to isolate and/or enrich one or more rare circulating cells, including progenitor cells. Sample preparation and processing can include, for example pooling multiple samples into a single assay tube and “demultiplexing” after analysis (in silico) based on individual subject genotype—genotype-based demultiplexing of single cell analysis. Employing these types of approaches provides a rapidly scalable and economic workflow for research-phase single cell dataset building for multiple diseases. Sample preparation and processing can also include working with a single sample in a single assay tube.

Further, sample preparation and processing may include, for example, data acquisition based on enriched rare circulating cells, including progenitor cells. For example, data acquisition may include use of, for example, but is not limited to, single nuclei (sn) RNA and assay for transposase-accessible chromatin (ATAC) sequencing (chromium single cell multiome ATAC+ gene expression) for PBMC, sorted PBMC subset “bulk” ATAC-seq, multiplexed immunoassay-based quantitation of plasma proteins, and/or immunophenotyping by flow cytometry.

Data analysis may include, for example, in depth transcriptomic and epigenomic analysis to identify differentially accessible regions. In some embodiments, data analysis also includes output generation. In other embodiments, output generation may be considered a separate operation from data analysis. Output generation may include, for example, generating final output based on the results of transcriptional enrichment and epigenetic enrichment. In various embodiments, final output may be used for determining the research, diagnosis, and/or treatment of a state associated with post-infection COVID-19.

In various embodiments, final output is comprised of one or more outputs. Final output may take various forms. For example, final output may be a report that includes, for example, a diagnosis output, a treatment output (e.g., a treatment design output, a treatment plan output, or combination thereof), analyzed data (e.g., relativized and normalized) or combination thereof. In some embodiments, the report can comprise a characterization of the cellular molecular features and/or functional characteristics and/or a characterization of transcriptional and epigenetic signatures. In some embodiments, final output may be sent to a remote system for processing. The remote system may include, for example, a computer system, a server, a processor, a cloud computing platform, cloud storage, a laptop, a tablet, a smartphone, some other type of mobile computing device, or a combination thereof.

In other embodiments, any workflow as described herein may optionally exclude one or more of the operations described herein and/or may optionally include one or more other steps or operations other than those described herein (e.g., in addition to and/or instead of those described herein). Accordingly, any workflow as described herein may be implemented in any of a number of different ways for use in the research, diagnosis, and/or treatment of, for example, post-infection COVID-19.

### IV. Characterization of Cellular Molecular Features and/or Functional Characteristics in Rare Circulating Cells to Identify Disease States and Therapeutic Targets

The invention described herein includes a breakthrough in blood stem cell biology that enables the discovery of stem cell disease states, including epigenetic scars, in rare circulating cells, such as HSPC, derived from peripheral blood (peripheral blood HSPCs, also termed pHSPC herein). In particular, the cellular molecular features and/or functional characteristics, such as transcriptional and epigenetic signatures, characterized from PBMCs can be derived from single-cell profiling of human pHPSCs post-SARS-CoV-2 infection. In addition, cell function assays on pHSPC can reveal functional changes, for example in post-SARS-CoV-2 infection.

HSPCs self renew and continuously generate immune cells. Thus, these cells hold the key for better understanding the body's long-term memory and response to inflammation and therapies. However, researchers are often “blind” to human HSPC dysregulation and plasticity, despite their obvious importance to disease. For example, pre-clinical and limited human studies show that inflammation can drive plasticity and disease states in HSPCs. In addition, it has been demonstrated that adjuvants and biologics can durably perturb or realign stem cell programs in animal models, but to extremely limited extent in humans. The first-in-class platform developed by the present inventors as described herein enables the characterization of pHSPC gene signatures, which can in turn allow for the identification of disease-associated HSPC programs and the effects of therapies on HSPCs and hematopoiesis.

At its root, medicine has historically been unable to comprehensively study the importance of rare circulating cells from blood, including progenitor cells, such as pHSPCs. In particular, few studies have focused on pHSPC dysregulation and plasticity, despite their obvious importance in most human diseases, due to the challenges in accessing and studying such cells in human patient populations. Thus, the full potential of understanding the biological importance of blood stem cells in human disease has heretofore been untapped. Further, the value of these approaches is highlighted by the role of stem cells at the root of the hematopoietic tree, as the origin of all blood cells, and given recent literature that indicates that pHSPC have plasticity, can influence and be influenced by disease states and can carry epigenetic memory that can persistently define the programs of mature progeny cells. Thus, the ability to study human pHSPC/blood stem cell biology, in a deep and meaningful way, using only peripheral blood is an enabling breakthrough.

As described herein, the present inventors discovered a process enabling the enrichment of rare circulating cells, including progenitor cells, captured from the circulating blood. In particular, enriched pHSPCs were found to recapitulate the diversity and programs of HSPC in bone marrow, by employing their innovative strategy described herein. Specifically, the present inventors therefore developed a unique strategy to enrich rare circulating cells, such as pHSPC, from blood and characterize their diversity at the single cell level; this strategy is called Peripheral Blood Mononuclear Cell analysis with Progenitor Input Enrichment (“PBMC-PIE”).

Analysis of peripheral blood-derived pHSPCs is of value for multiple reasons. First, HSPCs have widely acknowledged and considerable relevance to health and disease, since all blood cells derive from HSPCs, and since hematopoietic stem cells (HSCs) are self-renewing. Also, HSPCs are traditionally acquired through invasive and costly bone marrow biopsy, whereas peripheral blood is readily obtained. On account of these barriers to acquisition and study of HSPC, their analysis for understanding diverse disease has been limited to date.

With PBMC-PIE, the present inventors have characterized rare circulating cells, such as pHSPC, from blood at the single cell level, thereby allowing for the identification of a disease-specific pHSPC signature. This has been demonstrated herein in the context of post-infection COVID-19 (or “long COVID”). Further, this platform is amenable to identification of therapeutic targets for a disease once it has been characterized in rare circulating cells, such as the present inventors did with long COVID pHSPCs. As described herein, the platform revealed altered pHSPC phenotypes, indicating altered hematopoiesis, inflammatory and migratory properties of pHSPC and their progency monocytes, and characteristic features of long COVID. While pHSPC both replenish themselves and generate mature blood cells, the platform for analysis of pHSPCs together with mature PBMC that derive from them enables discovery of the source and cause of many mature blood cell phenotypes, for example, as the inventors demonstrate for pHSPC-derived phenotypes of inflammatory/migratory monocytes in post-COVID-19 patients. The platform detects pHSPCs which are durably altered (via persistent epigenetic memories) for at least one year; post-COVID-19 pHSPC memories can be blocked by therapeutic treatment.

pHSPC and Monocyte Alterations Following Severe COVID-19

pHSPC are long-lived self-renewing precursors to diverse mature immune cells (Seita and Weissman, 2010). As a result, they are endowed with the unique potential to serve as reservoirs of inflammation-induced epigenetic memory, which can result in altered hematopoiesis and phenotypes in innate immune cell progeny. Thus, we considered that acute viral infections, particularly SARS-CoV-2 and resultant severe COVID-19, characterized by systemic inflammation (Berlin et al., 2020; Chen et al., 2020; Guan et al., 2020; Holter et al., 2020; Huang et al., 2020; Wang et al., 2020a; Zhu et al., 2020), could trigger such a response.

We determined that rare circulating CD34⁺ pHSPC accurately capture the diversity and major transcriptomic and epigenomic signatures of bone marrow HSPC subsets by unprecedented deep single-cell profiling of paired blood and bone marrow samples from the same donors. Enrichment of circulating CD34⁺ pHSPC via PBMC-PIE (PBMC analysis with progenitor input enrichment), paired with single-cell combined ATAC/RNA-seq analysis, enabled deep molecular characterization of HSPC. Establishing this knowledge and these workflows opens broad opportunities to characterize the effects of diverse challenges and disease on human hematopoietic progenitor cells without invasive bone marrow biopsy collection.

This study uncovered coordinated and persistent epigenetic and transcriptional reprogramming of pHSPC and monocytes in convalescent COVID-19 patients, where these changes presumably contributed to distinct granulopoiesis, myelopoiesis, and inflammatory programs observed with disease and notably persisted long term (at least one year in our study). Interestingly, following COVID-19, pHSPC and monocytes share common distinguishing features including TF activities, and epigenomic and transcriptomic programs. These data indicate that COVID-19 induces changes in transcriptional and epigenetic programs of self-renewing multipotent HSC reservoirs, that these changes are durable over time and conveyed to newly produced monocytes (and potentially to other mature immune cells), and likely alter the tone of immune equilibrium and future responses.

These findings establish that natural infection can durably alter human adult stem cells in a manner that both distorts proportions of multilineage differentiation and conveys epigenetic programs from progenitor to progeny cells, influencing their phenotypes and responsiveness. This capacity for the human hematopoietic and innate immune system to encode a durable memory of SARS-CoV-2 infection highlights the need to understand similar phenotypes across a range of diverse disease, treatment, and vaccination contexts, and demonstrates the applicability of pHSPC in prognosis, diagnosis, and treatment for various disease states, as described elsewhere herein.

The findings that circulating CD34⁺ pHSPC accurately capture the diversity and major transcriptomic and epigenomic signatures of bone marrow HSPC subsets were used to study the long-lasting effects of COVID-19 on hematopoiesis and central trained immunity (Bekkering et al., 2021), focusing on assessment of epigenomic and transcriptional changes in circulating HSPC and immune cells. We found that severe COVID-19 induced long-lasting epigenetic alterations in pHSPC and their short-lived, circulating progeny monocytes. Transcription factor activity and chromatin accessibility in post-infection pHSPC were conveyed through differentiation to monocyte progeny, possibly rewiring gene expression programs.

These results establish a precedent for central trained immunity following viral infection in humans and indicate that recent observations of persistent monocyte epigenetic memory following influenza vaccination (Wimmers et al., 2021) can also derive from HSPC alterations. Beyond this epigenetic innate immune memory, we demonstrated that hematopoiesis is altered following COVID-19, with increases in myeloid and neutrophil progenitor populations and durable changes in transcription factor activities that direct these programs.

These results are the first-of-their-kind and are supported by the established characteristics of hematopoietic stem cells, capable of tuning their capacity to differentiate into alternative lineages in response to diverse non-homeostatic stimulation (Seita and Weissman, 2010). Increased myelopoiesis is noted as a surrogate clinical marker for infection or inflammation (Schultze et al. 2019), and is a well-recognized feature of trained immunity phenotypes (Netea et al., 2020). Increased myelopoiesis is also known to be involved in many pathologic conditions, such as inflammation in aging and atherosclerosis (Beerman et al., 2010; Murphy and Tall, 2016; Rohde et al., 2022; Schultze et al., 2019), and future studies may link these phenotypes to cumulative infectious and inflammatory challenges (Bogeska et al., 2022).

### Innate Immune Memory Following Severe SARS-CoV-2 Infection

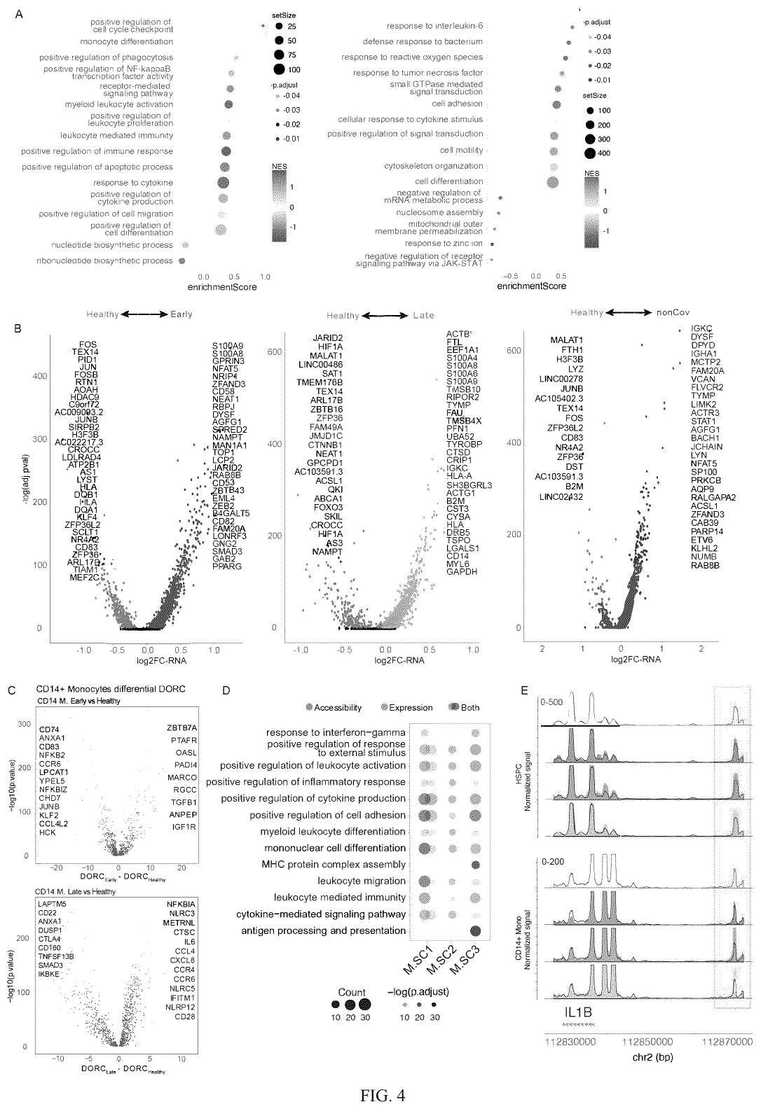

HSPC and monocyte programs following severe COVID-19 are complex, with individual cells bearing mixed inflammatory and interferon signatures and reduced expression of key negative feedback factors DUSP1 and NFKBIA. CD14⁺ monocytes in early convalescence (2-4 months post-acute COVID-19) feature epigenetic and transcriptional signatures of inflammation, likely with the prominent influence of NF-κB and AP-1 TFs. This active inflammatory CD14⁺ monocyte program resolves in late convalescence (4-12 months), though a distinct epigenetic monocyte phenotype persists, including increased chromatin accessibility at certain chemokines (e.g., CCL4 and CXCL8), chemokine receptors (e.g., CCR4, CCR6 and CXCR3), interferon stimulated genes (e.g., NLRC5, SOCS1, IFITM1), inflammatory genes (e.g., NFKBIA, S100A12, CTSC, IL6, CD28, NLRP12), and antigen presentation related genes (e.g., CD74, LGMN, HLA genes), though immunomodulatory genes were also primed (e.g., METRNL, NLRC3, KLF4, TNFAIP3) (FIG. 4C).

Post-COVID-19 monocytes appeared hyperresponsive with regard to IL-1b production following in vitro stimulation with viral infection-mimicking stimuli, indicating that these cells do indeed retain altered functional states. The identification of months-long altered monocyte programs, stemming from altered progenitor cells, following severe SARS-CoV-2 infection indicates that monocytes can contribute to chronic inflammation, either in affected tissues, via migratory and chemoattractant programs, or systemically. Indeed, elevated serum IL-1b was recently described as a correlate of PASC or “long COVID” (Schultheiß et al., 2022) potentially linking epigenetic monocyte phenotypes, IL-1b hyperresponsiveness, and PASC. Further studies can continue to define functional changes in the post-COVID-19 immune system and the full implications of the durable changes in HSPC and their progeny cells that we observed.

Persisting alterations in pHSPC and monocytes following severe SARS-CoV-2 infection indicate that long-term alterations in innate immune status can be a general feature of diverse infections. This dynamic aspect of blood development and innate immune memory can have major implications for vaccine responses and design, post-infectious inflammatory disease, and non-genetic variance in responses to infection. These results indicate that acute viral infections can induce months-long priming of anti-viral programs similar to what has been described as “anti-viral resilience” following vaccination against influenza (Wimmers et al., 2021). As a corollary, in the winter of 2020-2021, the aberrantly low frequency of non-SARS-CoV-2 respiratory infections (and resulting lack of primed anti-viral programs) may have led to subsequent increased susceptibility to pathogenic viral infections and the unusual epidemic of respiratory syncytial virus and rhinovirus in the summer of 2021 (Agha and Avner, 2021).

### Transcription Factor Programs Underlie Post-COVID-19 Phenotypes

Transcriptional and epigenetic changes in pHSPC and monocytes occur following COVID-19 alter cell differentiation, function, and response to stimulation. These results indicate that altered TF activity in post-COVID-19 pHSPC can have two independent effects, (i) quantitatively altering immune system composition through biasing lineage selection in HSC/MPP to increase frequencies of myeloid and granulocyte progenitors, and (ii) qualitatively changing mature immune cell responsiveness and phenotypes through establishing heritable chromatin accessibility or poised transcriptional states at select inflammatory or immunomodulatory genes. Specific TF networks can adapt to program both outcomes in parallel. For example, inflammation responsive transcription factors NFKB1, NFKB2, CEBPB, and AP-1 family members, can both prime genes like IL1B for more rapid, higher-level induction in mature monocytes, and also promote GMP differentiation in HSPC (de Laval et al., 2020).

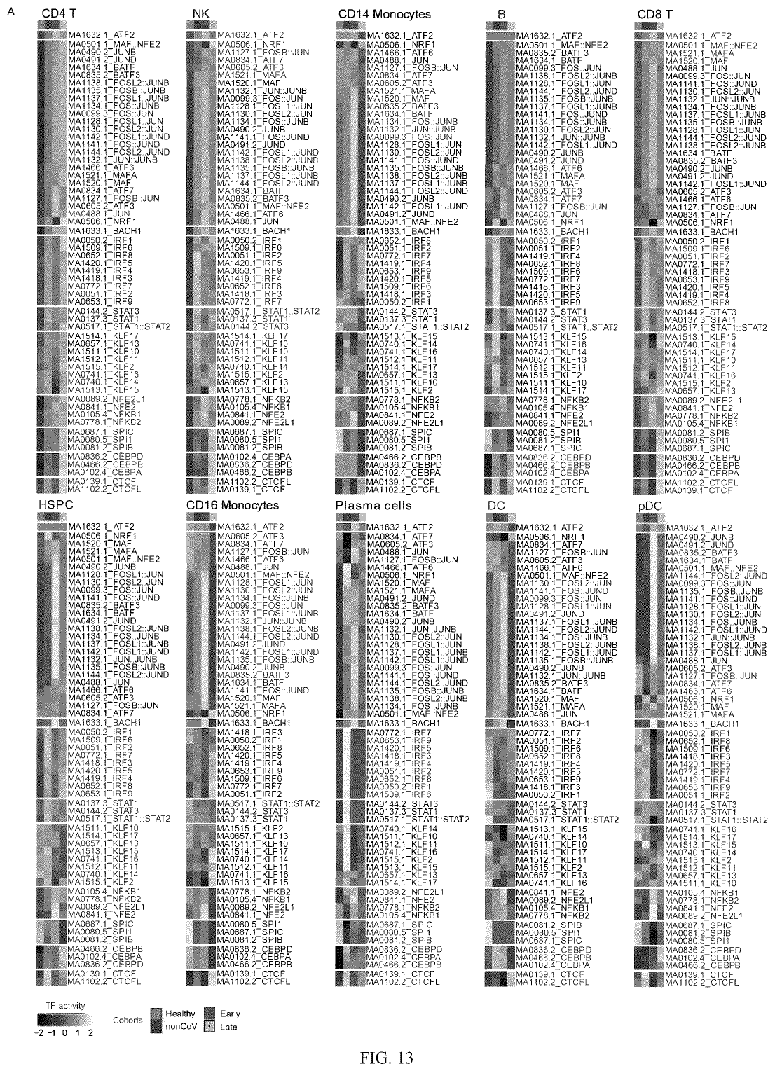

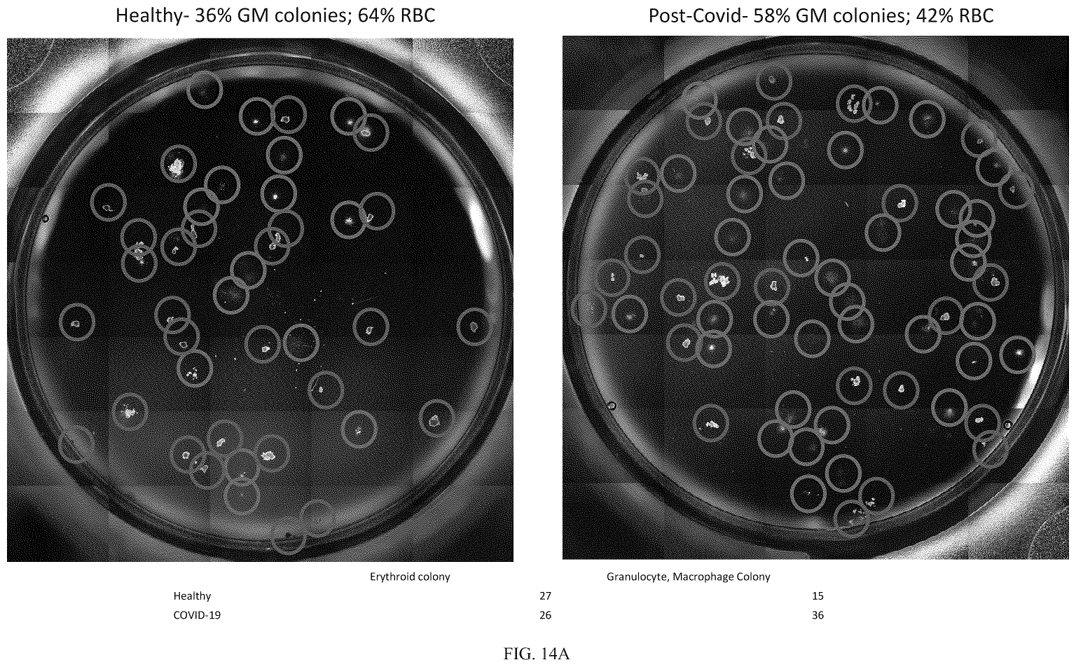

Consistent with heritable features conveyed from stem cells to progeny cells, we observed distinguishing post-COVID-19 transcriptional programs enriched in both HSPC and monocytes. Both CEBP TFs (de Laval et al., 2020) and AP-1 TFs (Larsen et al., 2021) have been reported to sustain durable epigenetic memory in adult stem cell populations. Notably, the AP-1 family is subject to complex regulation following COVID-19, with elevated activities across cell types in early convalescence (in response to lingering inflammation) followed by negative feedback regulation of AP-1 genes and diminished activity in late convalescence in many cell types (FIG. 13). However, HSPC and a few other cell types (e.g., T, B, and NK cells) maintained higher than baseline levels of AP-1 chromatin binding for at least one year post COVID-19. Intriguingly, it was recently demonstrated that AP-1 activity can maintain increased chromatin accessibility in inflammation experienced adult stem cells (epidermal) contributing to augmented ability of these stem cells to respond to tissue damage (Larsen et al., 2021), providing a precedent for the potential role of AP-1 in durable alterations of human HSPC following COVID-19.

One durable feature of post-COVID-19 pHSPC and monocytes was increased IRF activity. Notably, this is similar to what has been described in monocytes following adjuvanted influenza vaccine (H5N1+AS03), which provided a degree of heterologous anti-viral protection to Zika and Dengue viruses (Wimmers et al., 2021). Future studies should address the possibility that the persistent IRF activity following severe disease may represent a primed rather than active anti-viral program. Indeed, IRF factors interact with BAF complex (SWI/SNF) chromatin remodelers to maintain open or poised chromatin states and to drive active transcription (Song et al., 2021). IRF1 activity, which drives active inflammation-responsive interferon-stimulated gene transcription, was reduced post-COVID-19, but several other IRF factors were increased, including IRF2 and IRF3, which have been shown to interact with the BAF complex to retain ISG in a poised state (Ren et al., 2015). Persisting IRF chromatin binding activity post COVID-19 can therefore result in increased poising and responsiveness of IRF target genes, in part through the maintenance of accessibility via IRF-BAF complex interactions.

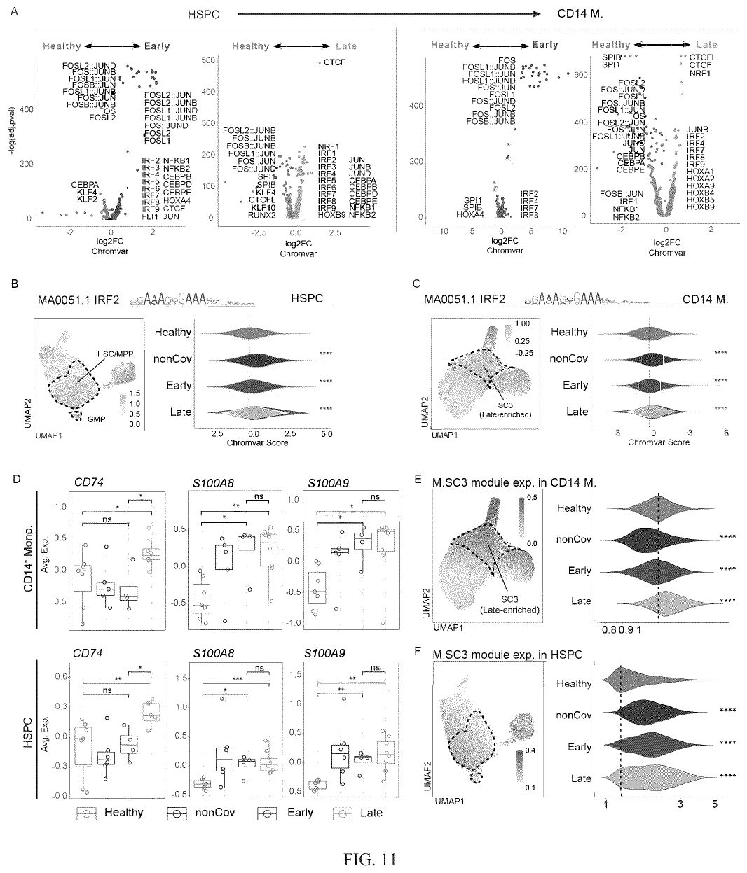

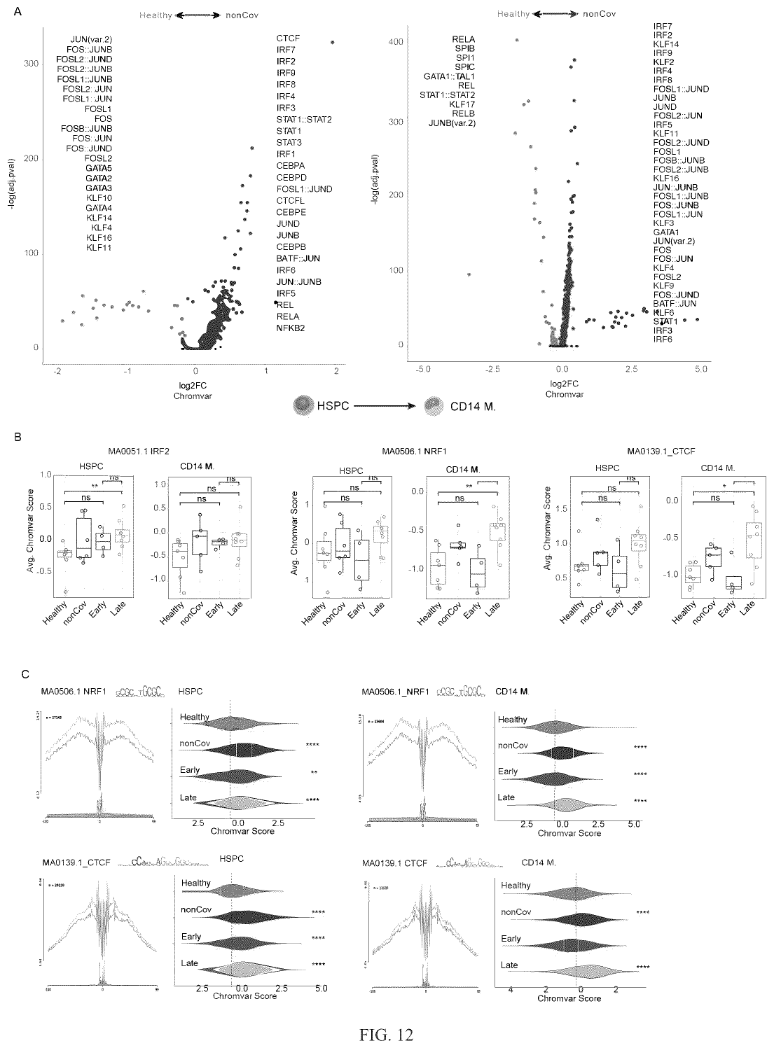

Another mechanism that can poise chromatin at select loci in post-COVID-19 monocytes involves the increased activity of CCCTC-binding factor (CTCF) (FIG. 11A, FIG. 12B-C). CTCF is a ubiquitous transcription factor with multifaceted activities and is one of the core architectural proteins that construct the three-dimensional organization of the eukaryotic genome. CTCF also plays a specific and important role in the differentiation of post-mitotic human monocytes into dendritic cells or macrophages, which rely on CTCF-cohesin-regulated chromatin looping in the absence of cell-cycle dependent chromatin reorganization to induce transcriptional programs of differentiation and activation (Minderjahn et al., 2022). This direct activity of CTCF in monocyte differentiation can explain our observations in post-COVID-19 monocytes (FIG. 11A, FIG. 12B), namely the paired enrichment of both CTCF activity and epigenetic and transcriptional signatures of macrophage and dendritic cell differentiation. This acquisition and retention of an epigenetic and transcriptional program resembling those of more mature cells can render these cells more responsive to cues for activation, migration, and cell-cell communication.

### Mechanisms Contributing to Long-Term Sequelae Following COVID-19

Understanding the long-lasting effects of COVID-19-associated inflammation on hematopoiesis and innate immune memory is relevant for the hundreds of millions of people worldwide who have recovered from COVID-19 and perhaps especially for those experiencing long-term clinical sequelae of COVID-19 (Del Rio et al., 2020; Xie et al., 2021), as persistent symptoms could be influenced by alterations in immune activity and hematopoiesis. Beyond post-acute sequelae of SARS-CoV-2 infection (PASC), the etiology of incomplete recovery after critical illness, including post-ICU syndrome (PICS) (Jaffri and Jaffri, 2020), is poorly understood. Lasting alterations in pHSPC, immune cells, and their inflammatory response programs can contribute to incomplete recovery in these contexts.

Our study indicates potential molecular mechanisms that can contribute to long-term sequelae of inflammation, including (i) persisting monocyte transcriptional and epigenetic programs of activation, differentiation, migration, and antigen presentation, with linked underlying pHSPC epigenetic programs, that may continue to fuel inflammation and fibrosis, especially in tissues with residual inflammation (e.g. lung and upper respiratory mucosa post COVID-19); and (ii) altered hematopoiesis including increased myeloid and neutrophil progenitors whose progeny can contribute to ongoing inflammation. Based upon neutrophil stimulating CSF-3 (G-CSF) production by lung epithelium in response to SARS-CoV-2 infection (Fang et al., 2020) and persistent CSF-3R upregulation in post-COVID-19 pHSPC along with other neutrophil related genes (e.g., S100A9, S100A8, CAMK1D, CD74) (FIG. 10C), these data show that feedforward loops (such as a CSF-3/CSF-3R) can contribute to GMP-driven pathology in both acute and chronic COVID-19 diseases. Our study design and cohort size were suitable for the detection of post-COVID-19 programs that are more uniformly altered after COVID-19 and severe disease. Further studies that follow long-term outcomes can more sensitively parse the molecular features that associate with the vast range of unexplained long-term sequelae after severe (and sometimes mild) illness, including post-acute sequelae of SARS-CoV-2 infection (PASC) (Del Rio et al., 2020; Huang et al., 2021; Ramakrishnan et al., 2021) and post-ICU syndrome (PICS) (Kotfis et al., 2020).

### Rare Circulating Cell Phenotypes and Disease

While this study focuses on blood cells, it is important to point out that diverse other cell types have been demonstrated to harbor epigenetic memory (Friščić cid et al., 2021; Naik et al., 2017; Niec et al., 2021). Particularly when they reside in affected tissues, these cells may change in their frequencies, differentiation programs, and phenotypes, and also retain epigenetic memory of anti-viral inflammation with important and enduring influence on tissue defense or sequelae (Niec et al., 2021; Ordovas-Montanes et al., 2020). Importantly, enrichment of rare circulating progenitor cells and establishment of matched bone marrow and peripheral HSPC phenotypes was a critical advance enabling evaluation of pHSPC together with their progeny immune cells from peripheral blood samples in this study. Extending this approach to diverse tissues (particularly those with resident stem and progenitor cells, e.g., intestinal epithelium) and disorders (hematologic disease, malignancy, inflammation, and infection) can unveil epigenetic and progenitor-based mechanisms of pathogenesis and inform therapeutic strategies.

### V. Use of Stem Cells Derived from Peripheral Blood in Place of Stem Cells Derived from Bone Marrow

As described in the preceding section, the present inventors have built a unique dataset of peripheral blood-derived HSPC (pHSPC) that includes transcriptomic and epigenomic single cell data. These data have been found to capture the diversity of the bone marrow HSPC subsets, thereby indicating that pHSPC can serve as a surrogate or replacement for analysis of bone marrow derived HSPC.

A bone marrow exam is an umbrella term used to refer to both bone marrow aspiration and bone marrow biopsy. Both of these procedures are performed to collect and examine the bone marrow in order to diagnose and monitor certain human diseases. Both procedures involve insertion of a needle into the pelvic region and removal of a liquid portion of the bone marrow (bone marrow aspiration) and/or a small piece of bone tissue (bone marrow biopsy). Bone marrow aspiration is sometimes performed alone, as it is the less invasive of the two, however it's usually combined with a bone marrow biopsy for a comprehensive bone marrow exam.

Bone marrow examinations are used for many human diseases, however some of the most common include: inflammatory diseases or conditions (including fevers of unknown origin), types of cancer (e.g. lymphoma, leukemia, myeloma, metastatic cancers, etc.), metabolic diseases or conditions (e.g. anemia, hemochromatosis, etc.), blood disorders or conditions (e.g. leukopenia, leukocytosis, thrombocytopenia, thrombocytosis, pancytopenia, polycythemia, etc.).

Bone marrow examinations are costly and uncomfortable for the patient. In addition, while bone marrow examinations (both aspirations and biopsies) are typically performed on an outpatient basis with little to no special preparation required, there are cases where sedatives are required as well as several rare, but potential, risks. These include excessive bleeding, infection at the needle insertion site, long-lasting pain and discomfort at the bone marrow exam site, and penetration of the breastbone leading to heart and/or lung problems (during sternal bone marrow exams only).

From start to finish, bone marrow examinations typically only last an average of 30 minutes (more if sedation is required) and do not require a hospital stay. However, the pain, swelling, and discomfort which often occur at the procedure site can persist for several days, and patients activities are impacted in the days after the procedure, as they are instructed to keep the procedure site dry and to avoid strenuous activities/exercise for at least 24 hours. While relatively minor, these aspects of the recovery from bone marrow examinations can still be disruptive to a patient's everyday life.

Using the PBMC-PIE as described herein to characterize stem cells derived from peripheral blood, as opposed to bone marrow, will largely avoid these issues due to i) the lack of a sedation requirement; and ii) the less invasive nature of blood draws. PBMC-PIE requires only a routine blood draw and thus largely avoids the pain, risk, and recovery issues associated with bone marrow examinations.

Additionally, as compared to bone marrow examinations, which can carry a high cost (>$1,000 USD) and must be performed by a specialist doctor (hematologist-oncologist) or nurse with special training, PBMC-PIE can be performed with significantly reduced medical care costs and facility requirements. Further, while the results of a bone marrow examination will depend on the type of subsequent laboratory tests and the nature of the patient's disease status, as mentioned previously, PBMC-PIE can recapitulate these test results through its ability to quantify and characterize the hematopoietic stem cell compartment in both the blood and bone marrow from a human blood sample. In sum, as compared to conventional bone marrow examinations (both aspirations & biopsies) PBMC-PIE has fewer risks, a faster recovery, can be used for the same types of laboratory tests, and has the distinct advantage of being able to be combined with other routine blood draws.

### VI. Computer Implemented System

In various embodiments, the systems and methods for characterizing cellular molecular features and/or functional characteristics in an enriched population of rare circulating cells, including progenitor cells, from peripheral blood can be implemented via computer software or hardware.

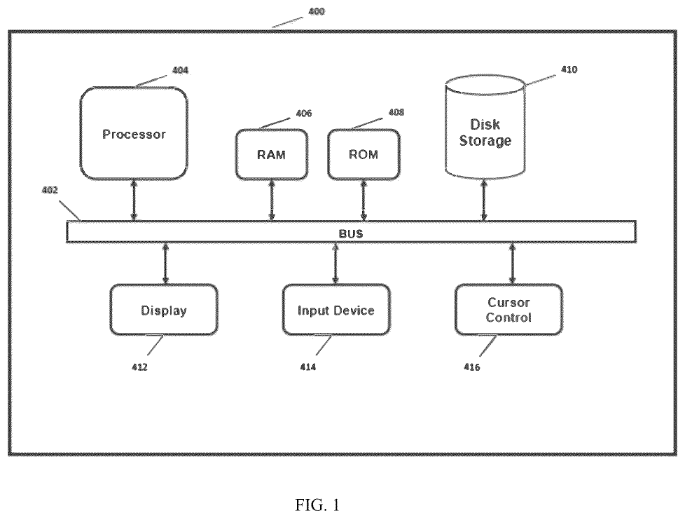

FIG. 1 is a block diagram illustrating a computer system 100 upon which embodiments of the present teachings may be implemented. In various embodiments of the present teachings, computer system 100 can include a bus 102 or other communication mechanism for communicating information and a processor 104 coupled with bus 102 for processing information. In various embodiments, computer system 100 can also include a memory, which can be a random-access memory (RAM) 106 or other dynamic storage device, coupled to bus 102 for determining instructions to be executed by processor 104. Memory can also be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communication of information and command selections to processor 104. Another type of user input device is a cursor control 116, such as a mouse, a trackball or cursor direction keys for communicating direction information and command selections to processor 104 and for controlling cursor movement on display 112. This input device 114 typically has two degrees of freedom in two axes, a first axis (i.e., x) and a second axis (i.e., y), that allows the device to specify positions in a plane. However, it should be understood that input devices 114 allowing for 3-dimensional (x, y and z) cursor movement are also contemplated herein.

Consistent with certain implementations of the present teachings, results can be provided by computer system 100 in response to processor 104 executing one or more sequences of one or more instructions contained in memory 106. Such instructions can be read into memory 106 from another computer-readable medium or computer-readable storage medium, such as storage device 110. Execution of the sequences of instructions contained in memory 106 can cause processor 104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 104 for execution. Such a medium can take many forms, including but not limited to, non-volatile media, volatile media, and transmission media. Examples of non-volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 104 of computer system 100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 100 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 100, whereby processor 104 would execute the analyses and determinations provided by these engines, subject to instructions provided by any one of, or a combination of, memory components 106/108/110 and user input provided via input device 114.

Although specific embodiments and applications of the disclosure have been described in this specification, these embodiments and applications are exemplary only, and many variations are possible. Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### VII. Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

Cellular samples and assays for peripheral hematopoietic stem and progenitor cell (pHSPC) enrichment and characterization

The cellular samples and assays used in Examples 2-12 are described below.

### Study Cohort

A total of 168 study participants were enrolled at Weill-Cornell Medicine/New York-Presbyterian Hospital between March 2020 and March 2021. Participants were recruited from the inpatient division of New York-Presbyterian Hospital and the Weill-Cornell Medicine pulmonary and post-ICU clinics. No statistical methods were used to predetermine sample size. COVID-19 severity scoring was based on the COVID-19 World Health Organization (WHO) Severity Classification [https://www.who.int/blueprint/priority-diseases/key-action/COVID-19_Treatment_Trial_Design_Master_Protocol_synopsis_Final_18022020.pdf].

Subjects were binned into the following groups:
i) healthy volunteer donors;
ii) recovered mild COVID-19 patients (WHO score 1-2);
iii) recovered severe COVID-19 patients (WHO score 6-7); and
iv) recovered non-COVID-19 critically ill patients.

Inclusion criteria for each group were as follows:
i) healthy volunteer donors: absence of clinical COVID-19 symptoms at any time prior to blood collection (prior negative SARS-CoV2 PCR and/or seronegative status also considered when available);
ii) recovered mild COVID-19 patients: PCR-proven SARS-CoV2 infection with the presence of clinical COVID-19 symptoms not requiring hospitalization;
iii) severe COVID-19 patients: PCR-proven SARS-CoV2 infection with the presence of clinical COVID-19 symptoms requiring admission to ICU-level care and the use of mechanical ventilation; and
iv) recovered non-COVID19 critically ill patients: absence of SARS-CoV2 infection as measured by PCR and negative serology on admission and/or throughout hospital admission and non-COVID-19 related critical illness requiring admission to the medical, neurological, or cardiology intensive care unit.

Prior infection status in healthy volunteer donors and recovered mild COVID-19 groups was confirmed by SARS-CoV2 serological testing after donation. There were no specific exclusion criteria other than an inability to provide informed consent or SARS-CoV2 positive serology in asymptomatic healthy volunteers (asymptomatic infection) and non-COVID19 critically ill participants. Blood was collected in EDTA or sodium heparin-coated vacutainers and kept on gentle agitation until processing, and all blood was processed on the day of collection. Age, sex, and comorbidity data were obtained through EPIC EHR records or when unavailable through a standardized form at the time of donation.

### PBMC and Plasma Isolation

Whole blood from EDTA or Heparin tubes (BD 366643 and BD 368480, respectively) was spun at 500 g for 10 minutes at room temperature with no brake. The undiluted plasma was aliquoted to 1.5 ml microcentrifuge tubes and stored at −80° C. for subsequent analysis.

After removal of plasma, the blood was mixed at a 1:1 ratio with room temperature RPMI medium (Corning 10-040-CM), layered over Ficoll-Paque PLUS (GE 17144002), and spun at 700 g for 30 minutes at room temperature with minimum acceleration and no brake. The PBMC layer was isolated and washed with RPMI. Cells were then treated with ACK lysis buffer for 3 minutes and counted on a Countess 2 automated cell counter (Thermo Fisher AMQAX1000). Cells were centrifuged again and resuspended in freezing medium (90% FBS+10% DMSO) and stored in cryogenic vials in a freezing container (Thermo Fisher 5100-0001) at −80° C.

### Paired BMMC and PBMC Acquisition

BMMC and PBMC were freshly isolated from the same two adult donors recruited by AllCells (Alameda, CA). The donors gave written consent in accordance with protocols approved by their governing IRB. The isolated BMMC and PBMC were cryopreserved as PMBC from our cohort.

### CD34+ and CD14+ Cell Isolation

Frozen PBMC and BMMC were thawed in a 37° C. water bath, washed with RPMI, and centrifuged. An aliquot of PBMCs was stained with 7-AAD (Biolegend 420404, 1:20) alone. The rest of the cells were incubated with CD34 microbeads (Miltenyi 130-046-702) and isolated by placing them on a magnetic column (Miltenyi 130-042-201) as per the manufacturer's specifications. The positive fraction obtained from the magnetic column was stained with the following antibodies—CD34-FITC (Miltenyi 130-113-178, 1:100), CD49f-Pacific Blue (Biolegend 313620, 1:200), CD90-PE (Biolegend 328110, 1:100), CD38-PE/cy7 (Biolegend 303516, 1:100), CD45RA-APC/cy7 (Biolegend 304128, 1:400), Lineage markers (CD20-Biotin (Biolegend 302350, 1:100), CD16-Biotin (Biolegend 302004, 1:100), CD3-Biotin (Biolegend 344820, 1:100), CD56-Biotin (Biolegend 362536, 1:100), and CD14-Biotin {Biolegend 301826, 1:100), and 7-AAD (Biolegend 420404, 1:20). After incubating in the dark for 30 minutes, cells were washed with PBS and incubated with Streptavidin-BV605 (BD 563260, 1:500) for an additional 30 minutes. CD34+ cells from the positive fraction and viable PBMC from the PBMC aliquot were then sorted on a BD FACSAria cell sorter and mixed at 1:5-1:20 ratios.

The negative fraction from the magnetic column was stained with the following antibodies—CD14− APC (BD 340436, 1:1000), CD8-FITC (Biolegend 300906, 1:400), and 7-AAD (Biolegend 420404, 1:20). After incubating in the dark for 30 minutes, cells were washed with PBS, and CD14+ cells were sorted on a BD FACSAria cell sorter.

ATAC-Seq CD34+ pHSPC and CD14+ Monocytes

To perform ATAC-seq, the Omni-ATAC-seq protocol was followed (Corces et al., 2017). We used 50,000 cells for CD14+ monocytes, and 3000˜5000 for CD34+ pHSPC. pHSPC were sorted directly into the PCR tubes prior to following Omni-ATAC-seq protocol.

### Single-Cell Assays

Nuclei were isolated from a mix of CD34+ cells and PBMC (or BMMC) according to ‘Low Cell Input Nuclei Isolation’ protocol (10× Genomics CG000365-Rev B) and were processed using Chromium Controller & Next GEM Accessory Kit (10× Genomics 1000202) and Chromium Next GEM Single Cell Multiome ATAC+Gene Expression Reagent Bundle (10× Genomics 1000285) following the manufacturer's User Guide (10× Genomics CG000338-Rev D). Targeted nuclei recovery ranged from 5,000 to 10,000. The single-cell RNA and ATAC sequencing libraries were prepared using Dual Index Kit TT Set A (10× Genomics 1000215) and Single Index Kit N Set A (10× Genomics 1000212) respectively and sequenced on Illumina NovaSeq6000 or NextSeq platform.

### Plasma Protein Analyses

The frozen plasma was thawed, aliquoted, and either analyzed immediately or re-frozen and shipped for analysis. All analysis was performed on samples after their first freeze-thaw cycle.

### Cytokine Analysis

Plasma was shipped to Eve Technologies for their 15-plex human pro-inflammatory cytokine assay (Eve Technologies, Calgary, AB, Canada). All samples were analyzed in duplicate.

### Antibody Assay

The SARS-CoV-2 total RBD antibody (TAb), surrogate neutralizing antibody (SNAb), and avidity were used to measure plasma antibody levels on the TOP-Plus (Pylon 3D analyzer; ET Healthcare) as previously described (Racine-Brzostek et al., 2021).

### Example 2

### Analytical Methods for Enriched pHSPC

The data analysis methods used in Examples 3-11 are described below.

### Bulk ATAC-Seq Data Processing

ATAC-seq paired-end sequences (n=54 for sorted CD14+, n=47 for sorted CD34+) were trimmed using trimmomatic (Bolger et al., 2014) and the trimmed reads were aligned to the GRCh38 (hg38) genome using bwa (Li, 2013). These numbers include convalescent COVID-19 groups with mild symptoms. However, samples from these groups were excluded from differential analysis. Further processing steps were carried out on the aligned data using Picard (http://broadinstitute.github.io/picard/) and Samtools (Li et al., 2009). MACS2 (Zhang et al., 2008) was used for peak calling on the processed data using the BAMPE option with default parameters. For quality control, FRiP scores were calculated for all samples using the featurecounts program available in the Subread package (Liao et al., 2014). Samples with low FRiP scores (<0.15) were removed from downstream analysis. To ensure that the samples being removed were indeed poor quality, we conducted visual inspections using IGV (Robinson et al., 2011). After FRiP score filtering, we were left with 39 CD14+ samples and 18 CD34+ samples, including Mild groups.

### Single-Cell ATAC-Seq Data Processing

Two single-cell ATAC-seq samples were preprocessed using the Cell Ranger ATAC 1.2.0 pipeline and aligned to the GRCh38 (hg38) genome. The cellranger output was processed using Signac (Stuart et al., 2021). Individual samples were filtered out. Amulet (Thibodeau et al., 2021) was used for filtering out doublets. Post QC and doublet removal, the remaining steps of the Signac pipeline (TF-IDF normalization, SVD, UMAP embedding, and clustering) were completed. UMAP embedding and clustering were done using 30 PCs. The cells were annotated by using a reference PBMC scRNA-seq dataset with Seurat's anchor transfer functions.

### Multiome Data Processing

The Multiome data (ATAC+RNA) (n=30 for the study cohort, and n=2 for paired BMMC and PBMC data) were preprocessed using the Cell Ranger ARC 1.0.0 pipeline and aligned to the hg38 genome. The cellranger output was then processed using the Seurat Weighted Nearest Neighbor Pipeline (Hao et al., 2021). Low QC cells were filtered out. Doublets were removed using Amulet (Thibodeau et al., 2021) for snATAC-seq and Scrublet (Wolock et al., 2019) for snRNA-seq. Initial annotations of cells were carried out using the reference PBMC CITE-seq data in the Seurat package.

The Multiome data from different individuals were pooled using Seurat and Signac for snRNA-seq and snATAC-seq, respectively. Three samples were excluded from the pooling process due to poor quality based on the initial clustering. Low QC cells from individual samples were filtered out from the pooled data. For the snRNA-seq object, sctransform normalization was applied, followed by PCA, and 30 PCs were used for UMAP embedding and clustering. The merged dataset was also batch corrected using Harmony (Korsunsky et al., 2019) with all samples being used as a batch, and the UMAP embedding and clustering were repeated using 20 PCs. The snRNA-seq object was then annotated using the reference PBMC CITE-seq object in Seurat.

We then pooled snATAC-seq profiles from all samples and did the first round of cell-type annotation based on snRNA-seq annotations. Then we called peaks on each cell type using MACS2 (version 2.1.2) with the following parameters: ‘callpeak--nomodel--nolambda--keep-dup all--call-summits.’ Peak summits from all cell types were combined, extended on both sides by 150 bp. Redundant peaks were removed based on the q-value from MACS2. Using the generated peak region list, the number of reads overlapping a given peak window was determined for each unique cell barcode tag. This generated a peak by cell counts matrix corresponding to ATAC reads in peaks for each cell profiled. High-quality cells are retained with a fraction of reads in peaks (FRiP)>0.4 and sequencing depth >1000. The cells filtered out in this step were also removed from the snRNA-seq object to ensure the same cells were retained across both modalities.

After QC, the snRNA-seq object was reprocessed using sctranform, PCA, clustering, UMAP, Harmony, and PBMC CITE-seq reference annotation. In the end, 23 clusters were obtained from the snRNA-seq data using default parameters of Seurat (30 PCs for PCA). Annotations of these clusters were finalized based on the expression of marker genes for distinct immune cell types. 2 of these clusters were labeled as potential doublets and removed from downstream analyses since they expressed marker genes of more than one immune cell type. In the end, 21 clusters were retained that included 197,260 cells out of 260K cells in total in the snRNA-seq object. The same cells were then retained in the snATAC-seq component of the Multiome data, and the annotations were transferred. Once the cell type annotations for major cell types are finalized, we repeated the peak calling using MACS2 and the aforementioned parameter settings, which are used in the downstream DORC analyses using the ArchR (Granja et al., 2021) pipeline. The merged snATAC-seq object was also processed using the Signac pipeline with TF-IDF normalization, SVD, UMAP embedding, and clustering. We used Harmony (as part of the Signac pipeline) for batch correction in snATAC-seq data using individual samples as a batch. For both non-corrected and Harmony-batch-corrected UMAP embeddings and clustering, 30 PCs were used.

pHSPC Annotations

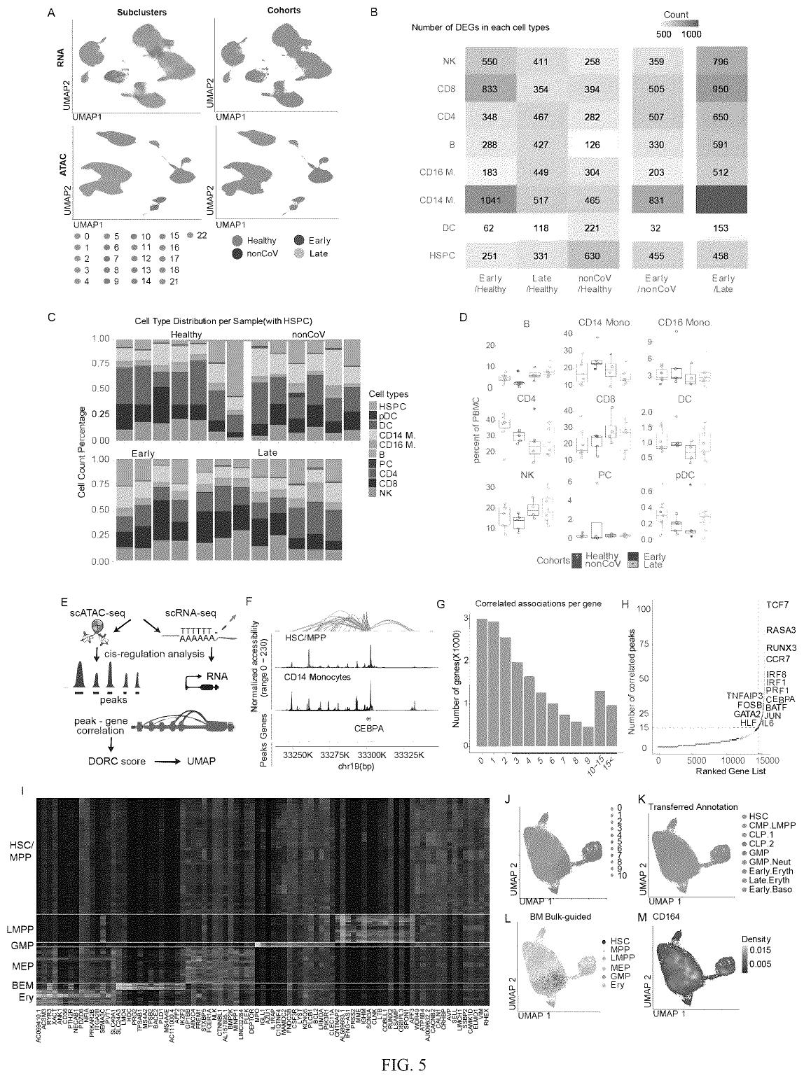

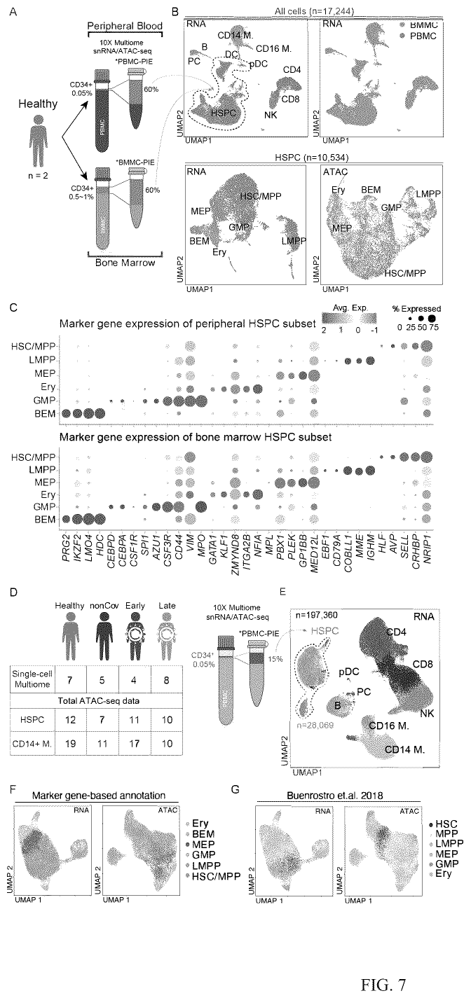

An unbiased workflow was used to cluster our 28,069 peripheral CD34+ pHSPC and identify differentially expressed genes among the 10 resulting subclusters (FIG. 5J). Manual curation of subclusters using marker genes from the literature (Buenrostro et al., 2018; Granja et al., 2019; Pellin et al., 2019) led us to merge select subclusters resulting in six pHSPC subsets defined by both well-characterized and novel marker genes (FIG. 7B-C, FIG. 5I).

While subclusters of snRNA-seq pHSPC data were annotated based on the expression of manually curated marker genes in each cluster, the snATAC-seq data was separately annotated for the pHSPC subtypes using a previously reported bulk-guided approach (Buenrostro et al., 2018). Briefly, using the bulk ATAC-seq peak set, we generated a peak by cell counts matrix. We identified 27 principal components (PCs) of variation in reference bulk ATAC-seq samples, then scored every single cell by the contribution of each PC. Cells were subsequently clustered using the Euclidean distance between these normalized single-cell PCs scores and PCs of bulk samples. The annotations based on the bulk ATAC-seq dataset were then transferred to the snRNA-seq object based on identical cellular identities. To transfer pHSPC subtype annotations from public data to our pHSPC data, we used public scRNA-seq data of BMMC from healthy participants and the Seurat package (Hao et al., 2021). The data available from GEO with access code GSE139369. Seurat object was created using BMMC data, normalized, then anchors for transferring annotation was defined by FindTransferAnchors( ) function of Seurat package using BMMC data as a reference. BMMC annotation information was transferred via anchors to our data using TransferData( ).

snATAC-seq Motif Analysis

Motif enrichment in the pooled snATAC-seq dataset was conducted using two methods. Before conducting the enrichment analysis, motif information was added to the pooled object using the AddMotifs function in Signac (Stuart et al., 2021). Motif information for hg38 was added to the object from the JASPAR2020 database.

For the first approach, we calculated overrepresented motifs in a set of differentially accessible peaks. Differential accessibility (DA) analyses were carried out using the pooled single-cell ATAC object (n=32) using Seurat's FindMarkers function (Hao et al., 2021) by conducting pairwise comparisons among clinical groups for each cell type. The normalized ATAC counts matrix was used for DA analyses using a logistic regression framework with a minimum fold change value of 0.25 and minimum percentage of cells for feature detection at 10%. Differential peaks with an adjusted p-value <0.05 were kept. We then used these top differential peaks to find overrepresented motifs using the FindMotifs function, which uses a hypergeometric test to find overrepresented motifs in a set of genomic features.

To visualize the chromVAR score as a heatmap, we first took the mean of each TF chromVAR score for each cell. Then median was taken for each group, followed by Z-score normalization. We used select TF families to visualize the heatmap.

Footprinting was also performed for a smaller select set of transcription factors using the Footprint function in Signac. This calculated the footprinting information of the motifs for every instance in the genome using the whole genome (Hg38) as a background.

### Peak-Gene Cis-Association and DORC Identification

To calculate peak-gene associations, the previously published approach was used (Ma et al., 2020). We considered all the peaks that are located in the +/−50 kb window around annotated TSSs. We used peak counts and imputed gene expression to calculate the observed Spearman correlation (obs) of each peak-gene pair. To estimate the background, we generated 100 background peaks for each peak by matching accessibility and GC content (chromVAR) (Schep et al., 2017) and calculated the Spearman correlation coefficient between those background peaks and the gene, resulting in a null peak-gene Spearman correlation distribution. We then calculated the expected population mean (pop.mean) and expected population standard deviation (pop.sd) from expected Spearman correlations. The Z score is calculated by z=(obs-pop.mean)/pop.sd. For peaks associated with multiple genes, we only kept peak-gene associations with the smallest p-value.

To define DORCs, we selected genes with at least 8 peaks per gene. The DORC score was calculated at each DORC gene for each cell. We defined the DORC score by summing up all the significantly correlated peak counts per gene per cell. We then normalize the DORC score by dividing the DORC score by the total unique fragments in peaks and obtain a cell×DORC score matrix.

### Visualizing Clinical Group Density on UMAP Plots

With the processed Seurat RNA-seq object, we extracted shared nearest neighbor graph information (Hao et al., 2021). Using this data, we determined the 50 nearest cells of each cell, then calculated the frequency of each group. We then included group frequency information of each cell in the metadata to visualize it on a UMAP plot using Nebulosa (Alquicira-Hernandez and Powell, 2021).

### UMAP Visualization

UMAP plots to show clusters, groups, samples, and annotations were generated using DimPloto function in the Seurat package (Hao et al., 2021). For visualizing features such as gene expression, motif activity, group density, we used Nebulosa (Alquicira-Hernandez and Powell, 2021).

### Pseudo-Bulk Profiles

The cells that were annotated as CD14+ monocytes (n=32) and pHSPC (n=32) from the Multiome ATAC-seq data and single-cell ATAC-seq data were used to create ‘pseudo-bulk’ profiles for these cell types using snATACClusteringTools. (https://github.com/UcarLab/snATACClusteringTools). For each sample, we generated bam files for these two cell types. 2 samples were filtered out based on low cell numbers (HDu and jcov124) (only for CD14+), three samples were from the same individual and pooled together (Igtd17, Igtd18, and Igtd19), two samples were filtered out due to clinical reasons (jcov114 from CD14+ only, and jcov49_2 from CD14+ and CD34+), resulting in 28 samples for monocytes and 31 for pHSPC. In pHSPC, peak calling was done using MACS2 (Zhang et al., 2008) with the BAMPE option.

### Differential Accessibility Analyses

For differential accessibility (DA) analyses, good quality bulk and pseudobulk ATAC-seq samples for CD14+ monocytes (n=70) and pHSPC (n=49) were used to generate consensus peak sets using DiffBind (Ross-Innes et al., 2012) by retaining peaks that are detected at least in 2 samples, resulting in 123,477 consensus peaks for monocytes and 126672 peaks for pHSPC. Consensus peaks that are not called in the single-cell object were discarded from downstream analyses leaving us with 108,370 peaks for Monocytes and 117,871 peaks for pHSPC. Consensus peaks were annotated using ChipSeeker (Yu et al., 2015) and the peaks were filtered out based on distance to TSS threshold (<50 KB). Post filtering, we had 96,241 consensus peaks for monocytes and 102,784 peaks for pHSPC. These peaks were then used to identify differentially accessible regions among clinical groups using the cinaR R package. For differential accessibility analyses, we used GLM models as implemented in EdgeR (Robinson et al., 2010; Ucar and Karakaslar, 2021) by conducting pairwise comparisons among the clinical groups. Due to the variability of ages across clinical groups, we used age as a covariate. Finally, to account for known and unknown batches, we used Surrogate Variable Analysis using all significant SVs. Differential peaks at FDR 10% were kept for downstream analyses.

### Functional Enrichment Analyses

The peaks were annotated, including the closest genes using cinaR (2021) and ChlPSeeker (Yu et al., 2015). The TSS regions were defined as −3 KB to 3 KB. In cases of overlap, the following order of genomic annotations is used: Promoter >5′ UTR>3′ UTR>Exon>Intron>Downstream (defined as downstream of gene end)>Intergenic. Functional enrichment for the differential peak sets was conducted using hypergeometric gene set enrichment tests followed by Benjamini-Hochberg FDR adjustment for P-values (cutoff adjusted p=0.1). For functional enrichment, we used different curated gene sets from the CinaRgenesets package (2021). For pHSPC, Gene Set Enrichment Analysis (GSEA) was carried out in addition to the hypergeometric testing since DA peak counts were smaller.

HOMER (Heinz et al., 2010) was used for further functional enrichments for KEGG, GO, REACTOME, etc. (p-value cutoff=0.1). HOMER was also used to identify TF motifs enriched in differential peak sets using default settings both for known and de novo motifs. For motif enrichment analyses, we used the default (whole genome) as a background.

### Time Series/Trend Analysis

Differential peaks from bulk and pseudo-bulk ATAC-seq data were pooled together for the following 4 comparisons: Early vs. Healthy, Late vs. Healthy, and nonCoV vs Healthy for both CD14+ Monocytes and pHSPC. These peaks were then used to detect trends across different clinical groups using tcseq. The read counts and log fold change values associated with the differential peaks for each clinical group were used as input, and the clinical groups served as time points for the purpose of our analysis. A various number of clusters were tested before we chose number of clusters as the ideal number to visualize the different patterns in the clinical groups. The differential peaks were then split into clusters, and the Functional Enrichment was conducted for each of the individual clusters.

### HINT

Footprints are more reliable with deeply sequenced data. To increase the read depth of our data, we pooled Pseudo-bulk ATAC-seq profiles from each clinical group into a single profile for both CD14+ monocytes and pHSPC. Peaks were then called for these pooled profiles using macs2. The HINT (Hmm-based IdeNtification of Transcription factor footprints) framework (Li et al., 2019) was then used to identify active transcription factor binding sites for each clinical group. We also used HINT to find motifs overlapping with the footprints using the JASPAR database for analyzed TF motifs. We then used HINT to generate average ATAC-seq profiles around binding sites of transcription factors of interest. HINT was also used to calculate the differential changes in TF activity between different clinical groups.

The motif regions overlapping with footprints were annotated using ChipSeeker (Yu et al., 2015), and based on those annotations, hypergeometric geneset enrichment was carried out for the footprinting regions using various genetic datasets. The enrichment results were adjusted using the Benjamini-Hochberg FDR adjustment method (FDR=10%).

### Spark Tracks

We also visualized Pseudo-bulk profiles using a package called SparK (Kurtenbach and Harbour, 2019). Firstly, bedgraph files were generated using deepTools (Ramfrez et al., 2014) with the following command ‘bamCoverage-b bamfile.bam-o outputfilename.bdg-bs 1-of bedgraph’. Spark was then used to generate genome browser track Figures using the bedgraph files for selected loci.

### Gene Ontology Analysis

All gene ontology enrichment analyses of differentially expressed genes and genes associated to differentially accessible regions were performed using ClusterProfiler R package (Yu et al., 2012).

### Ex Vivo CD14+ Monocyte Stimulation Assays

PBMCs (n=38) from PBMC-PIE protocol were isolated and monocytes were purified with anti-CD14 magnetic beads (Miltenyi Biotec, 130-050-201) followed by FACS-sorting to remove dead cells. 10-50K cells were plated on 96-well plate in complete RPMI, then stimulated with IFNα (50 ng/ml, PBL assay science) and R848 (1 μM, InvivoGen) for 6 hours. RNA was extracted from cells using the Qiagen RNeasy Mini Kit. Quantity of RNA was measured by Nanodrop, and high-capacity cDNA Reverse Transcription kit was used to generate 20-50 ng cDNA. Gene expression levels were calculated based on relative threshold cycle (Ct) values. This was done using the formula Relative Ct=100×1.8 (HSK−GENE), where HSK is the mean CT of duplicate housekeeping gene runs (we used Ubiquitin), GENE is the mean CT of duplicate runs of the gene of interest, and 100 is arbitrarily chosen as a factor to bring all values above 0.

### Example 3

### Altered Chromatin Accessibility and Durable Epigenetic Memory in Monocytes Following COVID-19

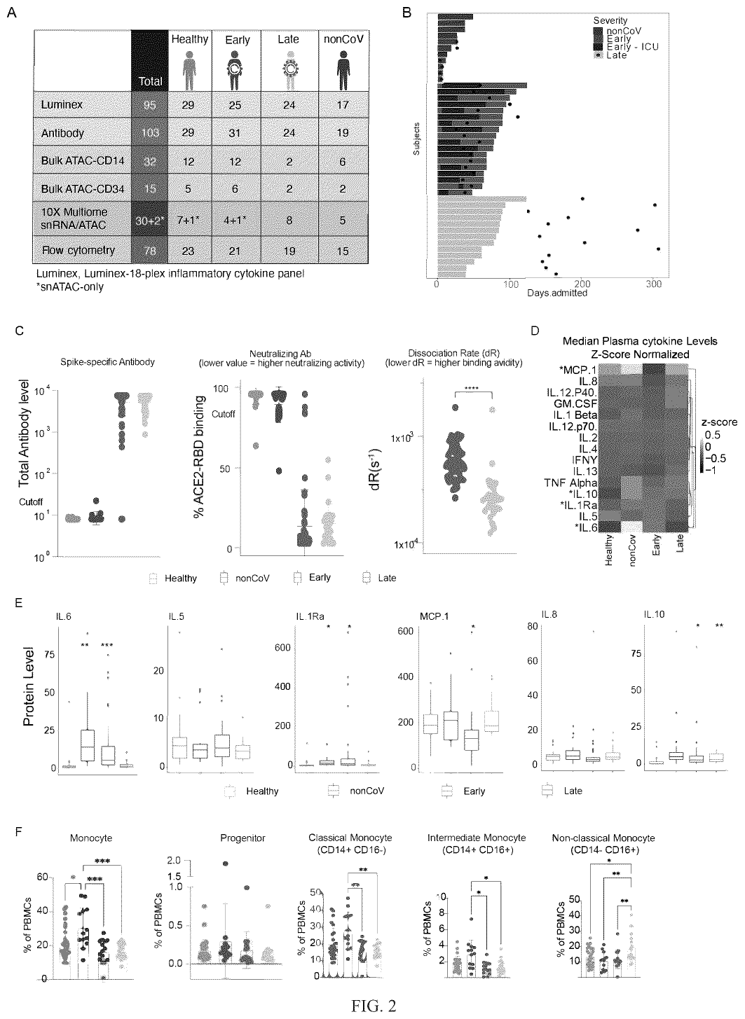

The molecular features of hematopoietic and mature immune cells in convalescent severe COVID-19 study participants (WHO score 6-7, requiring ICU admission) were studied and compared against molecular features of (i) healthy (SARS-CoV-2 naïve) participants and (ii) participants who recovered from non-COVID-19-related critical illness (either infectious- or non-infectious disease) (nonCoV) by profiling peripheral blood mononuclear cell (PBMC) and plasma samples. To study the durability of these molecular features, we collected blood samples from both early convalescent (2-4 months after the onset of the disease, “Early”; plasma analysis, n=31; epigenomics analysis, n=17) and late convalescent participants (4-12 months after the onset, “Late”; plasma analysis, n=24; epigenomics analysis, n=10) (FIG. 2A-B). Study participants were enrolled at Weill Cornell Medicine and New York Presbyterian Hospital during the first wave of infections in New York City (from May 2020 to December 2020) prior to administration of COVID-19 vaccines and were likely infected with 614D and 614G variants of the virus (Koelle et al., 2022). Healthy donors (symptom-free and seronegative) were enrolled by taking into consideration age, sex, and comorbidities to clinically match the COVID-19 clinical group (demographic and clinical information, supplementary data). To comprehensively study cellular and molecular features of immune cells in this cohort, we established a multimodal assay and analysis workflow including combined single nuclei (sn) RNA and Assay for Transposase-Accessible Chromatin (ATAC) sequencing (Chromium Single Cell Multiome ATAC+ Gene Expression) for PBMC, sorted PBMC subset “bulk” ATAC-seq, multiplexed immunoassay-based quantitation of plasma proteins, and immunophenotyping by flow cytometry (FIG. 2A-F).

To determine if mature circulating CD14⁺ monocytes from COVID-19 convalescent individuals have distinct and durable epigenetic signatures, we profiled chromatin accessibility by ATAC-seq across four clinical groups: (i) Healthy, (ii) early convalescence from non-COVID-19 critical illness (“nonCoV”), and (iii) “Early” and (iv) “Late” convalescence following severe COVID-19 (as defined above). From n=57 donors, we profiled monocyte chromatin accessibility landscapes using either the 10× Genomics Multiome snRNA/ATAC-seq (snRNA/ATAC-seq) assay or conventional ATAC-seq. FACS-sorted CD14⁺ monocytes paired with conventional (bulk) ATAC-seq, enabled us to increase our sample size and resulted in a cohort of 57 total donors (Healthy, n=19; nonCoV, n=11; Early, n=17; Late, n=10). Bulk and single-cell “pseudo-bulk” ATAC-seq were analyzed together when appropriate (FIG. 3A, see also Example 2).

After peak calling and quality control, we identified differentially accessible regions (DAR) in monocytes between clinical groups (Early, Late, Healthy, and nonCoV) resulting in 2029 DARs among 96241 consensus peaks (2%). The most significant changes were detected between early convalescent and healthy donors (DAR, n=1917), also confirmed by principal component (PC) analysis of monocyte samples using DAR (FIG. 3B).

Differentially accessible ATAC-seq peaks in monocytes clustered into four major groups based on their accessibility profiles across all clinical groups (FIG. 3C-E). Epigenetic changes between Early and Healthy groups were most significant, where cluster 2 (C2), cluster 3 (C3), and cluster 4 (C4) peaks had increased accessibility and cluster 1 (C1) had decreased accessibility in Early COVID-19 samples (FIGS. 3C and 3E). The nonCoV group exhibited similar epigenetic changes to Early, though with C2 peaks more accessible in Early, and C3 peaks more accessible in nonCoV. C2 and C3 peaks were more accessible in Late monocytes than Healthy, though with reduced average magnitude compared to Early and nonCoV. We therefore refer to C2 and C3 as “persistent” peaks since these disease-related epigenetic changes lasted 4-12 months after the disease onset. In contrast, C4 peak accessibility, while increased in Early, returned toward the Healthy baseline in Late and were therefore referred to as “transient” peaks (FIG. 3E). These transient C4 peaks, most accessible in Early, were enriched for gene ontology (GO) categories including “myeloid leukocyte activation”, “positive regulation of cytokine production”, and “mononuclear cell differentiation” (FIG. 3D) and were annotated to pro-inflammatory and activation molecules (e.g., IL18, CSF1R) (FIG. 3E, boxplots). Consistent with prominent inflammatory programs in early convalescence and independent of cluster-based analysis, PC1 defining peaks were annotated to genes enriched for the GO term “response to cytokine” (adj.pval=0.02) (FIG. 4A, FIG. 3B). Persistent epigenetic features found in monocytes from both Early and Late groups (C2) were enriched for GO categories including “positive regulation of cytokine production”, “monocyte activation”, and “toll-like receptor signaling pathway” (FIG. 3D); were annotated to genes including IL21R, MAPKAPK2, and TNIP2; and were present across individual subjects in nonCoV, Early, and Late groups (FIG. 3E, boxplots). Additional examples of persistent DAR include intronic peaks at CREB1 (CAMP Responsive Element Binding Protein 1) and MMP1 (Matrix Metallopeptidase 1) (FIG. 3F). Individual examples of transient DAR include intronic peaks at CSF1R, the receptor for the critical macroph age differentiation and maintenance factor CSF1, and at IL17RA, a receptor that regulates monocyte differentiation and migration in response to IL-17 (FIG. 3F).

Thus, combined single-cell and bulk chromatin accessibility profiling indicate that circulating monocytes retain altered epigenetic landscapes following recovery from severe COVID-19 with distinct signatures in early and late convalescence. Notably, increased chromatin accessibility at genes encoding cytokines (e.g., CCL3, IL10 and IFNG), adhesion molecules (e.g., CD1D, DOCK5, ADAM9, and ITGAL), and differentiation factors (e.g., KLF13, CREB1, PRKCA, and FOXP1) persisted for one year following acute COVID-19, highlighting either an active maintenance of these programs through ongoing signaling or durable epigenetic memory in monocyte progenitors (FIG. 3E).

### Example 4

### Altered Monocyte Phenotypes and Transcriptional Programs Post-COVID-19

Considering that epigenetic signatures of COVID-19 may be linked to transcriptional changes, we generated combined snRNA/ATAC-seq datasets in PBMC from a subset of our cohort (n=32), which enabled us to study changes in cell subset frequencies and conduct paired analyses of transcriptional and epigenetic programs. We first performed analysis of clinical group-level changes among PBMC. Analysis of snRNA-seq data resulted in 10 clusters reflecting the major PBMC populations, annotated based on the expression of cell type-specific marker genes (FIG. 7D, FIG. 5A, Examples 1-2). Numerous differences in gene expression were detected between clinical groups for each cell type (FIG. 5B). Notably, among all cell types, CD14⁺ monocytes exhibited the largest number of gene expression changes, comparing post-COVID-19 to the Healthy group (1041 DEG for Early, 517 DEGs for Late, FIG. 5B), further supporting the presence of innate immune memory in these short-lived circulating cells. Thus, we focused on myeloid populations and their progenitors for further insights into innate immune memory phenotypes following COVID-19.

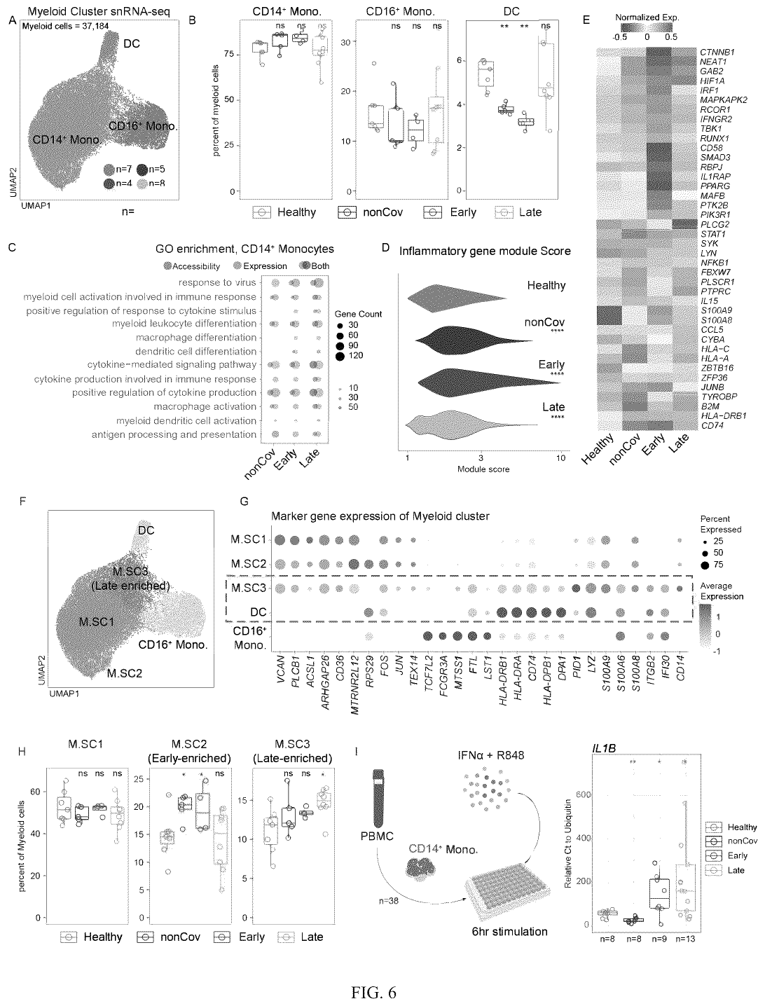

Myeloid cells were initially assessed for frequencies of CD14⁺ monocytes, CD16⁺ monocytes, and dendritic cells (DC) (FIG. 6A-B). At this resolution, we detected a decline in the frequency of circulating DC in Early (post-COVID-19) and nonCoV groups, likely attributable to the inflammation-associated activation and migration of DC into tissues and resultant depletion from circulation (Patel et al., 2017; Zhou et al., 2020), still ongoing 2-4 months following post-acute COVID-19 (FIG. 6B). Circulating DC frequencies (among myeloid cells) returned to the normal range in late convalescence (Late) corresponding to reduced systemic inflammation compared to early convalescence (FIG. 6B).

Next, we focused on the CD14⁺ monocytes and uncovered both differentially expressed genes (DEG) and differential activity in domains of regulatory chromatin (DORC) between disease and the healthy groups (FIG. 6C, FIG. 5D). For DORC analysis, ATAC-seq peaks were linked to their putative target genes in cis, based on the co-variation of chromatin accessibility and gene expression levels across individual cells (Ma et al., 2020) (FIG. 5E-H). DORC represent modules of functionally linked non-coding genome regulatory elements, and DORC-associated genes are highly enriched for those associated with developmental programs and lineage specification and depleted for housekeeping, metabolic, and cell-cycle associated genes. We detected 968 DORC from our data, where at least 15 cis-regulated peaks were associated to each DORC/gene (FIG. 5G-H). Among biologically prominent DORC was one associated with the key monocyte differentiation factor, CEBPA, which was more accessible and more highly expressed in CD14⁺ monocytes compared to hematopoietic stem cell/multipotent progenitor (HSC/MPP) (FIG. 5F).

Next, we performed GO analysis on both DEG and differentially active DORC. We observed enrichment of transcriptional (DEG) and epigenetic (differential DORC) programs relating to “myeloid cell activation involved in immune response” and “positive regulation of cytokine production” in both early convalescent groups (nonCoV and Early COVID-19) (FIG. 6C, FIG. 4B-C). Inflammatory and functional programs in Late monocytes generally overlapped with Early programs and were enriched compared to nonCoV (e.g., “antigen processing and presentation”, “response to virus”, “macrophage differentiation”, “dendritic cell differentiation”, FIG. 6C). Notably, chromatin accessibility signatures (DORC) and active expression programs (RNA) in Late were still prominent (FIG. 6C, FIG. 4B-C), congruent with persistent epigenetic signatures observed in ATAC-seq analysis. Consistent with incomplete resolution of inflammation in early recovery from COVID-19 and non-COVID-19 critical illness, we found that a broadly defined inflammatory gene expression module was transcriptionally upregulated in CD14⁺ monocytes from both groups (FIG. 6D), including genes associated with acute inflammation (e.g., IRF1, STAT1, NFKB1, PPARG, IL1RAP, and MAPKAPK2) (FIG. 6D-E, FIG. 4B). Interestingly, while expression of many of these inflammatory genes returned to baseline in Late COVID-19 convalescence, other pro-inflammatory molecules were persistently upregulated (e.g. pro-alarmins S100A8, S100A9) (FIG. 6D-E, FIG. 4B). We also observed that genes related to antigen presentation including MHC class II genes (e.g., CD74, B2M, HLA-DRB1), were specifically upregulated in Late monocytes (FIG. 6E, FIG. 4B), demonstrating that these cells feature a more differentiated phenotype. Further, we created a browsable web-based interface where DORC, expression, and transcription factor activities can be searched within the single-nuclei myeloid dataset (https://buenrostrolab.shinyapps.io/covid_myeloid/).

Human monocytes have extensive transcriptional heterogeneity (Dutertre et al., 2019). To understand if COVID-19 induced changes in epigenetic and transcriptional programs among CD14⁺ monocytes, we performed further subcluster analysis and identified three CD14⁺ monocytes subclusters (M.SC1, M.SC2, and M.SC3), based on marker gene expression (FIG. 6F-G). We examined individual subject subcluster frequencies across clinical groups (FIG. 6H).

While M.SC1 was equally distributed across groups, M.SC2 was enriched in both early convalescent groups (Early and nonCov) and returned to baseline in Late COVID-19 convalescence and was enriched for epigenomic signatures associated with inflammatory programs such as “positive regulation of leukocyte activation” and “positive regulation of cytokine production” (FIG. 4D). However, no distinct transcriptional programs were found to be enriched for M.SC2, which had a lower level of marker gene expression than M.SC1 but otherwise followed the same pattern. (FIG. 6G, FIG. 4D).

Notably, M.SC3 was unique in that it was enriched more in late convalescence (FIG. 6H). M.SC3 featured typical CD14⁺ monocyte marker genes but was distinguished by increased expression of inflammatory monocyte and DC signature genes including those related to antigen presentation (e.g., CD74, IFI30, HLA genes), migration (e.g., ITGB2), and inflammation (e.g., S100A6, LYZ) (FIG. 6G) (Collin and Bigley, 2018). Thus, complementary and independent analyses (chromatin, expression, single cell, and bulk ATAC-seq) indicated distinct characteristics of late convalescence monocytes, including epigenomic signatures, enrichment of more differentiated M.SC3 monocytes, and differential enrichment of epigenetic and transcriptional signatures associated with antigen presentation, activation, differentiation, and anti-viral responses.

To assess if epigenetic and transcriptional signatures from post-COVID-19 CD14⁺ monocytes are associated with functional differences, we FACS-sorted CD14⁺ monocytes from PBMC (n=38) and stimulated them in vitro with TLR7/8 agonist R848 and IFNa to model an anti-viral response. We assessed CD14⁺ monocyte responses by measuring IL1B transcription, because it has been shown that the IL1B gene can be epigenetically primed in human monocytes for augmented expression to heterologous rechallenge one month later (Arts et al., 2018; Moorlag et al., 2018). We assessed IL1B expression after 6 hours of stimulation, which revealed that CD14⁺ monocytes from both Early and Late convalescent COVID-19 study participants produced significantly more IL1B transcript compared to controls, demonstrating that both epigenetic changes in Early and Late convalescent cells are likely impacting functional responses. Notably, this did not appear to be a common feature of recovery from critical illness as monocytes from post-ICU non-COVID-19 participants produced less IL1B in response to stimulation (FIG. 6I). Associated with this increased responsiveness, we detected increased chromatin accessibility at an upstream enhancer (30 kb) at the IL1B locus in the post-COVID-19 groups (FIG. 4E, indicated).

### Example 5

### Circulating Blood Progenitor Cells Reflect Bone Marrow Progenitor Composition and Phenotypes

Circulating monocytes are short-lived and regularly renewed from hematopoietic progenitors. In the present study, we discovered that the distinguishing characteristics of post-COVID-19 monocytes can derive from altered hematopoiesis or epigenetic phenotypes in progenitor cells that are transferred, through development, to progeny monocytes.

To overcome the impracticalities of obtaining HSPC from bone marrow to study hematopoiesis and epigenetic memory in human disease groups, we developed a platform to enrich and analyze rare circulating CD34⁺ pHSPC (˜0.05% of PBMC). First, we sought to determine if these rare circulating pHSPC captured the diversity, proportions, and epigenetic and transcriptional phenotypes of their bone marrow counterparts. To this end, we enriched CD34⁺ HSPC from both bone marrow mononuclear cells (BMMC) and PBMC from the same donors, spiked them back into total mononuclear cells from these same tissues, and deeply profiled them by combined snRNA/ATAC-seq (FIG. 7A, Examples 1-2). CD34⁺ HSPC of either origin (BMMC and PBMC) co-clustered as a single population in both RNA and ATAC-seq UMAP plots, indicating generally shared transcriptional and epigenomic programs (FIG. 7B). We next annotated HSPC as hematopoietic stem cells and multipotent progenitors (HSC/MPP), lymphoid-primed MPP (LMPP), megakaryocyte-erythroid progenitors (MEP), erythroid progenitors (Ery), granulocyte-monocyte progenitors (GMP), and basophil-eosinophil-mast cell progenitors (BEM), using known marker genes (FIG. 7B, Examples 1-2). We detected all subsets in both BMMC and PBMC derived HSPC, and their transcriptional signatures for lineage-defining marker genes were nearly identical, confirming for the first time in human cells that circulating HSPC capture the diversity and molecular features of HSPC in the bone marrow (FIG. 7B-C).

Thus, while HSPC populations from both tissues have some distinct characteristics, they share extensive transcriptional and epigenetic features, as distinct HSPC subclusters are clearly separated in both snRNA- and snATAC-seq UMAP plots, though tissue of origin comingles (FIG. 7B). Knowledge of this similarity between circulating and BM HSPC led to our broader implementation of in-depth study of circulating HSPC paired with the mature immune cell populations from the same donor, an experimental workflow we termed Peripheral Blood Mononuclear Cell analysis with Progenitor Input Enrichment (PBMC-PIE). This approach is particularly suitable for single-cell profiling of human HPSC in infectious and inflammatory diseases, where HSPC involvement is paramount, little is known, and PBMC are readily attainable.

Thus, we applied the PBMC-PIE workflow to samples from our post-COVID-19 cohort to study phenotypes and epigenetic memory in HSPC. Rare circulating CD34⁺ pHSPC were enriched by sequential antibody-conjugated bead-based enrichment and FACS sorting from PBMCs and then “spiked” into PBMCs from the same donor at an approximate ratio of 1:10, a 200-fold enrichment of their original frequency (FIG. 7D).

### Example 6

### Durable Chromatin Accessibility Signatures in pHSPC Following Severe COVID-19

We profiled PBMC-PIE samples from a subset of our cohort (n=32) using snRNA/ATAC-seq for combined interrogation of gene expression and chromatin accessibility (FIG. 7D-E). Following processing, subclustering, and annotation of the dataset, we then analyzed 28,069 peripheral CD34⁺ pHSPC that manifested all major bone marrow HSPC subsets as defined in our paired BMMC and PBMC HSPC analyses (FIG. 7A-C): HSC/MPP, LMPP, MEP, Ery, GMP, and BEM.

Importantly, projection of these annotations onto snATAC-seq-based pHSPC UMAP visualization also generated distinct pHSPC subclusters (FIG. 7F, right) indicating that these pHSPC subsets feature both distinct expression and epigenetic profiles. Recent single-cell analysis has revealed intriguing heterogeneity among GMP with distinguishing features between BEM and monocyte-neutrophil progenitors (Pellin et al., 2019). In our dataset, these BEM and monocyte-neutrophil progenitor populations were clearly distinguishable, forming distinct subclusters and annotated by characteristic marker genes (FIG. 7C). For monocyte-neutrophil progenitors, these included MPO, VIM, AZU1, and monocyte-neutrophil differentiation receptor, CSF3R, and for BEM, markers included HDC, LMO4, PRG2, and IKZF2 (FIG. 7C).

In addition to the donor-paired analysis of HSPC from BMMC and PBMC above (FIG. 7A-C), we further assessed the suitability of peripheral blood CD34⁺ HSPC from the complete cohort PBMC-PIE dataset as surrogates for bone marrow HSPC. For this further validation, we annotated the peripheral HSPC using (i) bulk ATAC-seq data from FACS-sorted bone marrow HSPC subsets from our previous studies (Buenrostro et al., 2018); and (ii) scRNA-seq data from bone marrow mononuclear cells (BMMC) (Granja et al., 2019). HSPC subcluster annotations transferred from both bone marrow HSPC datasets were projected onto single-cell peripheral HSPC data resulting in two alternative annotations of our pHSPC subsets (FIG. 7G, FIG. 5K-L). The two projections have substantial consensus further supporting the representative diversity of pHSPC and their similarity to BMMC HSPC (FIG. 7G, FIG. 5K-L). Importantly, early multipotent HSC and MPP subsets were prominently represented among peripheral HSPC; CD164, a gene suggested as an early hematopoietic stem cell marker (Pellin et al., 2019), was highly expressed in the HSC/MPP subclusters defined by bulk-ATAC-seq-guided annotation (FIG. 5L-M).

We next sought to broadly define differences in HSPC epigenetic programs across groups, considering that these may provide insight into altered hematopoiesis or epigenetic memory of inflammation that persists in progeny monocytes. To increase our detection power, we supplemented HSPC pseudo-bulk ATAC data derived from the snRNA/ATAC-seq dataset with additional bulk ATAC-seq from FACS-sorted peripheral HSPC, which increased our cohort size to 38 in these analyses.

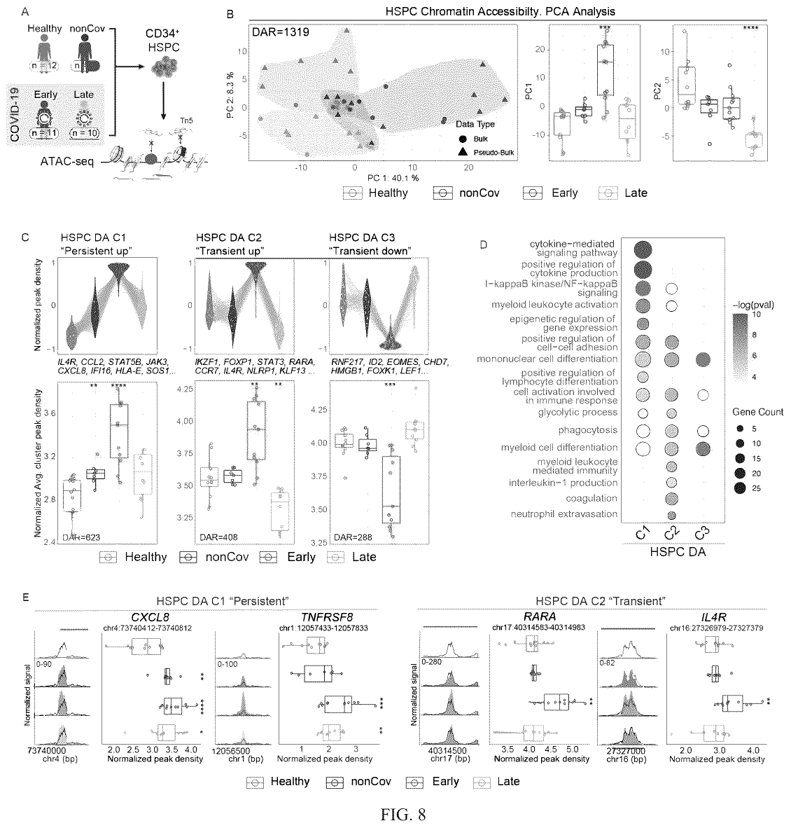

We identified 2271 DARs in pHSPCs when comparing disease groups to healthy controls (FIG. 8A-B). As with monocytes, we found that individual samples from each clinical group clustered together and segregated from other groups in PCA analysis (FIG. 8B). Principal component distributions between clinical groups were significant and again highlighted similarities between both early convalescence groups (PC1, Early and nonCoV) and distinguishing epigenetic features in late convalescence (PC2, Late) (FIG. 8B, box plots). Clustering of DAR revealed three clusters including persistent (C1) and transient (C2) epigenetic programs in pHSPC (FIG. 8C). The persistent DAR (C1) were annotated to genes enriched for GO terms related to differentiation, migration, activation, and cytokine-mediated signaling (e.g., PTPRC, ITGAM, CCL26, IL1RL2, and IFI16) (FIG. 8C-E), indicating establishment of long-lasting epigenetic memory within pHSPC. Transient (C2) DAR were associated with genes related to myeloid differentiation, activation, and cytokine production (e.g., IKZF1, IL4R, RARA, STAT3, and KLF13) (FIG. 8C-D). Notably, while monocytes from both early convalescent groups (nonCoV and Early) shared some overlapping epigenomic features, early convalescent COVID-19 pHSPC participants were distinguished from nonCoV (FIG. 8B-C).

The prominence of the transient program (C2) in Early versus nonCoV groups can reflect the direct effects of increased systemic inflammation on HSPC in severe COVID-19 compared to critical illness in general and have relevance to durable epigenetic effects observed in Late monocytes and pHSPC, as well as functional differences in stimulated monocytes (FIG. 6I). Persistent (C1) and transient (C2) DAR shared programs for migration, activation, and differentiation. Programs specific to persistent (C1) included cytokine-mediated signaling, while the coagulation program was specific to transient (C2) DAR (FIG. 8D).

### Example 7

### Durably Altered Hematopoiesis Following Severe COVID-19

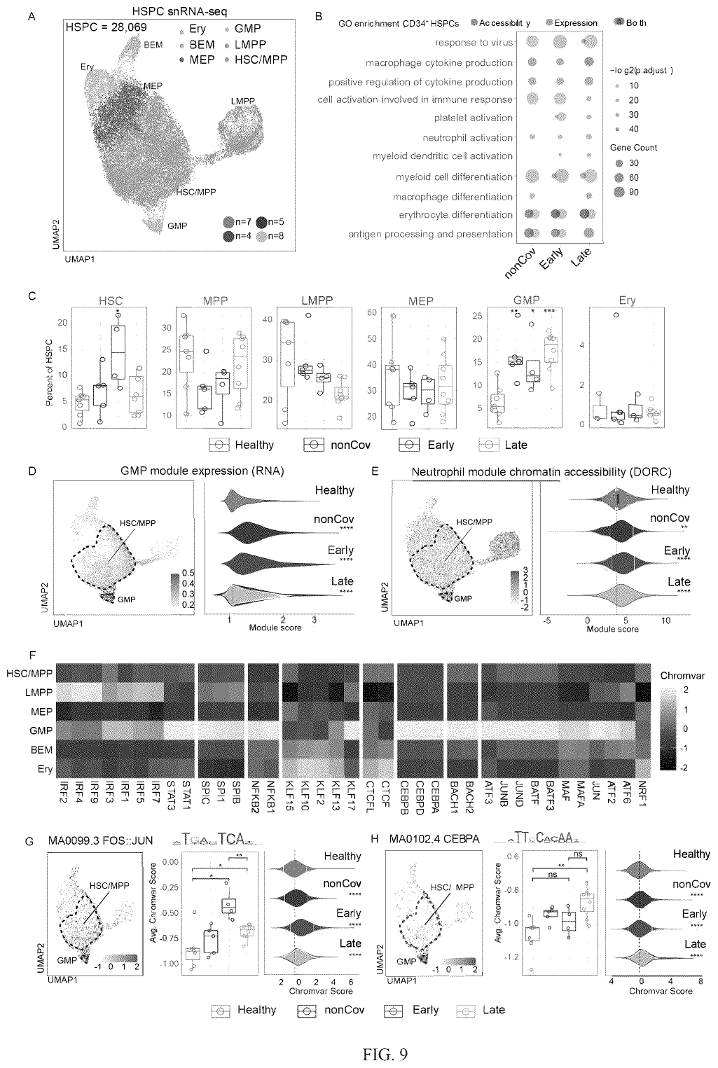

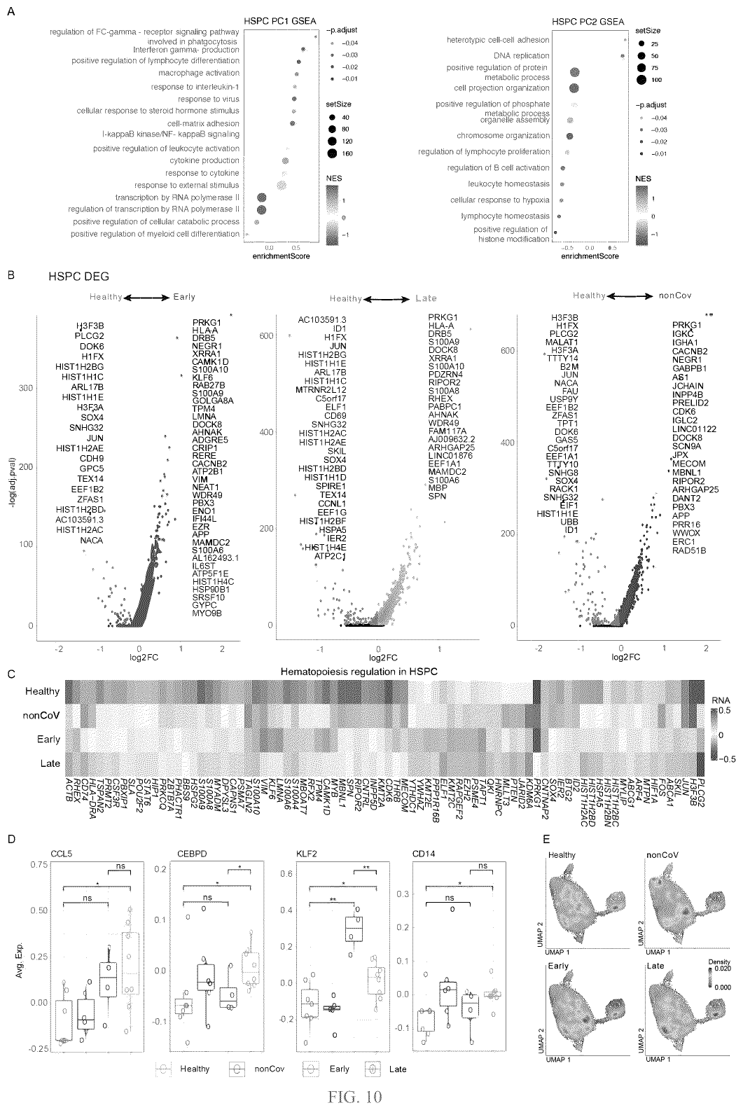

We sought to determine if these epigenomic signatures characterizing post-COVID-19 pHSPC correspond to changes in transcriptional programs or altered progenitor subsets. To this end, we analyzed differences in RNA and chromatin accessibility (DORC) between disease and healthy groups in pHSPC from our combined snRNA/ATAC-seq dataset as we did for myeloid cells. After defining sets of differentially expressed genes and differentially accessible DORC in pHSPC, we observed shared transcriptional and epigenetic programs across all convalescent groups compared to Healthy, including “erythrocyte differentiation” likely reflecting common responses to inflammation and stress (FIG. 9B). In contrast, programs of “myeloid dendritic cell activation” (e.g., RELB, CD2, CAMK4, SLAMF1) and “platelet activation” (e.g., GP1BB, PDGFB, CD40, MYH9) were selectively enriched in the post-COVID-19 state. We also detected GO terms related to cellular activation and cytokine production (e.g., “positive regulation of cytokine production” and “neutrophil activation”) at the epigenetic level only (without prominent expression changes), indicating epigenetic poising of these programs with disease (FIG. 9B).

Analysis of pHSPC DEG in post-COVID-19 groups further supported persistent dysregulation of hematopoiesis and included genes linked to granulopoiesis and myelopoiesis, including CD14, KLF2, CEBPD, and CCL5 (FIG. 10B-D). Supporting evidence of increased granulopoiesis and myelopoiesis in DEG analyses, we found that GMP subcluster frequencies (among pHSPC) were significantly increased in the disease groups (FIG. 9C, FIG. 10E). Notably, GMP frequencies were most elevated in the late convalescent COVID-19 group indicating that this phenotype is both durable and independent of emergency hematopoiesis associated with early recovery from critical illness (i.e., only the Early group revealed increased frequencies in the HSC subcluster) (FIG. 9C, FIG. 10E).

To further study differences in transcriptional regulation of granulopoiesis and myelopoiesis we defined a GMP module consisting of the GMP cluster-defining marker genes and visualized the distribution of this module score among individual cells from each group (FIG. 9D). To determine activity of epigenetic programs of myelo-/granulopoiesis and neutrophil differentiation, we defined genomic regions (DORC) annotated to genes linked to these programs by GO (FIG. 9E). Both expression (FIG. 9D) and chromatin (DORC) modules (FIG. 9E) of GMP and neutrophil differentiation were significantly increased in early convalescent groups. While diminished in magnitude, these signatures persisted into late convalescence indicating a durable epigenetic program driving an active bias in granulopoiesis and myelopoiesis following severe COVID-19 (FIG. 9D-E). Notably, transcriptional and especially epigenetic GMP programs reveal moderate activity in the precursors of GMP—the multipotent and self-renewing HSC/MPP (FIG. 9D-E, UMAP plots)—indicating that COVID-19 or other inflammatory stimuli could durably alter hematopoiesis through persistent epigenetic memory in adult stem cells that biases their differentiation.

We next related the durable epigenetic programs in post-COVID-19 pHSPC with transcription factor (TF) programming of granulopoiesis and myelopoiesis by computing TF motif activity score using chromVAR for each pHSPC subset (Schep et al., 2017). Activity of characteristic GMP TFs, including AP-1 (FOS::JUN) and CEBPA were enriched in the GMP subcluster and also present in HSC/MPP (FIG. 9F-H, FIG. 13). Notably, AP-1 (FOS/JUN) and CEBPA activity in pHSPC was significantly higher in both Early (especially AP-1) and Late (especially CEBPA) post-COVID-19 groups both at the level of individuals (box plots) and cells (chromVAR z-score; violin plots) (FIG. 9G-H). We created a browsable web-based interface where DORC, expression, and transcription factor activities can be searched within the single-nuclei pHSPC dataset (https://buenrostrolab.shinyapps.io/covid_hspc/).

Together, these data revealed that following COVID-19 infection pHSPC epigenomes and transcriptomes are significantly altered and may favor the production of granulocytes and monocytes. Notably, these changes are long-lasting (at least one year in our cohort).

### Example 8

### Post-COVID-19 Epigenetic Signatures and Transcription Factor Programs are Shared Between pHSPC and Monocytes

In addition to skewing progeny cell production, durable post-COVID-19 epigenetic programs (i.e., active or accessible chromatin states) in pHSPC could also be inherited by their mature progeny cells with potential to alter mature cell phenotypes. Chromatin accessibility signatures in pHSPC (FIG. 8B-E) and monocytes (FIG. 3B-F) revealed shared epigenetic and transcriptional programs (e.g., FIG. 6C, FIG. 9B). Unbiased differential motif activity analysis in early and late convalescence (compared with Healthy) revealed the prominent activity of inflammation-responsive TFs in early convalescence, in both pHSPC and monocytes, including the enrichment of AP-1 (FOS/JUN) and IRF family motifs (FIG. 11A, FIG. 13). Consistent with increased GMP frequency phenotypes post-COVID-19 (FIG. 9F-H), pHSPC maintained increased activity of CEBP family members and JUN (FIG. 11A).

While the prominence of AP-1 activity diminished in late convalescence, especially in monocytes, IRF activity remained enriched in both pHSPC and monocytes (FIG. 11A, FIG. 12A). For example, IRF2 showed enriched activity in HSC/MPP and GMP subclusters among pHSPC (both enriched post-COVID-19), and among SC3 monocyte subcluster (enriched for Late group) (FIG. 11B-C, UMAP plots). Further, pHSPC and monocytes from early and late convalescence featured increased activity of IRF2 (distribution of single cell chromVAR scores) (FIG. 11B-C, violin plots). In addition to persistence of IRF family activity, other TFs associated with monocyte differentiation and activation had increased activity in both Late pHSPC and monocytes, including HOX factors, NRF1, and CTCF (FIG. 11A, FIG. 12B-C).

Beyond TF motif accessibility, deeply sequenced ATAC-seq data can be analyzed for TF footprints—occlusion and protection from transposon insertion of the putative TF binding site or k-mer motif—to infer genome-wide chromatin binding activity of specific transcription factors (in a manner independent of chromvar based analyses). Using footprinting analyses, we extended investigation of CTCF and NRF1 activity and discovered increases in chromatin binding (footprint frequencies) of NRF1 and CTCF, shared among pHSPC and monocytes (FIG. 12B-C). Enrichment of CTCF activity in post-COVID-19 pHSPC and monocytes was especially surprising given its established role as a universal TF and architectural chromatin TF. Highly enriched TFs were also present from the KLF, ETS, POU, FOX (foxp1), GATA, HOX, ZNF, zbtb, TBX, RUNX, and SOX families.

Notably, CTCF has been shown to play an important role in monocyte differentiation and function (Koesters et al., 2007; Minderjahn et al., 2022) and its increased activity post-COVID-19 may reflect a chromatin state characteristic of more differentiated monocytes or of monocytes primed to differentiate, with differentiation-associated genes prematurely active or poised with CTCF binding in progenitor populations. Consistent with this, we noted that several genes featured coordinated upregulation in both pHSPC and monocytes post-COVID-19, most prominently and consistently in late convalescence (including antigen presentation related CD74, and neutrophil/GMVP/activation related S100AB/A9) (FIG. 11D).

For more thorough investigation of a shared epigenetic program from pHSPC to monocytes, we generated module scores for the Late-enriched monocyte subcluster 3 (M.SC3) using marker genes for this subcluster and projected this module score onto the pHSPC UMAP to see if it has activity in progenitor cells that may prelude its sustained activity in progeny monocytes (FIG. 11E). We found that the M.SC3 module (e.g., LYZ, CD74, HLA-DRA, IFI30, CD14, S100As) was indeed expressed in pHSPC, and notably, was especially enriched in HSC/MPP and GMP subclusters, which were present at increased frequencies among Early and Late convalescent pHSPC (FIG. 11F, left). Further, we observed an increase in the distribution of this module's activity among pHSPC that persisted into late convalescence (FIG. 11F, right) and corresponded to its increased activity in monocytes up to one year after COVID-19.

### Example 9

### Using Purified or Enriched Peripheral Blood HSPC for Functional Cellular Assays, for Example Colony Forming Assays

Colony forming assays (CFAs) are used to evaluate the function of HSPCs in the diagnosis and prognosis of diseases. CFAs measure the ability of HSPCs to form colonies in a supportive environment and provide information on their potential to produce different types of blood cells. They are commonly used in the diagnosis of hematologic disorders such as leukemia and lymphoma, where an abnormal number or type of colonies can indicate the presence of these tumors. CFAs are also used to evaluate the success of bone marrow transplantation by measuring the number and type of colonies formed by HSPCs in the transplanted sample with a high number of normal colonies indicating successful engraftment and recovery of hematopoietic function.

Herein, we demonstrate that purified HSPC from peripheral blood function in a manner comparable to bone marrow HSPC in CFAs.

Methodologically, we used cryopreserved PBMC to enrich CD34+ cells with human CD34 microbeads (Miltenyi, 130-046-702), then FACS to sort all viable CD34+ cells. Cell counts were tracked. CD34+ cells were collected into a collection tube containing IMDM containing 2% FBS, then centrifuged at 300 g for 5 minutes. To achieve the manufacturer's specified plating concentration, cells were resuspended in IMDM with 2% FBS. For duplicate tests, 300 ul of cell suspension was combined with 3 ml of pre-aliquoted MethoCult™ H4434 Classic (MethoCult™ GF H4434) media. The tube was vortexed for 4 seconds before being let to stand for 5 minutes to allow bubbles to rise to the top. Next, 1.1 ml of mixture was dispensed into a 6-well plate using a 16 gauge blunt-end needle without the introduction of any bubbles. To preserve the moisture, empty gaps or wells on the plates were filled with sterile water or PBS. For 14 days, plates are incubated at 37° C. in 5% CO2. STEMvision™ was used to count and picture colonies.

This establishes purified or enriched peripheral blood HSPC as amenable for use in CFAs or to be used for downstream analysis of any other cell functional characteristics.

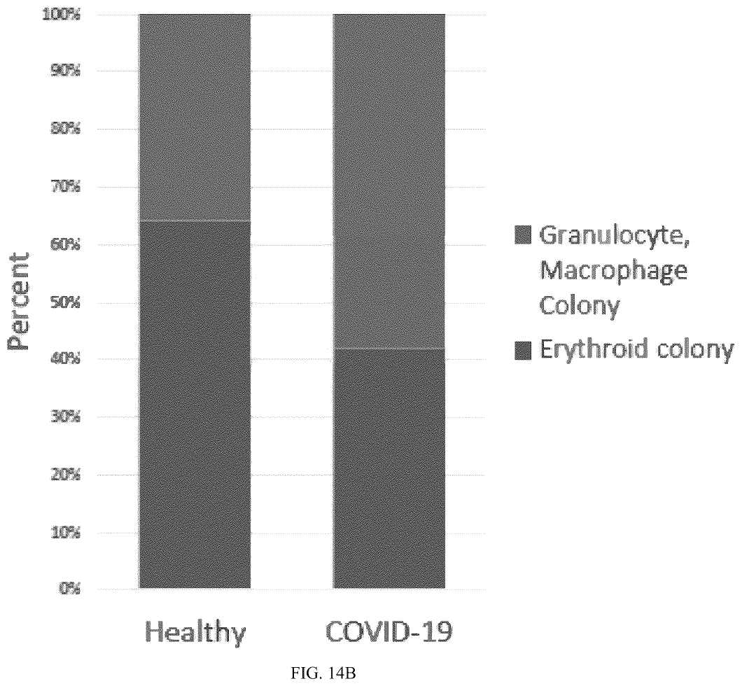

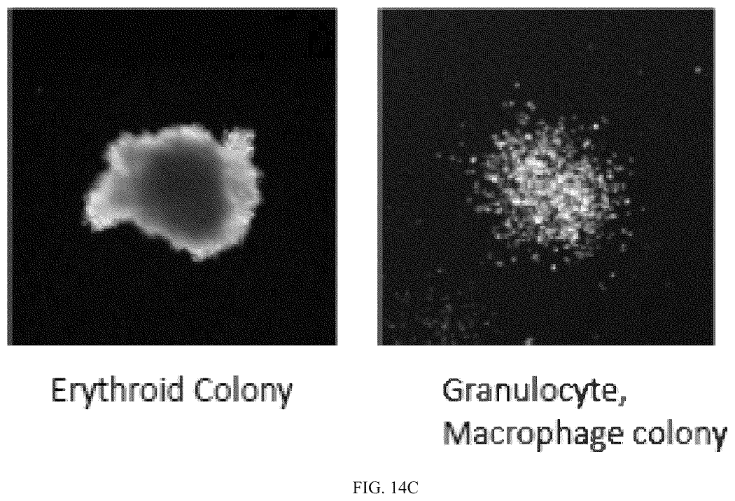

Beyond potential for improvement of standard applications mentioned above (leukemia diagnosis, bone marrow transplant function), we demonstrate that CFAs on purified peripheral HSPC from a healthy donor versus a post-COVID donor reveals distinguishing functional characteristics of the pHSPC and complements a “functional assay”, namely the bias towards granulopoiesis and myelopoiesis that we observe in the molecular data discussed above (FIGS. 14A-C).

### Example 10

### Extracting Genotype and SNP Information from Chromatin Accessibility Data in PBMC-PIE Datasets to Correlate Genotypes and Hematopoietic Phenotypes

Genetic variants, especially “coding variants” that exist in mRNA sequence and non-coding regulatory DNA genetic variants that are enriched (in general and for biological activity) within accessible chromatin regions, can be detected by increasing the sequencing depth of single nuclei ATACseq datasets generated from PBMC-PIE. Thus, extracting, or mining, genotype and single nucleotide polymorphism (SNP) information can be readily extracted from single nuclei ATACseq data from PBMC-PIE can enable correlation between genetic variants and hematopoietic phenotypes in a single assay. Sufficiently large datasets could then drive predictions on genotype-phenotype drivers of disease and therapeutically tractable hematopoietic phenotypes.

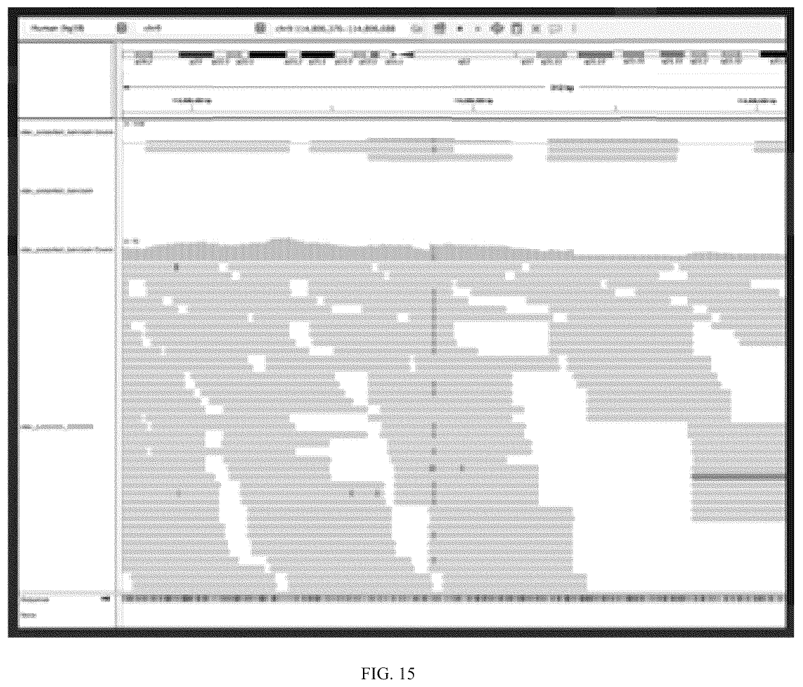

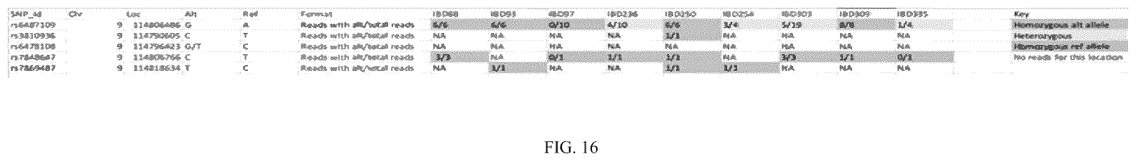

One example of this application is at the TL1A locus, which contains a highly abundant non-coding regulatory element SNP in an upstream enhancer, rs6487109. Mining genetic sequence information from the ATACseq data and visualizing the single base pair frequencies for each individual can determine if an individual is homozygous, heterozygous, or “wild-type” for this variant allele (FIG. 15). Grey, unannotated reads (grey bars) represent the wild-type allele (A) and dark lines within grey bars indicate variant alleles (G). Frequencies of A/G can be used to identify homozygous wild-type A/A, heterozygous A/G, and homozygous mutant (G/G) (FIG. 16).

### Example 11

### Therapeutic Blockade of IL6R and Durable Effects on Hematopoietic Phenotypes, Namely Granulocyte-Monocyte Progenitor Frequencies, as Determined by PBMC-PIE

One of the key biological findings enabled by the presently described PBMC-PIE approach was the durable (at least 12 month) increase in granulocyte-monocyte progenitors (GMP) that persists following acute severe COVID-19. This is one molecular/cellular phenotype that can contribute to long COVID, or long-term clinical sequelae of COVID-19 (and other infections). Understanding the molecular drivers of this durable and potentially pathogenic post-infection program could lead to diagnostic/prognostic markers and the identification of therapeutic targets of such conditions.

Symptoms of long COVID are varied and are under investigation. For example, the Centers for Disease Control (CDC) currently defines long covid symptoms as including: tiredness or fatigue that interferes with daily life; symptoms that get worse after physical or mental effort (also known as “post-exertional malaise”); fever; respiratory and heart symptoms; difficulty breathing or shortness of breath; cough; chest pain; fast-beating or pounding heart (also known as heart palpitations); neurological symptoms; difficulty thinking or concentrating (sometimes referred to as “brain fog”); headache; sleep problems; dizziness when you stand up (lightheadedness); pins-and-needles feelings; change in smell or taste; depression or anxiety; digestive symptoms; diarrhea; stomach pain; other symptoms such as joint or muscle pain, rash, changes in menstrual cycles, etc.

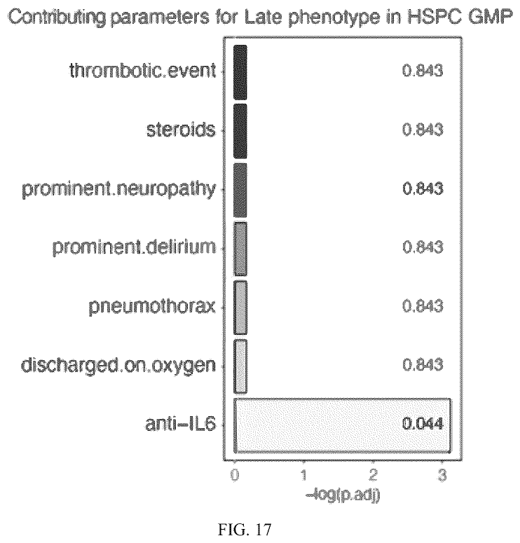

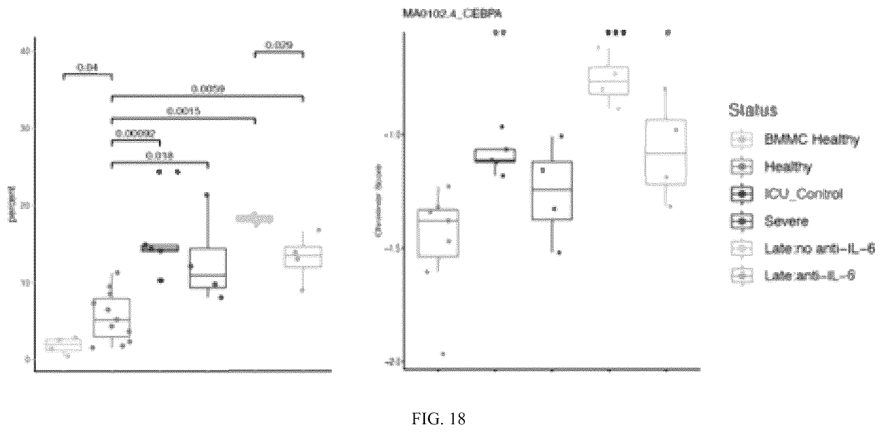

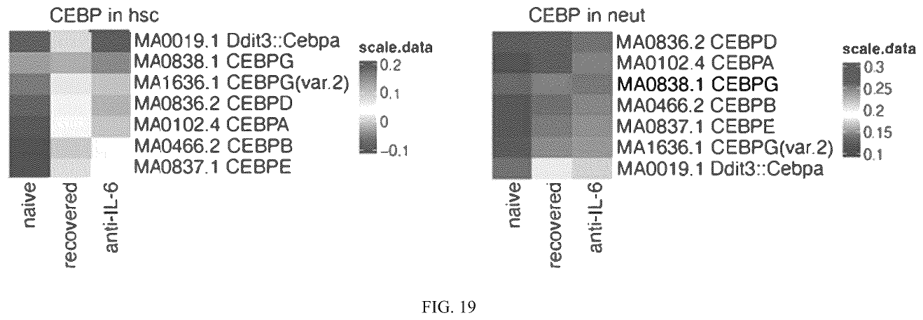

The difference between GMP frequencies in healthy and “late convalescent” post-COVID-19 study participants was statistically significant (p<0.001). However, because there was variance in GMP frequencies in the post-COVID group, the study sought to understand if any parameters (demographic, clinical, treatments, etc.) could explain this variance. In performing multiple-hypothesis testing analysis a single variable emerged that best predicted the variance in GMP frequencies, namely whether individuals received a single dose of IL-6R blocking antibody (Tocilizumab or Sarilumab) during their acute illness, while being treated in the ICU (FIG. 17). Remarkably, this single treatment to block and reduce the biological activity of the inflammatory (and pleiotropic) cytokine IL-6 during acute viral infection was sufficient to reduce the amplitude of the COVID-related increases in GMP precursors up to 12 months later (FIG. 18). Upon further analysis, it was found that other molecular data (as listed above) from PBMC-PIE analysis similarly reflected the durability of IL-6 biological activity; CEBP transcription factors, known to be regulated directly by IL-6, including CEBPB (also known as “nuclear factor IL-6”, or “NF-IL6”), showed reduced activity in individuals who were treated once during acute disease with IL-6R blockade therapy (FIG. 19).

This demonstrates the PBMC-PIE is able to (i) define a disease associated hematopoietic phenotype (e.g. elevated GMP frequencies and underlying epigenetic programs) and (ii) define the therapeutic response among hematopoietic stem and progenitor cells to a treatment condition (e.g. IL-6R blockade), in this case a response that is durable for at least months to one year.

### Example 12

### Modeling Post-COVID-19 Infection in Mice and Validating Phenotypes and Therapies (e.g. IL-6R Blockade)

Human data such as that described above is inherently high-variance due to genetic and environmental factors, as well as clinical comorbidities, treatments, age, sex and other factors. Further, it is difficult to access tissues, such as the lung and the brain in human patients to appreciate correlations between changes in hematopoiesis, circulating immune cells, and tissue pathology. Thus, experiments were conducted to validate the above-described human findings in a mouse model; namely, to explore the function of IL6, during acute coronavirus infection, in durably programming changes in hematopoiesis, including increased frequencies of GMP.

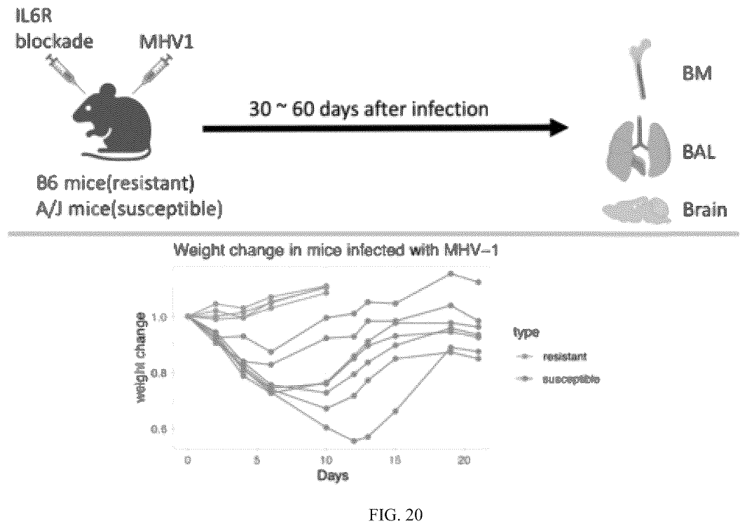

To this end, a mouse model was established of murine hepatitis virus 1 (MHV1)—a well-established mouse model of coronavirus infection and recently used to model SARS-CoV-2 infection. This model is useful because it features disease of varying severity in mice of different genetic backgrounds with B6 mice featuring mild disease (and no weight loss) while A/J mice feature severe disease (similar to our human cohort) and lose considerable weight during acute infection before resolving infection and gaining back weight. A schema of the experimental design and weight loss curves for both strains of mice is shown in FIG. 20. This mouse model was then used to study the effects of IL-6R blockade—administered during acute infection—on durable changes in hematopoiesis, GMP frequencies, immune cell infiltrates into tissues (brain and lung), and pathology.

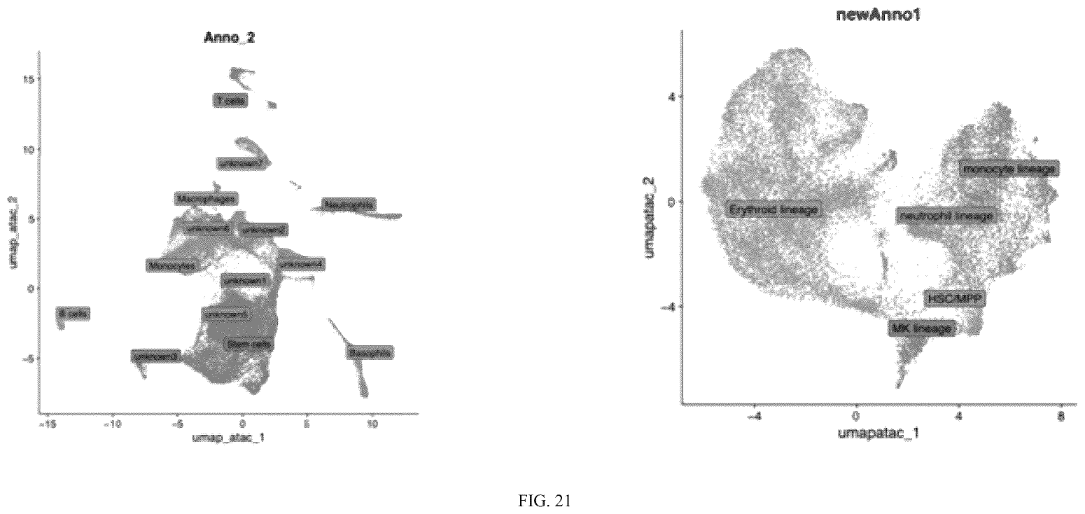

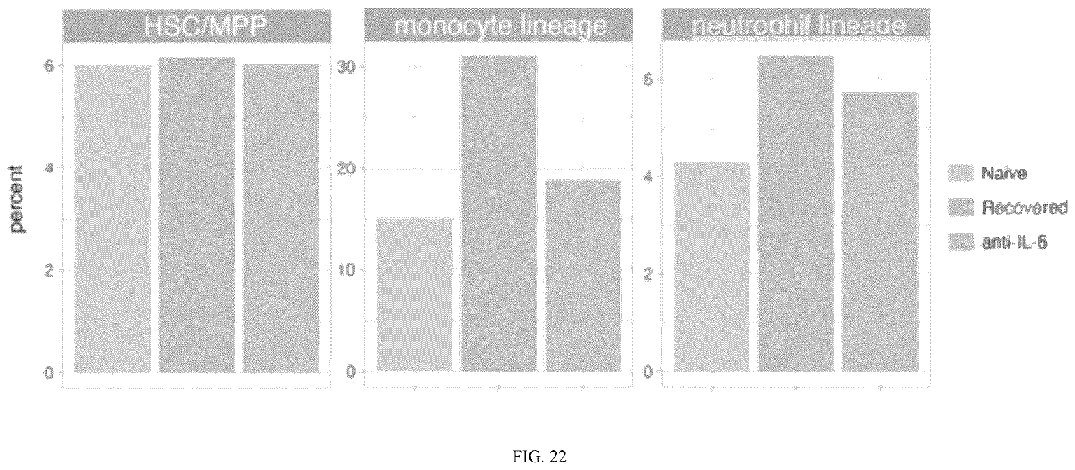

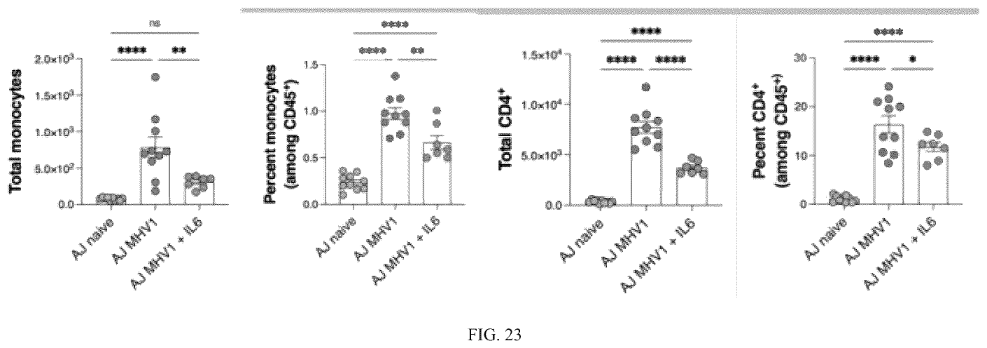

Corresponding with the results in humans, from analyzing single cell data from mouse pHSPC isolated from the bone marrow, it was found that mouse coronavirus infection increases GMP (defined as neutrophil and monocyte progenitors) (FIG. 21, Figure=, and that IL-6R blockade reduced this post-infection increase in GMP. Further, this trend—increased GMP post infection, which is then reduced by IL-6R blockade—was found to correspond to immune cell infiltration into the brain and post-infection pathology (FIG. 23).

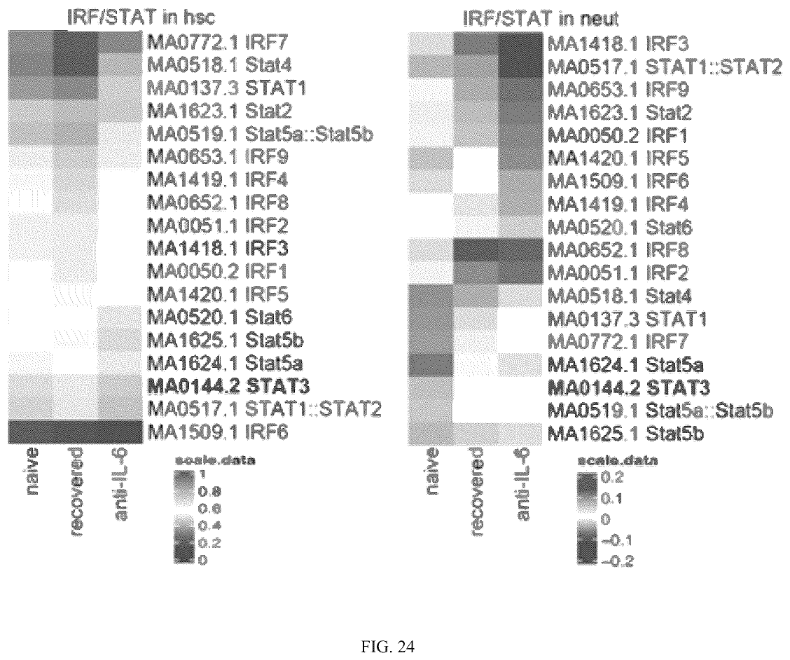

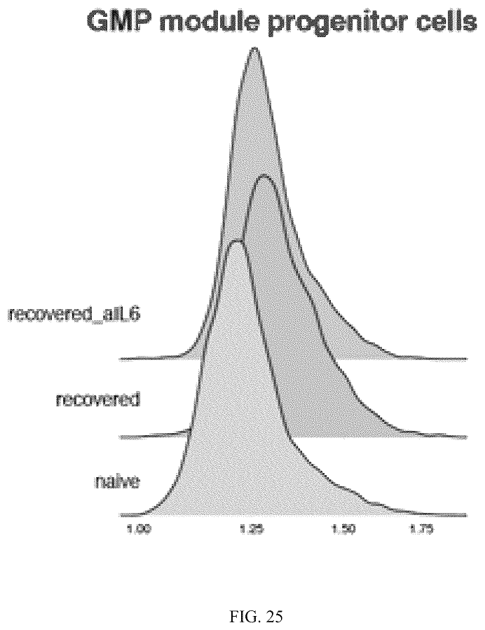

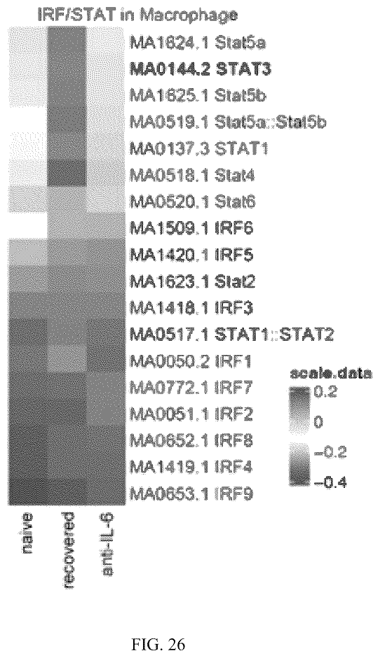

Beyond changes in pHSPC frequencies (e.g. GMP) and tissue infiltration, single cell analysis that matches the technology used in human PBMC-PIE analyses, is able to resolve molecular signatures of post-infection phenotypes. First, increased chromatin binding of transcription factors that control inflammatory programs in post-infection mouse HSC and neutrophil progenitors was found, with this signature being reduced by IL-6R blockade (FIG. 24). This signature correlates with increased (post-infection) and then decreased (with IL-6R blockade) GMP module scores in pHSPC (FIG. 25). Further, IL-6R signals through the transcription factor STAT3, and increased chromatin binding of STAT3 was observed in post-infection mice, an expected change given the activity of IL-6 during infection. Importantly, a decrease in STAT3 activity with IL-6R blockade was also observed in both HSC (FIG. 24) and monocyte/macrophages (FIG. 26). Additionally, the transcription factor CEBPB was found to be increased following infection and reduced by IL-6R blockade (FIG. 25).

Thus, STAT3 and CEBPB inferred transcription factor binding based on ATACseq data represent molecular signatures of a disease state (in this case a post-infection state characterized by persistent brain inflammation) and a response to therapy (IL-6R blockade).

### Example 13

### Methods for Enrichment and Analysis of Plasmacytoid Dendritic Cells (pDCs) in Peripheral Blood

The cellular samples, assays, and analytical techniques used in Example 14 are described below.

Preparation of PBMCs and pDCs

Whole blood or enriched leukocyte buffy coats from healthy donors were obtained from the New York Blood Center (Long Island City, NY) after informed consent of donors who were deemed healthy by the New York Blood Center's criteria. The blood samples were used under a protocol approved by the Institutional Review Board of the Hospital for Special Surgery and the Institutional Biosafety Committee of Weill Cornell Medicine. PBMCs were prepared using Ficoll-Paque density gradient (GE Healthcare) as previously described (Guiducci et al., 2010). For storage of PBMCs in liquid nitrogen, the freezing medium with 10% DMSO+12.5% human serum albumin (HSA) in RPMI 1640 was used. pDCs were isolated from PBMCs by positive selection using BDCA4-MicroBead Kit as previously described (Guiducci et al., 2010).

scRNASeq Sample Preparation, Library Construction and Data Processing

As pDCs are a very rare population in the PBMC, to enrich pDC in the PBMC samples, we first sorted CD3− cells to remove T cells, and then collected CD123+BDCA4+CD14− pDCs and pDC depleted PBMC cells (dT&dpDC). We then merged sorted pDC with the dT&dpDC at a ratio 1:2 before loading to 10× for scRNASeq libraries preparation. Cell viability of all donors are above 85%, 8000 cells from each of eight donors were loaded to the same 10× single cell microfluidic chip to obtain around 5000 recovery cells. Libraries were constructed by following the instructions of 10× company (Chromium Single Cell 3′ Reagent kits v3). The quality of 8 libraries were assessed using Agilent Bioanalyzer 2100 and the concentration were quantified by NEBNext Library Quant Kit (E7630S) and equally merged and sequenced by Novaseq. The sequenced data were analyzed in Cellranger to align reads, generate feature-barcode matrices. A merged dataset from all donors were batch corrected and analyzed using FastMNN (Haghverdi et al., 2018) in a R software based Seurat (Seurat_4.0.3) pipeline (Hao et al., 2021) (https://htmlpreview.github.io/?https://github.com/satijalab/seurat.wrappers/blob/master/docs/fast_mnn. html). Doublets were removed manually by excluding cells with more than two cell type markers. The subclusters of the trimmed dataset is visualized by UMAP plot.

### Differential Gene Expression and Pathway Analysis

We used FindMarkers function (logfc.threhold=0 and min.pct=0.1) of Seurat with Wilcoxon Rank sum test to obtain a list of differentially expressed genes (DEG) in each subset of PBMCs. Genes that had Bonferroni corrected p-value <0.01, and genes with >0.25 log fold changes were considered significantly different. The pathway analysis of the differential expressed genes is analyzed by gsea package (Subramanian et al., 2005) and based on the Reactome pathway database. The principal component analysis (PCA) and top 5 of pathways of each subcluster are analyzed by ReactomeGSA package (Griss et al., 2020).

### Example 14

### Enrichment and Characterization of pDCs in Peripheral Blood

Plasmacytoid dendritic cells (pDCs) are not as rare as HSPC but still less than 1%. To map the heterogeneity of pDCs in human PBMCs, we profiled 10,976 pDCs from the blood of human donors using a pre-enrichment strategy, as conducting single cell analysis of rare cell types is impractical. We identified six subclusters of pDCs, including ISG high clusters. These data revealed the heterogeneity of pDCs in human PBMCs.

We and others have reported that pDCs are key players in the production of IFN-1 response in human PBMCs (Barrat and Su, 2019). However, little is known about the heterogeneity of the pDC subsets in human, in healthy or disease context. As these cells represent less than 1% of total PBMC, the mapping of pDC heterogeneity in human donors has proven to be difficult. For example, in patients with systemic sclerosis, an extensive single cell RNA-seq analysis that sampled 97 SSc patients and 56 controls, both blood and skin, led to the identification of interesting fibroblast subsets but yielded less than 500 pDCs, which does not allow meaningful sub-clustering analysis (Gur et al., 2022). In SLE, the elegant analysis of 276,000 cells from 33 patients with pediatric SLE and 11 matching HDs allowed the mapping of pDC in subclusters (Nehar-Belaid et al., 2020) but with some limitations as only 655 pDCs were analyzed. A second large study of over 1.2M cells from 261 individuals, including 162 SLE patients, yielded less than 4,000 pDCs (Perez et al., 2022). Hence, although the understanding of the transcriptomic landscape at single cell level of pDCs is critical, using un-biased approaches to study rare cell types such as pDCs is creating significant challenges.

Thus, we have developed a sorting-based strategy to pre-enrich pDCs before scRNA-seq analysis, allowing us to sequence and profile more than 10,000 of pDCs using samples from just 8 donors. By using this strategy, we identified six subclusters of pDCs, including ISG high clusters.

A key limitation in conducting RNA-seq at the single cell level with rare cell types, such as pDCs, is the challenge to analyze enough cells to identify meaningful subclusters. Hence, we designed a strategy to enrich our samples with pDCs, as described below.

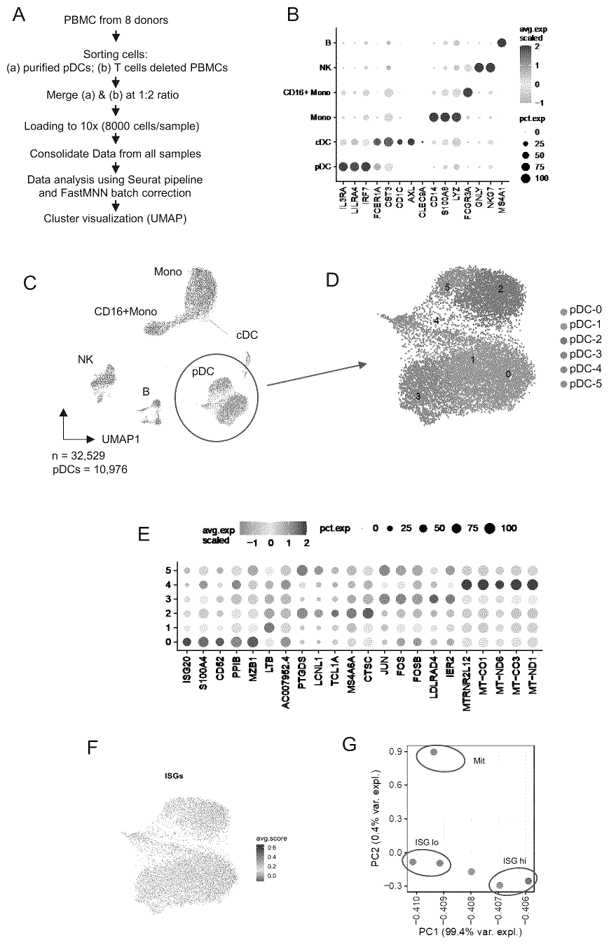

First, CD3+ cells were depleted to remove the T cells which constitute a large cell subset in PBMC. Next, we purified pDCs (CD14⁻ CD123⁺ BDCA4⁺) and mixed the pDCs with the CD3-depleted fraction at a 1:2 ratio, leading to a mix of cells with about 30% pDCs (FIG. 27A). To minimize batch effect, we used previously frozen PBMCs which were all sorted and analyzed on the same day and we obtained 32,529 total cells for scRNA-seq analysis (FIGS. 27B-C). This included 10,976 pDCs as well as monocytes, B cells, NK cells and DC subsets (FIG. 27C).

These data demonstrate that an enrichment strategy allows the analysis of a significant number of cells, even for rare cell types, without the need of large set of donors. In addition to the enrichment, this approach minimized the batch effect, a key issue with scRNA-seq, as all the samples were prepared, sorted and analyzed on the same day.

To determine the heterogeneity in pDCs we defined the pDC population and conducted subcluster analysis. We identified 6 sub-clusters, named pDC-0 to pDC-5, by the UMAP plot in 10,976 pDC cells (FIG. 27D) with the top 5 expressed marker genes of each cluster being shown (FIG. 27E). The mapping of IFN-inducible genes suggested that the pDC-0 and pDC-2 subclusters are associated with high IFN-I response (FIG. 27F). Indeed, pathway analysis by using the gene expression of each cluster projected to principal component analysis (PCA) indicated the association of these 2 clusters with high expression of IFN-1-inducible genes (FIG. 27G). Interestingly, the cells from the pDC-4 cluster appear to contain mostly mitochondrial genes (FIG. 27G); the significance of this subcluster is unclear.

Thus, enriching rare cell types in general, including pHSPC and pDC, within PBMC allows for the study of these rare circulating cell types to conduct scRNA-seq in ways that allows their characterization in and between healthy and/or disease contexts.

### VIII. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein.

It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, now their specific configuration, communication and physical orientation with respect to each other. One skilled in the art should readily appreciate that these components can have various configurations and physical orientations (e.g., wholly separate components, units and subunits of groups of components, different communication regimes between components).

In closing, it is to be understood that the embodiments of the application disclosed herein are illustrative of the principles of the embodiments of the invention. Although specific embodiments and applications of the disclosure have been described in this specification, these embodiments and applications are exemplary only, and many variations are possible. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

Acharya, D., Liu, G., and Gack, M. U. (2020). Dysregulation of type I interferon responses in COVID-19. Nat. Rev. Immunol. 20, 397-398. https://doi.org/10.1038/s41577-020-0346-x.
Agha, R., and Avner, J. R. (2021). Delayed Seasonal RSV Surge Observed During the COVID-19 Pandemic. Pediatrics 148. https://doi.org/10.1542/peds.2021-052089.
Al-Aly, Z., Xie, Y., and Bowe, B. (2021). High-dimensional characterization of post-acute sequelae of COVID-19. Nature 594, 259-264. https://doi.org/10.1038/s41586-021-03553-9.
Alquicira-Hernandez, J., and Powell, J. E. (2021). Nebulosa recovers single cell gene expression signals by kernel density estimation. Bioinformatics https://doi.org/10.1093/bioinformatics/btab003.
Arunachalam, P. S., Wimmers, F., Mok, C. K. P., Perera, R. A. P. M., Scott, M., Hagan, T., Sigal, N., Feng, Y., Bristow, L., Tak-Yin Tsang, O., et al. (2020). Systems biological assessment of immunity to mild versus severe COVID-19 infection in humans. Science 369, 1210-1220. https://doi.org/10.1126/science.abc6261.
Barrat, F. J., and L. Su. 2019. A pathogenic role of plasmacytoid dendritic cells in autoimmunity and chronic viral infection. J Exp Med 216:1974-1985.
Beerman, I., Bhattacharya, D., Zandi, S., Sigvardsson, M., Weissman, I. L., Bryder, D., and Rossi, D. J. (2010). Functionally distinct hematopoietic stem cells modulate hematopoietic lineage potential during aging by a mechanism of clonal expansion. Proc NatlAcad Sci USA 107, 5465-5470. https://doi.org/10.1073/pnas.1000834107.
Bekkering, S., Dominguez-Andrés, J., Joosten, L. A. B., Riksen, N. P., and Netea, M. G. (2021). Trained immunity: reprogramming innate immunity in health and disease. Annu. Rev. Immunol. 39, 667-693. https://doi.org/10.1146/annurev-immunol-102119-073855.
Bell, M. L., Catalfamo, C. J., Farland, L. V., Ernst, K. C., Jacobs, E. T., Klimentidis, Y. C., Jehn, M., and Pogreba-Brown, K. (2021). Post-acute sequelae of COVID-19 in a non-hospitalized cohort: Results from the Arizona CoVHORT. PLoS ONE 16, e0254347. https://doi.org/10.1371/journal.pone.0254347.
Berlin, D. A., Gulick, R. M., and Martinez, F. J. (2020). Severe Covid-19. N. Engl. J. Med. 383, 2451-2460. https://doi.org/10.1056/NEJMcp2009575.
Bogeska, R., Mikecin, A.-M., Kaschutnig, P., Fawaz, M., Buchler-Schäff, M., Le, D., Ganuza, M., Vollmer, A., Paffenholz, S. V., Asada, N., et al. (2022). Inflammatory exposure drives long-lived impairment of hematopoietic stem cell self-renewal activity and accelerated aging. Cell Stem Cell https://doi.org/10.1016/j.stem.2022.06.012.
Bolger, A. M., Lohse, M., and Usadel, B. (2014). Trimmomatic: a flexible trimmer for Illumina sequence data. Bioinformatics 30, 2114-2120. https://doi.org/10.1093/bioinformatics/btu170.
Buenrostro, J. D., Corces, M. R., Lareau, C. A., Wu, B., Schep, A. N., Aryee, M. J., Majeti, R., Chang, H. Y.,
and Greenleaf, W. J. (2018). Integrated Single-Cell Analysis Maps the Continuous Regulatory Landscape of Human Hematopoietic Differentiation. Cell 173, 1535-1548.e16. https://doi.org/10.1016/j.cell.2018.03.074.
Carter, M. J., Fish, M., Jennings, A., Doores, K. J., Wellman, P., Seow, J., Acors, S., Graham, C., Timms,
E., Kenny, J., et al. (2020). Peripheral immunophenotypes in children with multisystem inflammatory syndrome associated with SARS-CoV-2 infection. Nat. Med. 26, 1701-1707. https://doi.org/10.1038/s41591-020-1054-6.
Chen, N., Zhou, M., Dong, X., Qu, J., Gong, F., Han, Y., Qiu, Y., Wang, J., Liu, Y., Wei, Y., et al. (2020). Epidemiological and clinical characteristics of 99 cases of 2019 novel coronavirus pneumonia in Wuhan, China: a descriptive study. Lancet 395, 507-513. https://doi.org/10.1016/S0140-6736(20)30211-7.
Christ, A., Günther, P., Lauterbach, M. A. R., Duewell, P., Biswas, D., Pelka, K., Scholz, C. J., Oosting, M., Haendler, K., Baßler, K., et al. (2018). Western Diet Triggers NLRP3-Dependent Innate Immune Reprogramming. Cell 172, 162-175.e14. https://doi.org/10.1016/j.cell.2017.12.013.
Cirovic, B., de Bree, L. C. J., Groh, L., Blok, B. A., Chan, J., van der Velden, W. J. F. M., Bremmers, M. E. J.,
van Crevel, R., Handler, K., Picelli, S., et al. (2020). BCG vaccination in humans elicits trained immunity via the hematopoietic progenitor compartment. Cell Host Microbe 28, 322-334.e5. https://doi.org/10.1016/j.chom.2020.05.014.
Collin, M., and Bigley, V. (2018). Human dendritic cell subsets: an update. Immunology 154, 3-20. https://doi.org/10.1111/imm.12888.
Consiglio, C. R., Cotugno, N., Sardh, F., Pou, C., Amodio, D., Rodriguez, L., Tan, Z., Zicari, S., Ruggiero, A., Pascucci, G. R., et al. (2020). The Immunology of Multisystem Inflammatory Syndrome in Children with COVID-19. Cell 183, 968-981.e7. https://doi.org/10.1016/j.cell.2020.09.016.
Corces, M. R., Trevino, A. E., Hamilton, E. G., Greenside, P. G., Sinnott-Armstrong, N. A., Vesuna, S., Satpathy, A. T., Rubin, A. J., Montine, K. S., Wu, B., et al. (2017). An improved ATAC-seq protocol reduces background and enables interrogation of frozen tissues. Nat. Methods 14, 959-962. https://doi.org/10.1038/nmeth.4396.
Del Rio, C., Collins, L. F., and Malani, P. (2020). Long-term Health Consequences of COVID-19. JAMA 324, 1723-1724. https://doi.org/10.1001/jama.2020.19719.
Dos Santos, J. C., Barroso de Figueiredo, A. M., Teodoro Silva, M. V., Cirovic, B., de Bree, L. C. J., Damen, M. S. M. A., Moorlag, S. J. C. F. M., Gomes, R. S., Helsen, M. M., Oosting, M., et al. (2019). β-Glucan-Induced Trained Immunity Protects against Leishmania braziliensis Infection: a Crucial Role for IL-32. Cell Rep. 28, 2659-2672.e6. https://doi.org/10.1016/j.celrep.2019.08.004.
Dutertre, C.-A., Becht, E., Irac, S. E., Khalilnezhad, A., Narang, V., Khalilnezhad, S., Ng, P. Y., van den Hoogen, L. L., Leong, J. Y., Lee, B., et al. (2019). Single-Cell Analysis of Human Mononuclear Phagocytes Reveals Subset-Defining Markers and Identifies Circulating Inflammatory Dendritic Cells. Immunity 51, 573-589.e8. https://doi.org/10.1016/j.immuni.2019.08.008.
Fang, C., Mei, J., Tian, H., Liou, Y.-L., Rong, D., Zhang, W., Liao, Q., and Wu, N. (2020). CSF3 Is a Potential Drug Target for the Treatment of COVID-19. Front. Physiol. 11, 605792. https://doi.org/10.3389/fphys.2020.605792.
Friščić, J., Böttcher, M., Reinwald, C., Bruns, H., Wirth, B., Popp, S.-J., Walker, K. I., Ackermann, J. A., Chen, X., Turner, J., et al. (2021). The complement system drives local inflammatory tissue priming by metabolic reprogramming of synovial fibroblasts. Immunity 54, 1002-1021.e10. https://doi.org/10.1016/j.immuni.2021.03.003.
Granja, J. M., Klemm, S., McGinnis, L. M., Kathiria, A. S., Mezger, A., Corces, M. R., Parks, B., Gars, E., Liedtke, M., Zheng, G. X. Y., et al. (2019). Single-cell multiomic analysis identifies regulatory programs in mixed-phenotype acute leukemia. Nat. Biotechnol. 37, 1458-1465. https://doi.org/10.1038/s41587-019-0332-7.
Granja, J. M., Corces, M. R., Pierce, S. E., Bagdatli, S. T., Choudhry, H., Chang, H. Y., and Greenleaf, W. J. (2021). ArchR is a scalable software package for integrative single-cell chromatin accessibility analysis. Nat. Genet. 53, 403-411. https://doi.org/10.1038/s41588-021-00790-6.
Griss, J., G. Viteri, K. Sidiropoulos, V. Nguyen, A. Fabregat, and H. Hermjakob. 2020. ReactomeGSA-Efficient Multi-Omics Comparative Pathway Analysis. Mol Cell Proteomics 19:2115-2125.
Gruber, C. N., Patel, R. S., Trachtman, R., Lepow, L., Amanat, F., Krammer, F., Wilson, K. M., Onel, K., Geanon, D., Tuballes, K., et al. (2020). Mapping Systemic Inflammation and Antibody Responses in Multisystem Inflammatory Syndrome in Children (MIS-C). Cell 183, 982-995.e14. https://doi.org/10.1016/j.cell.2020.09.034.
Guan, W.-J., Ni, Z.-Y., Hu, Y., Liang, W.-H., Ou, C.-Q., He, J.-X., Liu, L., Shan, H., Lei, C.-L., Hui, D. S. C., et al. (2020). Clinical characteristics of coronavirus disease 2019 in china. N. Engl. J. Med. 382, 1708-1720. https://doi.org/10.1056/NEJMoa2002032.
Guiducci, C., M. Gong, Z. Xu, M. Gill, D. Chaussabel, T. Meeker, J. H. Chan, T. Wright, M. Punaro, S. Bolland, V. Soumelis, J. Banchereau, R. L. Coffman, V. Pascual, and F. J. Barrat. 2010. TLR recognition of self nucleic acids hampers glucocorticoid activity in lupus. Nature 465:937-941.
Gur, C., S. Y. Wang, F. Sheban, M. Zada, B. Li, F. Kharouf, H. Peleg, S. Aamar, A. Yalin, D. Kirschenbaum, Y. Braun-Moscovici, D. A. Jaitin, T. Meir-Salame, E. Hagai, B. K. Kragesteen, B. Avni, S. Grisariu, C. Bornstein, S. Shlomi-Loubaton, E. David, R. Shreberk-Hassidim, V. Molho-Pessach, D. Amar, T. Tzur, R. Kuint, M. Gross, O. Barboy, A. Moshe, L. Fellus-Alyagor, D. Hirsch, Y. Addadi, S. Erenfeld, M. Biton, T. Tzemach, A. Elazary, Y. Naparstek, R. Tzemach, A. Weiner, A. Giladi, A. Balbir-Gurman, and I. Amit. 2022. LGR5 expressing skin fibroblasts define a major cellular hub perturbed in scleroderma. Cell 185:1373-1388 e1320.
Hadjadj, J., Yatim, N., Barnabei, L., Corneau, A., Boussier, J., Smith, N., Péré, H., Charbit, B., Bondet, V., Chenevier-Gobeaux, C., et al. (2020). Impaired type I interferon activity and inflammatory responses in severe COVID-19 patients. Science 369, 718-724. https://doi.org/10.1126/science.abc6027.
Haghverdi, L., A. T. L. Lun, M. D. Morgan, and J. C. Marioni. 2018. Batch effects in single-cell RNA-sequencing data are corrected by matching mutual nearest neighbors. Nat Biotechnol 36:421-427.
Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell 184, 3573-3587.e29. https://doi.org/10.1016/j.cell.2021.04.048.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol. Cell 38, 576-589. https://doi.org/10.1016/j.molcel.2010.05.004.
Holter, J. C., Pischke, S. E., de Boer, E., Lind, A., Jenum, S., Holten, A. R., Tonby, K., Barratt-Due, A., Sokolova, M., Schjalm, C., et al. (2020). Systemic complement activation is associated with respiratory failure in COVID-19 hospitalized patients. Proc Natl Acad Sci USA 117, 25018-25025. https://doi.org/10.1073/pnas.2010540117.
Huang, C., Wang, Y., Li, X., Ren, L., Zhao, J., Hu, Y., Zhang, L., Fan, G., Xu, J., Gu, X., et al. (2020). Clinical features of patients infected with 2019 novel coronavirus in Wuhan, China. Lancet 395, 497-506. https://doi.org/10.1016/S0140-6736(20)30183-5.
Huang, L., Yao, Q., Gu, X., Wang, Q., Ren, L., Wang, Y., Hu, P., Guo, L., Liu, M., Xu, J., et al. (2021). 1-year outcomes in hospital survivors with COVID-19: a longitudinal cohort study. Lancet 398, 747-758. https://doi.org/10.1016/S0140-6736(21)01755-4.
Israelow, B., Song, E., Mao, T., Lu, P., Meir, A., Liu, F., Alfajaro, M. M., Wei, J., Dong, H., Homer, R. J., et al. (2020). Mouse model of SARS-CoV-2 reveals inflammatory role of type I interferon signaling. J. Exp. Med. 217. https://doi.org/10.1084/jem.20201241.
Jaffri, A., and Jaffri, U. A. (2020). Post-Intensive care syndrome and COVID-19: crisis after a crisis?Heart Lung 49, 883-884. https://doi.org/10.1016/j.hrtlng.2020.06.006.
Kaufmann, E., Sanz, J., Dunn, J. L., Khan, N., Mendonga, L. E., Pacis, A., Tzelepis, F., Pernet, E., Dumaine, A., Grenier, J.-C., et al. (2018). BCG Educates Hematopoietic Stem Cells to Generate Protective Innate Immunity against Tuberculosis. Cell 172, 176-190.e19. https://doi.org/10.1016/j.cell.2017.12.031.
Kleinnijenhuis, J., Quintin, J., Preijers, F., Joosten, L. A. B., Ifrim, D. C., Saeed, S., Jacobs, C., van Loenhout, J., de Jong, D., Stunnenberg, H. G., et al. (2012). Bacille Calmette-Guerin induces NOD2-dependent nonspecific protection from reinfection via epigenetic reprogramming of monocytes. Proc Natl Acad Sci USA 109, 17537-17542. https://doi.org/10.1073/pnas.1202870109.
Kleinnijenhuis, J., Quintin, J., Preijers, F., Joosten, L. A. B., Jacobs, C., Xavier, R. J., van der Meer, J. W. M., van Crevel, R., and Netea, M. G. (2014). BCG-induced trained immunity in NK cells: Role for non-specific protection to infection. Clin. Immunol. 155, 213-219. https://doi.org/10.1016/j.clim.2014.10.005.
Koelle, K., Martin, M. A., Antia, R., Lopman, B., and Dean, N. E. (2022). The changing epidemiology of SARS-CoV-2. Science 375, 1116-1121. https://doi.org/10.1126/science.abm4915.
Koesters, C., Unger, B., Bilic, I., Schmidt, U., Bluml, S., Lichtenberger, B., Schreiber, M., Stockl, J., and Ellmeier, W. (2007). Regulation of dendritic cell differentiation and subset distribution by the zinc finger protein CTCF. Immunol. Lett. 109, 165-174. https://doi.org/10.1016/j.imlet.2007.02.006.
Kong, L., Moorlag, S. J. C. F. M., Lefkovith, A., Li, B., Matzaraki, V., van Emst, L., Kang, H. A., Latorre, I., Jaeger, M., Joosten, L. A. B., et al. (2021). Single-cell transcriptomic profiles reveal changes associated with BCG-induced trained immunity and protective effects in circulating monocytes. Cell Rep. 37, 110028. https://doi.org/10.1016/j.celrep.2021.110028.
Korsunsky, I., Millard, N., Fan, J., Slowikowski, K., Zhang, F., Wei, K., Baglaenko, Y., Brenner, M., Loh, P.-R., and Raychaudhuri, S. (2019). Fast, sensitive and accurate integration of single-cell data with Harmony. Nat. Methods 16, 1289-1296. https://doi.org/10.1038/s41592-019-0619-0.
Kotfis, K., Williams Roberson, S., Wilson, J. E., Dabrowski, W., Pun, B. T., and Ely, E. W. (2020). COVID-19: ICU delirium management during SARS-CoV-2 pandemic. Crit. Care 24, 176. https://doi.org/10.1186/si3054-020-02882-x.
Kurtenbach, S., and Harbour, J. W. (2019). SparK: A Publication-quality NGS Visualization Tool. BioRxiv https://doi.org/10.1101/845529.
Larsen, S. B., Cowley, C. J., Sajjath, S. M., Barrows, D., Yang, Y., Carroll, T. S., and Fuchs, E. (2021). Establishment, maintenance, and recall of inflammatory memory. Cell Stem Cell 28, 1758-1774.e8. https://doi.org/10.1016/j.stem.2021.07.001.
de Laval, B., Maurizio, J., Kandalla, P. K., Brisou, G., Simonnet, L., Huber, C., Gimenez, G., Matcovitch-Natan, O., Reinhardt, S., David, E., et al. (2020). C/EBPβ-Dependent Epigenetic Memory Induces Trained Immunity in Hematopoietic Stem Cells. Cell Stem Cell 26, 657-674.e8. https://doi.org/10.1016/j.stem.2020.01.017.
Liao, Y., Smyth, G. K., and Shi, W. (2014). featureCounts: an efficient general purpose program for assigning sequence reads to genomic features. Bioinformatics 30, 923-930. https://doi.org/10.1093/bioinformatics/btt656.
Li, H. (2013). Aligning sequence reads, clone sequences and assembly contigs with BWA-MEM.
Li, H., Handsaker, B., Wysoker, A., Fennell, T., Ruan, J., Homer, N., Marth, G., Abecasis, G., Durbin, R., and 1000 Genome Project Data Processing Subgroup (2009). The Sequence Alignment/Map format and SAMtools. Bioinformatics 25, 2078-2079. https://doi.org/10.1093/bioinformatics/btp352.
Li, Z., Schulz, M. H., Look, T., Begemann, M., Zenke, M., and Costa, I. G. (2019). Identification of transcription factor binding sites using ATAC-seq. Genome Biol. 20, 45. https://doi.org/10.1186/s13059-019-1642-2.
Lucas, C., Wong, P., Klein, J., Castro, T. B. R., Silva, J., Sundaram, M., Ellingson, M. K., Mao, T., Oh, J. E., Israelow, B., et al. (2020). Longitudinal analyses reveal immunological misfiring in severe COVID-19. Nature 584, 463-469. https://doi.org/10.1038/s41586-020-2588-y.
Ma, S., Zhang, B., LaFave, L. M., Earl, A. S., Chiang, Z., Hu, Y., Ding, J., Brack, A., Kartha, V. K., Tay, T., et al. (2020). Chromatin Potential Identified by Shared Single-Cell Profiling of RNA and Chromatin. Cell 183, 1103-1116.e20. https://doi.org/10.1016/j.cell.2020.09.056.
Mehandru, S., and Merad, M. (2022). Pathological sequelae of long-haul COVID. Nat. Immunol. 23, 194-202. https://doi.org/10.1038/s41590-021-01104-y.
Minderjahn, J., Fischer, A., Maier, K., Mendes, K., Nuetzel, M., Raithel, J., Stanewsky, H., Ackermann, U., Månsson, R., Gebhard, C., et al. (2022). Postmitotic differentiation of human monocytes requires cohesin-structured chromatin. Nat. Commun. 13, 4301. https://doi.org/10.1038/s41467-022-31892-2.
Mitroulis, I., Ruppova, K., Wang, B., Chen, L.-S., Grzybek, M., Grinenko, T., Eugster, A., Troullinaki, M., Palladini, A., Kourtzelis, I., et al. (2018). Modulation of myelopoiesis progenitors is an integral component of trained immunity. Cell 172, 147-161.e12. https://doi.org/10.1016/j.cell.2017.11.034.
Morris, S. B., Schwartz, N. G., Patel, P., Abbo, L., Beauchamps, L., Balan, S., Lee, E. H., Paneth-Pollak, R., Geevarughese, A., Lash, M. K., et al. (2020). Case Series of Multisystem Inflammatory Syndrome in Adults Associated with SARS-CoV-2 Infection—United Kingdom and United States, March-August 2020. MMWR Morb Mortal Wkly Rep 69, 1450-1456. https://doi.org/10.15585/mmwr.mm6940e1.
Murphy, A. J., and Tall, A. R. (2016). Disordered haematopoiesis and athero-thrombosis. Eur. Heart J. 37, 1113-1121. https://doi.org/10.1093/eurheartj/ehv718.
Naik, S., Larsen, S. B., Gomez, N.C., Alaverdyan, K., Sendoel, A., Yuan, S., Polak, L., Kulukian, A., Chai, S., and Fuchs, E. (2017). Inflammatory memory sensitizes skin epithelial stem cells to tissue damage. Nature 550, 475-480. https://doi.org/10.1038/nature24271.
Nehar-Belaid, D., S. Hong, R. Marches, G. Chen, M. Bolisetty, J. Baisch, L. Walters, M. Punaro, R. J. Rossi, C. H. Chung, R. P. Huynh, P. Singh, W. F. Flynn, J. A. Tabanor-Gayle, N. Kuchipudi, A. Mejias, M. A. Collet, A. L. Lucido, K. Palucka, P. Robson, S. Lakshminarayanan, O. Ramilo, T. Wright, V. Pascual, and J. F. Banchereau. 2020. Mapping systemic lupus erythematosus heterogeneity at the single-cell level. Nat Immunol 21:1094-1106.
Netea, M. G., Quintin, J., and van der Meer, J. W. M. (2011). Trained immunity: a memory for innate host defense. Cell Host Microbe 9, 355-361. https://doi.org/10.1016/j.chom.2011.04.006.
Netea, M. G., Dominguez-Andres, J., Barreiro, L. B., Chavakis, T., Divangahi, M., Fuchs, E., Joosten, L. A. B., van der Meer, J. W. M., Mhlanga, M. M., Mulder, W. J. M., et al. (2020). Defining trained immunity and its role in health and disease. Nat. Rev. Immunol. 20, 375-388. https://doi.org/10.1038/s41577-020-0285-6.
Niec, R. E., Rudensky, A. Y., and Fuchs, E. (2021). Inflammatory adaptation in barrier tissues. Cell 184, 3361-3375. https://doi.org/10.1016/j.cell.2021.05.036.
Ordovas-Montanes, J., Beyaz, S., Rakoff-Nahoum, S., and Shalek, A. K. (2020). Distribution and storage of inflammatory memory in barrier tissues. Nat. Rev. Immunol. 20, 308-320. https://doi.org/10.1038/s41577-019-0263-z.
Patel, A. A., Zhang, Y., Fullerton, J. N., Boelen, L., Rongvaux, A., Maini, A. A., Bigley, V., Flavell, R. A., Gilroy, D. W., Asquith, B., et al. (2017). The fate and lifespan of human monocyte subsets in steady state and systemic inflammation. J. Exp. Med. 214, 1913-1923. https://doi.org/10.1084/jem.20170355.
Pellin, D., Loperfido, M., Baricordi, C., Wolock, S. L., Montepeloso, A., Weinberg, O. K., Biffi, A., Klein, A. M., and Biasco, L. (2019). A comprehensive single cell transcriptional landscape of human hematopoietic progenitors. Nat. Commun. 10, 2395. https://doi.org/10.1038/s41467-019-10291-0.
Perez, R. K., M. G. Gordon, M. Subramaniam, M. C. Kim, G. C. Hartoularos, S. Targ, Y. Sun, A. Ogorodnikov, R. Bueno, A. Lu, M. Thompson, N. Rappoport, A. Dahl, C. M. Lanata, M. Matloubian, L. Maliskova, S. S. Kwek, T. Li, M. Slyper, J. Waldman, D. Dionne, O. Rozenblatt-Rosen, L. Fong, M. Dall'Era, B. Balliu, A. Regev, J. Yazdany, L. A. Criswell, N. Zaitlen, and C. J. Ye. 2022. Single-cell RNA-seq reveals cell type-specific molecular and genetic associations to lupus. Science 376:eabf1970.
Phetsouphanh, C., Darley, D. R., Wilson, D. B., Howe, A., Munier, C. M. L., Patel, S. K., Juno, J. A., Burrell, L. M., Kent, S. J., Dore, G. J., et al. (2022). Immunological dysfunction persists for 8 months following initial mild-to-moderate SARS-CoV-2 infection. Nat. Immunol. 23, 210-216. https://doi.org/10.1038/s41590-021-01113-x.
Proal, A. D., and VanElzakker, M. B. (2021). Long COVID or Post-acute Sequelae of COVID-19 (PASC): An Overview of Biological Factors That May Contribute to Persistent Symptoms. Front. Microbiol. 12, 698169. https://doi.org/10.3389/fmicb.2021.698169.
Quintin, J., Saeed, S., Martens, J. H. A., Giamarellos-Bourboulis, E. J., Ifrim, D. C., Logie, C., Jacobs, L., Jansen, T., Kullberg, B.-J., Wijmenga, C., et al. (2012). Candida albicans infection affords protection against reinfection via functional reprogramming of monocytes. Cell Host Microbe 12, 223-232. https://doi.org/10.1016/j.chom.2012.06.006.
Racine-Brzostek, S. E., Yee, J. K., Sukhu, A., Qiu, Y., Rand, S., Barone, P. D., Hao, Y., Yang, H. S., Meng, Q. H., Apple, F. S., et al. (2021). Rapid, robust, and sustainable antibody responses to mRNA COVID-19 vaccine in convalescent COVID-19 individuals. JCI Insight.
Ramakrishnan, R. K., Kashour, T., Hamid, Q., Halwani, R., and Tleyjeh, I. M. (2021). Unraveling the Mystery Surrounding Post-Acute Sequelae of COVID-19. Front. Immunol. 12, 686029. https://doi.org/10.3389/fimmu.2021.686029.
Ramírez, F., Dündar, F., Diehl, S., Grüning, B. A., and Manke, T. (2014). deepTools: a flexible platform for exploring deep-sequencing data. Nucleic Acids Res. 42, W187-91. https://doi.org/10.1093/nar/gku365.
Ren, G., Cui, K., Zhang, Z., and Zhao, K. (2015). Division of labor between IRF1 and IRF2 in regulating different stages of transcriptional activation in cellular antiviral activities. Cell Biosci. 5, 17. https://doi.org/10.1186/s13578-015-0007-0.
Robinson, J. T., Thorvaldsdóttir, H., Winckler, W., Guttman, M., Lander, E. S., Getz, G., and Mesirov, J. P. (2011). Integrative genomics viewer. Nat. Biotechnol. 29, 24-26. https://doi.org/10.1038/nbt.1754.
Robinson, M. D., McCarthy, D. J., and Smyth, G. K. (2010). edgeR: a Bioconductor package for differential expression analysis of digital gene expression data. Bioinformatics 26, 139-140. https://doi.org/10.1093/bioinformatics/btp616.
Rohde, D., Vandoorne, K., Lee, I.-H., Grune, J., Zhang, S., McAlpine, C. S., Schloss, M. J., Nayar, R., Courties, G., Frodermann, V., et al. (2022). Bone marrow endothelial dysfunction promotes myeloid cell expansion in cardiovascular disease. Nat. Cardiovasc. Res. 1, 28-44. https://doi.org/10.1038/s44161-021-00002-8.
Ross-Innes, C. S., Stark, R., Teschendorff, A. E., Holmes, K. A., Ali, H. R., Dunning, M. J., Brown, G. D., Gojis, O., Ellis, I. O., Green, A. R., et al. (2012). Differential oestrogen receptor binding is associated with clinical outcome in breast cancer. Nature 481, 389-393. https://doi.org/10.1038/nature10730.
Schep, A. N., Wu, B., Buenrostro, J. D., and Greenleaf, W. J. (2017). chromVAR: inferring transcription-factor-associated accessibility from single-cell epigenomic data. Nat. Methods 14, 975-978. https://doi.org/10.1038/nmeth.4401.
Schultheiß, C., Willscher, E., Paschold, L., Gottschick, C., Klee, B., Henkes, S.-S., Bosurgi, L., Dutzmann, J., Sedding, D., Frese, T., et al. (2022). The IL-1β, IL-6, and TNF cytokine triad is associated with post-acute sequelae of COVID-19. Cell Rep. Med. 3, 100663. https://doi.org/10.1016/j.xcrm.2022.100663.
Schultze, J. L., Mass, E., and Schlitzer, A. (2019). Emerging Principles in Myelopoiesis at Homeostasis and during Infection and Inflammation. Immunity 50, 288-301. https://doi.org/10.1016/j.immuni.2019.01.019.
Seita, J., and Weissman, I. L. (2010). Hematopoietic stem cell: self-renewal versus differentiation. Wiley Interdiscip. Rev. Syst. Biol. Med. 2, 640-653. https://doi.org/10.1002/wsbm.86.
Song, R., Gao, Y., Dozmorov, I., Malladi, V., Saha, I., McDaniel, M. M., Parameswaran, S., Liang, C., Arana, C., Zhang, B., et al. (2021). IRF1 governs the differential interferon-stimulated gene responses in human monocytes and macrophages by regulating chromatin accessibility. Cell Rep. 34, 108891. https://doi.org/10.1016/j.celrep.2021.108891.
Stuart, T., Srivastava, A., Madad, S., Lareau, C. A., and Satija, R. (2021). Single-cell chromatin state analysis with Signac. Nat. Methods 18, 1333-1341. https://doi.org/10.1038/s41592-021-01282-5.
Subramanian, A., P. Tamayo, V. K. Mootha, S. Mukherjee, B. L. Ebert, M. A. Gillette, A. Paulovich, S. L. Pomeroy, T. R. Golub, E. S. Lander, and J. P. Mesirov. 2005. Gene set enrichment analysis: a knowledge-based approach for interpreting genome-wide expression profiles. Proc Natl Acad Sci USA 102:15545-15550.
Sudre, C. H., Murray, B., Varsavsky, T., Graham, M. S., Penfold, R. S., Bowyer, R. C., Pujol, J. C., Klaser, K., Antonelli, M., Canas, L. S., et al. (2021). Attributes and predictors of long COVID. Nat. Med. 27, 626-631. https://doi.org/10.1038/s41591-021-01292-y.
Tenforde, M. W., and Morris, S. B. (2021). Multisystem inflammatory syndrome in adults: coming into focus. Chest 159, 471-472. https://doi.org/10.1016/j.chest.2020.09.097.
Thibodeau, A., Eroglu, A., McGinnis, C. S., Lawlor, N., Nehar-Belaid, D., Kursawe, R., Marches, R., Conrad, D. N., Kuchel, G. A., Gartner, Z. J., et al. (2021). AMULET: a novel read count-based method for effective multiplet detection from single nucleus ATAC-seq data. Genome Biol. 22, 252. https://doi.org/10.1186/s13059-021-02469-x.
Ucar, D., and Karakaslar, E. O. (2021). cinaR: A comprehensive R package for the dif-ferential analyses and functional interpretation of ATAC-seq data. BioRxiv https://doi.org/10.1101/2021.03.05.434143.
Wang, D., Hu, B., Hu, C., Zhu, F., Liu, X., Zhang, J., Wang, B., Xiang, H., Cheng, Z., Xiong, Y., et al. (2020a). Clinical Characteristics of 138 Hospitalized Patients With 2019 Novel Coronavirus-Infected Pneumonia in Wuhan, China. JAMA 323, 1061-1069. https://doi.org/10.1001/jama.2020.1585.
Wang, J., Jiang, M., Chen, X., and Montaner, L. J. (2020b). Cytokine storm and leukocyte changes in mild versus severe SARS-CoV-2 infection: Review of 3939 COVID-19 patients in China and emerging pathogenesis and therapy concepts. J. Leukoc. Biol. 108, 17-41. https://doi.org/10.1002/JLB.3COVR0520-272R.
Wimmers, F., Donato, M., Kuo, A., Ashuach, T., Gupta, S., Li, C., Dvorak, M., Foecke, M. H., Chang, S. E., Hagan, T., et al. (2021). The single-cell epigenomic and transcriptional landscape of immunity to influenza vaccination. Cell 184, 3915-3935.e21. https://doi.org/10.1016/j.cell.2021.05.039.
Wolock, S. L., Lopez, R., and Klein, A. M. (2019). Scrublet: Computational Identification of Cell Doublets in Single-Cell Transcriptomic Data. Cell Syst. 8, 281-291.e9. https://doi.org/10.1016/j.cels.2018.11.005.
Xie, Y., Bowe, B., and AI-Aly, Z. (2021). Burdens of post-acute sequelae of COVID-19 by severity of acute infection, demographics and health status. Nat. Commun. 12, 6571. https://doi.org/10.1038/s41467-021-26513-3.
Xiong, Y., Liu, Y., Cao, L., Wang, D., Guo, M., Jiang, A., Guo, D., Hu, W., Yang, J., Tang, Z., et al. (2020). Transcriptomic characteristics of bronchoalveolar lavage fluid and peripheral blood mononuclear cells in COVID-19 patients. Emerg. Microbes Infect. 9, 761-770. https://doi.org/10.1080/22221751.2020.1747363.
Yang, D., Chu, H., Hou, Y., Chai, Y., Shuai, H., Lee, A. C.-Y., Zhang, X., Wang, Y., Hu, B., Huang, X., et al. (2020). Attenuated Interferon and Proinflammatory Response in SARS-CoV-2-Infected Human Dendritic Cells Is Associated With Viral Antagonism of STAT1 Phosphorylation. J. Infect. Dis. 222, 734-745. https://doi.org/10.1093/infdis/jiaa356.
Yu, G., Wang, L.-G., Han, Y., and He, Q.-Y. (2012). clusterProfiler: an R package for comparing biological themes among gene clusters. OMICS 16, 284-287. https://doi.org/10.1089/omi.2011.0118.
Yu, G., Wang, L.-G., and He, Q.-Y. (2015). ChlPseeker: an R/Bioconductor package for ChIP peak annotation, comparison and visualization. Bioinformatics 31, 2382-2383. https://doi.org/10.1093/bioinformatics/btv145.
Zhang, Q., Bastard, P., Liu, Z., Le Pen, J., Moncada-Velez, M., Chen, J., Ogishi, M., Sabli, I. K. D., Hodeib, S., Korol, C., et al. (2020). Inborn errors of type I IFN immunity in patients with life-threatening COVID-19. Science 370. https://doi.org/10.1126/science.abd4570.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol. 9, R137. https://doi.org/10.1186/gb-2008-9-9-r137.
Zhou, R., To, K. K.-W., Wong, Y.-C., Liu, L., Zhou, B., Li, X., Huang, H., Mo, Y., Luk, T.-Y., Lau, T. T.-K., et al. (2020). Acute SARS-CoV-2 Infection Impairs Dendritic Cell and T Cell Responses. Immunity 53, 864-877.e5. https://doi.org/10.1016/j.immuni.2020.07.026.
Zhu, N., Zhang, D., Wang, W., Li, X., Yang, B., Song, J., Zhao, X., Huang, B., Shi, W., Lu, R., et al. (2020). A Novel Coronavirus from Patients with Pneumonia in China, 2019. N. Engl. J. Med. 382, 727-733. https://doi.org/10.1056/NEJMoa2001017.
(2021). Ready-to-Use Curated Gene Sets for “cinaR” [R package cinaRgenesets version 0.1.1] https://cran.r-project.org/web/packages/cinaRgenesets/index.html.

What is claimed is:

1. A method of characterizing cellular molecular features and/or functional characteristics in an enriched population of rare circulating cells from peripheral blood, the method comprising:
isolating one or more types of rare circulating cells from peripheral blood or from peripheral blood mononuclear cells (PBMC) from a peripheral blood sample;
enriching the one or more types of rare circulating cells in the PBMC and/or in the peripheral blood sample, thereby providing an enriched population of rare circulating cells from the peripheral blood and/or PBMC;
acquiring single cell and/or bulk transcriptomic, genetic, and/or protein expression, and/or metabolic, epigenomic, and/or other functional assay data for the enriched population of rare circulating cells;
analyzing the enriched rare circulating cell transcriptomic, genetic, and/or protein expression, and/or metabolic, epigenomic, and/or other functional assay data to identify cellular molecular features and/or functional characteristics; and
generating an output comprising transcriptional, genetic, protein, metabolic, epigenomic, and/or functional characteristic signatures, thereby characterizing cellular molecular features and/or functional characteristics for the one or more types of rare circulating cells.

2. The method of claim 1, wherein rare circulating cell enrichment comprises either antibody-conjugated bead-based enrichment or FACS sorting, or sequential antibody-conjugated bead-based enrichment and FACS sorting.

3. The method of claim 1 or claim 2, wherein rare circulating cell enrichment comprises FACS-sorting rare circulating cells into one or more tubes prior to cell isolation.

4. The method of any preceding claim, wherein rare circulating cell enrichment comprises pooling multiple samples into a single assay tube and demultiplexing after analysis (in silico) based on oligo-conjugated antibody-based demultiplexing or genotype (SNP) based demultiplexing using genetic variance between individuals.

5. The method of any preceding claim, wherein the enriched population of rare circulating cells are introduced or re-introduced into a sample comprising peripheral blood and/or PBMC.

6. The method of any preceding claim, wherein the peripheral blood and/or PBMC comprises one or more peripheral hematopoietic stem and progenitor cell (pHSPC), CD14+ monocyte (CD14 M.), CD16+ monocyte (CD16 M.), CD34+ HSPC, CD34− HSPC, B cell (B), CD4+ T cell (CD4), CD8+ T cell (CD8), dendritic cell (DC), natural killer cell (NK), plasma B cell (PC), plasmacytoid dendritic cells (pDC), hematopoietic stem cells/multipotent progenitor cell (HSC/MPP), lymphoid-primed multipotent progenitor cell (LMPP), megakaryocyte-erythroid progenitor cell (MEP), erythroid progenitor cell (Ery), granulocyte-monocyte progenitor cell (GMP), basophil-eosinophil-mast cell progenitor cell (BEM), or common myeloid progenitor (CMP).

7. The method of any preceding claim, wherein the rare circulating cell is a peripheral hematopoietic stem and progenitor cell (pHSPC), CD14+ monocyte (CD14 M.), CD16+ monocyte (CD16 M.), B cell (B), CD4+ T cell (CD4), CD8+ T cell (CD8), dendritic cell (DC), natural killer cell (NK), plasma B cell (PC), plasmacytoid dendritic cells (pDC), hematopoietic stem cells/multipotent progenitor cell (HSC/MPP), lymphoid-primed multipotent progenitor cell (LMPP), megakaryocyte-erythroid progenitor cell (MEP), erythroid progenitor cell (Ery), granulocyte-monocyte progenitor cell (GMP), basophil-eosinophil-mast cell progenitor cell (BEM), or common myeloid progenitor (CMP).

8. The method of any preceding claim, wherein the rare circulating cell is a pHSPC or pDC.

9. The method of claim 8, wherein the pHSPC is a CD34+ or CD34− pHSPC.

10. The method of any preceding claim, wherein the peripheral blood sample is obtained directly from a subject or is from cyroperserved PBMC and/or cryopreserved peripheral blood.

11. The method of any preceding claim, wherein acquiring the single cell and/or bulk transcriptomic, genetic, and/or protein expression, and/or metabolic, epigenomic, and/or other functional assay data comprises one or more bulk and/or single cell assay.

12. The method of claim 11, wherein the bulk and/or single cell assay comprises bulk and/or single cell RNA and/or ATACseq analysis.

13. The method of claim 11, wherein acquiring the single cell and/or bulk transcriptomic, genetic, and/or protein expression, and/or metabolic, epigenomic, and/or other functional assay data comprises one or more single cell assay and is combined with one or more single cell-based workflows.

14. The method of any preceding claim, further comprising parallel sample preparation and scale up enabled by pooling of multiple samples and demultiplexing after analysis (in silico) based on oligo-conjugated antibody-based demultiplexing or genotype (SNP) based demultiplexing using genetic variance between individuals.

15. The method of any preceding claim, further comprising subject genome sequencing to generate a reference genotype for genotype-based demultiplexing of single cell datasets from pooled samples.

16. The method of claim 15, wherein genome sequencing comprises whole genome sequencing, exome sequencing, bulk ATACseq, and/or SNP microarray.

17. The method of any preceding claim, wherein analyzing the enriched rare circulating cells comprises analyzing expression of one or more of protein, mRNA, DNA (sequence or post-translational modifications), chromatin (e.g. histone modifications, accessibility, 3D structure/looping, etc.), metabolites, and/or lipids.

18. The method of claim 17, wherein analyzing the enriched rare circulating cells comprises analyzing chromatin, DNA, mRNA expression, and/or ATAC-seq data.

19. The method of claim 18, wherein analyzing the enriched rare circulating cell mRNA and assay for transposase-accessible chromatin sequencing (ATAC-seq) data comprises combined single cell mRNA/ATAC-seq data processing; UMAP visualization; single cell and/or bulk ATAC-seq; demultiplexing; and/or identifying differentially accessible regions, differentially expressed genes, and/or ATAC peak-gene/transcript associations.

20. The method of any preceding claim, wherein transcriptional, genetic, protein, and/or epigenomic signatures are determined by gene ontology (GO) analysis.

21. The method of any preceding claim, wherein analyzing the enriched rare circulating cells comprises combined single nuclei (sn) RNA and assay for transposase-accessible chromatin sequencing (ATAC-seq) (chromium single cell multiome ATAC+ gene expression) for PBMC, sorted PBMC subset “bulk” ATAC-seq, multiplexed immunoassay-based quantitation of plasma proteins, and/or immunophenotyping by flow cytometry.

22. The method of any preceding claim, wherein the enriched rare circulating cells have differential enrichment of epigenetic and transcriptional signatures associated with antigen presentation, activation, differentiation, and/or anti-viral responses.

23. The method of any preceding claim, wherein the cellular molecular features and/or functional characteristics of the enriched rare circulating cells comprise increased granulo- and myelopoiesis in pHSPC, and/or monocyte phenotypes of inflammation, migration, and differentiation, and/or altered proportions or phenotypes of pHSPC subsets related to changes in hematopoiesis.

24. The method of claim 23, wherein the increased pHSPC subsets related to changes in hematopoiesis comprise HSC, MPP, GMP, CMP, BEM, MEP, Ery, and/or LMPP.

25. The method of any preceding claim, wherein the cellular molecular features and/or functional characteristics of the enriched rare circulating cells and/or pHSPC-enriched PBMC are used in a diagnostic assay.

26. The method of any preceding claim, wherein the method characterizes a single rare circulating cell.

27. The method of any preceding claim, the method further comprising using the enriched rare circulating cells in one or more functional assay.

28. The method of claim 27, wherein the functional assay comprises a differentiation potential (e.g. colony forming assay (CFA)), stimulation responsiveness (e.g. cytokine secretion/production), metabolism (e.g. oxygen consumption), migration/motility, histone modification, and/or DNA methylation assay.

29. The method of any preceding claim, wherein the cellular molecular features and/or functional characteristics of the enriched rare circulating cells are compared to the cellular molecular features and/or functional characteristics from one or more stem cells from a bone marrow sample.

30. The method of claim 29, wherein the rare circulating cells are isolated from a subject, and wherein the bone marrow stem cells are from the same subject.

31. The method of claim 29, wherein the rare circulating cells and the bone marrow stem cells are from different clinical groups.

32. The method of any preceding claim, wherein the characterization of cellular molecular features and/or functional characteristics for the enriched rare circulating cells are used in place of or in addition to characterization of stem cells obtained from bone marrow.

33. The method of any preceding claim, wherein the characterization of cellular molecular features and/or functional characteristics for the enriched rare circulating cells are used in place of characterization of stem cells obtained from bone marrow aspiration and/or biopsy.

34. The method of any preceding claim, wherein the characterization of cellular molecular features and/or functional characteristics for the enriched rare circulating cells is used in place of or in combination with a diagnostic assay based on characterization of one or more stem cells obtained from bone marrow aspiration and/or biopsy.

35. The method of any preceding claim, wherein the characterization of cellular molecular features and/or functional characteristics for the enriched rare circulating cells is used in place of a diagnostic assay based on characterization of stem cells obtained from bone marrow aspiration and/or biopsy in diagnosis and/or treatment of inflammatory diseases, types of cancer (e.g. lymphoma, leukemia, myeloma, metastatic cancer), metabolic diseases (e.g. anemia, hemochromatosis), and/or blood disorders/conditions (leukopenia, leukocytosis, thrombocytopenia, thrombocytosis, pancytopenia, polycythemia).

36. The method of any preceding claim, wherein the sample is from a subject post-COVID-19 infection and having one or more symptoms of long covid, and wherein the rare circulating cells comprise one or more pHSPC.

37. The method of claim 36, wherein the characterization of cellular molecular features and/or functional characteristics for the enriched rare circulating cells is used for single-cell profiling of human pHSPC from peripheral blood post-COVID-19 infection.

38. The method of claim 36, wherein the sample is from a subject post-COVID-19 infection and having one or more symptoms of long covid, and wherein the characterization of cellular molecular features and/or functional characteristics for the pHSPC comprises analysis of pHSPC transcriptomic, epigenomic, and/or protein data.

39. The method of claim 38, wherein the pHSPC transcriptomic, epigenomic, and/or protein data is from 1 week, 2 weeks, 3 weeks, 1 month, 2 months, 3 months, 6 months, 9 months, 12 months, or longer after COVID-19 infection.

40. The method of claim 38, wherein the pHSPC transcriptomic, epigenomic, and/or protein data is from 2-4 months post hospital admission, or from 5-12 months after COVID-19 infection.

41. The method of claim 38, wherein the characterization of cellular molecular features and/or functional characteristics for the pHSPC post-COVID-19 infection comprises pHSPC transcriptional and epigenetic signatures.

42. The method of claim 41, wherein pHSPC transcriptional and epigenetic signatures post-COVID-19 infection comprise enrichment for one or more inflammatory genes or genetic regulatory elements; one or more genes or genetic regulatory elements related to antigen presentation, activation, differentiation, and/or anti-viral responses; and/or gene enrichment and/or enrichment of accessibility at promoters, enhancers, and/or differently accessible regions (DAR).

43. The method of claim 42, wherein the pHSPC transcriptional and epigenetic signatures post-COVID-19 infection comprise enrichment for one or more inflammatory genes; IL-6R signaling genes; genes related to antigen presentation, activation, differentiation, and/or anti-viral responses; genes related to differentiation, migration, activation, and cytokine-mediated signaling; genes related to myeloid differentiation, activation, and cytokine production; genes related to programs of myeloid dendritic cell activation; genes related to platelet activation; and/or genes related to neutrophil/GMP/activation; chromatin accessibility genes encoding cytokines, adhesion molecules, and/or differentiation factors; and/or wherein the PBMC transcriptional and epigenetic signatures post-COVID-19 infection comprise dysregulation of hematopoiesis and genes linked to granulopoiesis and myelopoiesis; reduced expression of negative feedback factors; increased chromatin accessibility at chemokines; chemokine receptor genes; interferon stimulated genes; and/or immunomodulatory genes.

44. The method of claim 43, wherein one or more inflammatory genes comprise S100A12, CTSC, IL6, CD28, NLRP12, IRF1, STAT1, NFKB1, NFKBIA, PPARG, IL1RAP, and/or MAPKAPK2; genes related to IL-6R signaling comprise CEBPb, STAT3, and/or CRP; late enriched monocyte genes comprise M.SC3; genes related to antigen presentation, activation, differentiation, and/or anti-viral responses comprise CD74, LGMN, B2M, IFI30, HLA, LYZ, CD14, S100As, and/or IL1B; the genes related to differentiation, migration, activation, and cytokine-mediated signaling comprise PTPRC, ITGAM, CCL26, IL1RL2, and/or IFI16; the genes related to myeloid differentiation, activation, and cytokine production comprise IKZF1, IL4R, RARA, STAT3, and/or KLF13; the genes related to programs of myeloid dendritic cell activation comprise RELB, CD2, CAMK4, and/or SLAMF1; the genes related to platelet activation comprise GP1BB, PDGFB, CD40, and/or MYH9; the genes related to neutrophil/GMP/activation comprise S100A9, S100A8, CAMK1D, and/or CD74; the chromatin accessibility genes encoding cytokines comprise CCL3, IL10, and/or IFNG; the chromatin accessibility genes encoding adhesion molecules comprise CD1D, DOCK5, ADAM9, and/or ITGAL; and the chromatin accessibility genes encoding differentiation factors comprise KLF13, CREB1, PRKCA, and/or FOXP1; the genes linked to granulopoiesis and myelopoiesis comprise CD14, KLF2, CEBPD, and CCL5; the negative feedback factors comprise DUSP1 and/or NFKBIA; the chemokines comprise CCL4 and/or CXCL8; the chemokine receptors comprise CCR4, CCR6, and/or CXCR3; the interferon stimulated genes comprise NLRC5, SOCS1, and/or IFITM1; and/or the immunomodulatory genes comprise METRNL, NLRC3, KLF4, and/or TNFAIP3.

45. The method of claim 41, wherein the characterization of cellular molecular features and/or functional characteristics for the pHSPC post-COVID-19 infection comprises enrichment for chromatin binding or inferred chromatin binding (based on motif enrichment in DAR and/or footprints) of NRF1, STAT3, NFkB, CEBPb, AP-1, IRF1, IRF2, IRF3, IRF4, IRF5, IRF6, IRF7, IRF8, and/or CTCF.

46. The method of claim 41, wherein pHSPC transcriptional and epigenetic signatures post-COVID-19 infection comprise increased pHSPC subsets related to changes in hematopoiesis.

47. The method of claim 46, wherein the increased HSPC subsets related to changes in hematopoiesis comprise HSC, MPP, GMP, CMP, BEM, MEP, Ery, and/or LMPP.

48. The method of any of claims 36-47, wherein the characterization of cellular molecular features and/or functional characteristics for the pHSPC post-COVID-19 infection comprises increased granulo- and myelopoiesis in pHSPC, and monocyte phenotypes of inflammation, migration, and differentiation.

49. The method of any of claims 41-48, the method further comprising treating the subject having long covid symptoms by:
identifying one or more therapeutic targets based on the pHSPC transcriptional and epigenetic signatures; and
treating the subject by administering a treatment directed to the one or more targets identified from the pHSPC transcriptional and epigenetic signatures.

50. The method of claim 49, wherein the therapeutic target comprises IL-6, IL-6R, IL-1, IL-12/23, IL-17, IL-23, IL-4/13, TNF, JAK, and/or another cytokine.

51. The method of claim 50, wherein the therapeutic target comprises IL-6, and wherein the treatment comprises an IL-6R blocking antibody, and/or G-CSF, GM-CSF, and/or chemokine/cytokine targeting.

52. The method of claim 51, wherein the IL-6R blocking antibody comprises Tocilizumab and/or Sarilumab.

53. The method of claim 50, wherein the treatment comprises an anti-inflammatory biologic and/or a steroid.

54. The method of claim 1, wherein the characterization of cellular molecular features and/or functional characteristics for the enriched rare circulating cells is associated with clinical data and laboratory results to identify one or more mechanisms of disease, biomarkers, and/or therapeutic targets related to changes in hematopoiesis.

55. The method of claim 54, wherein a subject is subsequently treated by administering a treatment directed to the one or more targets identified from the association of the cellular molecular features and/or functional characteristics for the enriched rare circulating cells with clinical data and laboratory results.

56. A method of enriching rare circulating cells from peripheral blood, wherein the method comprises:
isolating one or more types of rare circulating cells from peripheral blood or from peripheral blood mononuclear cells (PBMC) from a peripheral blood sample; and
enriching the one or more types of rare circulating cell in the PBMC and/or in the peripheral blood sample, thereby providing an enriched population of rare circulating cells from the peripheral blood and/or PBMC.

57. The method of claim 56, wherein rare circulating cell enrichment comprises either antibody-conjugated bead-based enrichment or FACS sorting, or sequential antibody-conjugated bead-based enrichment and FACS sorting.

58. The method of claim 56 or claim 57, wherein rare circulating cell enrichment comprises FACS-sorting rare circulating cells into one or more tubes prior to cell isolation.

59. The method of any of claims 56-58, wherein rare circulating cell enrichment comprises pooling multiple samples into a single assay tube and demultiplexing after analysis (in silico) based on oligo-conjugated antibody-based demultiplexing or genotype (SNP) based demultiplexing using genetic variance between individuals.

60. The method of any of claims 56-59, wherein the enriched population of rare circulating cells are introduced or re-introduced into a sample comprising peripheral blood and/or PBMC.

61. The method of any of claims 56-60, wherein the peripheral blood and/or PBMC comprises one or more peripheral hematopoietic stem and progenitor cell (pHSPC), CD14+ monocyte (CD14 M.), CD16+ monocyte (CD16 M.), CD34+ HSPC, CD34− HSPC, B cell (B), CD4+ T cell (CD4), CD8+ T cell (CD8), dendritic cell (DC), natural killer cell (NK), plasma B cell (PC), plasmacytoid dendritic cells (pDC), hematopoietic stem cells/multipotent progenitor cell (HSC/MPP), lymphoid-primed multipotent progenitor cell (LMPP), megakaryocyte-erythroid progenitor cell (MEP), erythroid progenitor cell (Ery), granulocyte-monocyte progenitor cell (GMP), basophil-eosinophil-mast cell progenitor cell (BEM), or common myeloid progenitor (CMP).

62. The method of any of claims 56-61, wherein the rare circulating cell is a peripheral hematopoietic stem and progenitor cell (pHSPC), CD14+ monocyte (CD14 M.), CD16+ monocyte (CD16 M.), B cell (B), CD4+ T cell (CD4), CD8+ T cell (CD8), dendritic cell (DC), natural killer cell (NK), plasma B cell (PC), plasmacytoid dendritic cells (pDC), hematopoietic stem cells/multipotent progenitor cell (HSC/MPP), lymphoid-primed multipotent progenitor cell (LMPP), megakaryocyte-erythroid progenitor cell (MEP), erythroid progenitor cell (Ery), granulocyte-monocyte progenitor cell (GMP), basophil-eosinophil-mast cell progenitor cell (BEM), or common myeloid progenitor (CMP).

63. The method of any of claims 56-62, wherein the rare circulating cell is a pHSPC or pDC.

64. The method of claim 63, wherein the pHSPC is a CD34+ or CD34− pHSPC.

65. The method of any of claims 56-64, wherein the peripheral blood sample is obtained directly from a subject or is from cryopreserved PBMC and/or cryopreserved peripheral blood.

66. The method of any of claims 56-65, further comprising analyzing the enriched rare circulating cells by downstream analysis of cellular molecular features and/or cell functional characteristics.

67. The method of claim 66, wherein downstream analysis of cellular molecular features and/or cell functional characteristics comprises:
acquiring single cell and/or bulk transcriptomic, genetic, and/or protein expression, and/or metabolic, epigenomic, and/or other functional assay data for the enriched population of rare circulating cells;
analyzing the rare circulating cell transcriptomic, genetic, and/or protein expression, and/or metabolic, epigenomic, and/or other functional assay data to identify cellular molecular features and/or functional characteristics; and
generating an output comprising transcriptional, genetic, protein, metabolic, epigenomic, and/or functional characteristic signatures for the one or more types of rare circulating cells.

68. The method of claim 67, wherein the cellular molecular features and/or functional characteristics comprise transcriptional and/or epigenetic signatures for the enriched rare circulating cell.

69. The method of claim 67, wherein acquiring the single cell and/or bulk transcriptomic, genetic, and/or protein expression, and/or metabolic, epigenomic, and/or other functional assay data comprises one or more bulk and/or single cell assay.

70. The method of claim 69, wherein the bulk and/or single cell assay comprises bulk and/or single cell RNA and/or ATACseq analysis.

71. The method of claim 69, wherein acquiring the single cell and/or bulk transcriptomic, genetic, and/or protein expression, and/or metabolic, epigenomic, and/or other functional assay data comprises one or more single cell assay and is combined with one or more single cell-based workflows.

72. The method of claim 67, further comprising parallel sample preparation and scale up enabled by pooling of multiple samples and demultiplexing after analysis (in silico) based on oligo-conjugated antibody-based demultiplexing or genotype (SNP) based demultiplexing using genetic variance between individuals.

73. The method of claim 67, further comprising subject genome sequencing to generate a reference genotype for genotype-based demultiplexing of single cell datasets from pooled samples.

74. The method of claim 73, wherein genome sequencing comprises whole genome sequencing, exome sequencing, bulk ATACseq, and/or SNP microarray.

75. The method of claim 67, wherein analyzing the enriched rare circulating cells comprises analyzing expression of one or more of protein, mRNA, DNA (sequence or post-translational modifications), chromatin (e.g. histone modifications, accessibility, 3D structure/looping, etc.), metabolites, and/or lipids.

76. The method of claim 75, wherein analyzing the enriched rare circulating cells comprises analyzing chromatin, DNA, mRNA expression, and/or ATAC-seq data.

77. The method of claim 76, wherein analyzing the enriched rare circulating cell mRNA and assay for transposase-accessible chromatin sequencing (ATAC-seq) data comprises combined single cell mRNA/ATAC-seq data processing; UMAP visualization; single cell and/or bulk ATAC-seq; demultiplexing; and/or identifying differentially accessible regions, differentially expressed genes, and/or ATAC peak-gene/transcript associations.

78. The method of claim 67, wherein transcriptional, genetic, protein, and/or epigenomic signatures are determined by gene ontology (GO) analysis.

79. The method of claim 67, wherein analyzing the enriched rare circulating cells comprises combined single nuclei (sn) RNA and assay for transposase-accessible chromatin sequencing (ATAC-seq) (chromium single cell multiome ATAC+ gene expression) for PBMC, sorted PBMC subset “bulk” ATAC-seq, multiplexed immunoassay-based quantitation of plasma proteins, and/or immunophenotyping by flow cytometry.

80. The method of claim 67, wherein the enriched rare circulating cells have differential enrichment of epigenetic and transcriptional signatures associated with antigen presentation, activation, differentiation, and/or anti-viral responses.

81. The method of claim 67, wherein the cellular molecular features and/or functional characteristics of the enriched rare circulating cells comprise increased granulo- and myelopoiesis in pHSPC, and/or monocyte phenotypes of inflammation, migration, and differentiation, and/or altered proportions or phenotypes of pHSPC subsets related to changes in hematopoiesis.

82. The method of claim 81, wherein the increased pHSPC subsets related to changes in hematopoiesis comprise HSC, MPP, GMP, CMP, BEM, MEP, Ery, and/or LMPP.

83. The method of any of claims 67-82, wherein the cellular molecular features and/or functional characteristics of the enriched rare circulating cells and/or pHSPC-enriched PBMC are used in a diagnostic assay.

84. The method of any of claims 67-83, wherein the method characterizes a single rare circulating cell.

85. The method of any of claims 56-84, the method further comprising using the enriched rare circulating cells in one or more functional assay.

86. The method of claim 85, wherein the functional assay comprises a differentiation potential (e.g. colony forming assay (CFA)), stimulation responsiveness (e.g. cytokine secretion/production), metabolism (e.g. oxygen consumption), migration/motility, histone modification, and/or DNA methylation assay.

87. The method of any of claims 67-86, wherein the cellular molecular features and/or functional characteristics of the enriched rare circulating cells are compared to the cellular molecular features and/or functional characteristics from one or more stem cells from a bone marrow sample.

88. The method of claim 87, wherein the rare circulating cells are isolated from a subject, and wherein the bone marrow stem cells are from the same subject.

89. The method of claim 87, wherein the rare circulating cells and the bone marrow stem cells are from different clinical groups.

90. The method of any claims 67-89, wherein the characterization of cellular molecular features and/or functional characteristics for the enriched rare circulating cells are used in place of or in addition to characterization of stem cells obtained from bone marrow.

91. The method of any of claims 67-90, wherein the characterization of cellular molecular features and/or functional characteristics for the enriched rare circulating cells are used in place of characterization of stem cells obtained from bone marrow aspiration and/or biopsy.

92. The method of any of claims 67-91, wherein the characterization of cellular molecular features and/or functional characteristics for the enriched rare circulating cells is used in place of or in combination with a diagnostic assay based on characterization of one or more stem cells obtained from bone marrow aspiration and/or biopsy.

93. The method of any of claims 67-92, wherein the characterization of cellular molecular features and/or functional characteristics for the enriched rare circulating cells is used in place of a diagnostic assay based on characterization of stem cells obtained from bone marrow aspiration and/or biopsy in diagnosis and/or treatment of inflammatory diseases, types of cancer (e.g. lymphoma, leukemia, myeloma, metastatic cancer), metabolic diseases (e.g. anemia, hemochromatosis), and/or blood disorders/conditions (leukopenia, leukocytosis, thrombocytopenia, thrombocytosis, pancytopenia, polycythemia).

94. The method of any of claims 67-93, wherein the sample is from a subject post-COVID-19 infection and having one or more symptoms of long covid, and wherein the rare circulating cells comprise one or more pHSPC.

95. The method of claim 94, wherein the characterization of cellular molecular features and/or functional characteristics for the enriched rare circulating cells is used for single-cell profiling of human pHSPC from peripheral blood post-COVID-19 infection.

96. The method of claim 94, wherein the sample is from a subject post-COVID-19 infection and having one or more symptoms of long covid, and wherein the characterization of cellular molecular features and/or functional characteristics for the pHSPC comprises analysis of pHSPC transcriptomic, epigenomic, and/or protein data.

97. The method of claim 96, wherein the pHSPC transcriptomic, epigenomic, and/or protein data is from 1 week, 2 weeks, 3 weeks, 1 month, 2 months, 3 months, 6 months, 9 months, 12 months, or longer after COVID-19 infection.

98. The method of claim 96, wherein the pHSPC transcriptomic, epigenomic, and/or protein data is from 2-4 months post hospital admission, or from 5-12 months after COVID-19 infection.

99. The method of claim 96, wherein the characterization of cellular molecular features and/or functional characteristics for the pHSPC post-COVID-19 infection comprises pHSPC transcriptional and epigenetic signatures.

100. The method of claim 99, wherein pHSPC transcriptional and epigenetic signatures comprise enrichment for one or more inflammatory genes or genetic regulatory elements and/or one or more genes or genetic regulatory elements related to antigen presentation, activation, differentiation, and/or anti-viral responses; and/or gene enrichment and/or enrichment of accessibility at promoters, enhancers, and/or differently accessible regions (DAR).

101. The method of claim 100, wherein the pHSPC transcriptional and epigenetic signatures post-COVID-19 infection comprise enrichment for one or more inflammatory genes; IL-6R signaling genes; genes related to antigen presentation, activation, differentiation, and/or anti-viral responses; genes related to differentiation, migration, activation, and cytokine-mediated signaling; genes related to myeloid differentiation, activation, and cytokine production; genes related to programs of myeloid dendritic cell activation; genes related to platelet activation; and/or genes related to neutrophil/GMP/activation; chromatin accessibility genes encoding cytokines, adhesion molecules, and/or differentiation factors; and/or wherein the PBMC transcriptional and epigenetic signatures post-COVID-19 infection comprise dysregulation of hematopoiesis and genes linked to granulopoiesis and myelopoiesis; reduced expression of negative feedback factors; increased chromatin accessibility at chemokines; chemokine receptor genes; interferon stimulated genes; and/or immunomodulatory genes.

102. The method of claim 101, wherein one or more inflammatory genes comprise S100A12, CTSC, IL6, CD28, NLRP12, IRF1, STAT1, NFKB1, NFKBIA, PPARG, IL1RAP, and/or MAPKAPK2; genes related to IL-6R signaling comprise CEBPb, STAT3, and/or CRP; late enriched monocyte genes comprise M.SC3; genes related to antigen presentation, activation, differentiation, and/or anti-viral responses comprise CD74, LGMN, B2M, IFI30, HLA, LYZ, CD14, S100As, and/or IL1B; the genes related to differentiation, migration, activation, and cytokine-mediated signaling comprise PTPRC, ITGAM, CCL26, IL1RL2, and/or IFI16; the genes related to myeloid differentiation, activation, and cytokine production comprise IKZF1, IL4R, RARA, STAT3, and/or KLF13; the genes related to programs of myeloid dendritic cell activation comprise RELB, CD2, CAMK4, and/or SLAMF1; the genes related to platelet activation comprise GP1BB, PDGFB, CD40, and/or MYH9; the genes related to neutrophil/GMP/activation comprise S100A9, S100A8, CAMK1D, and/or CD74; the chromatin accessibility genes encoding cytokines comprise CCL3, IL10, and/or IFNG; the chromatin accessibility genes encoding adhesion molecules comprise CD1D, DOCK5, ADAM9, and/or ITGAL; and the chromatin accessibility genes encoding differentiation factors comprise KLF13, CREB1, PRKCA, and/or FOXP1; the genes linked to granulopoiesis and myelopoiesis comprise CD14, KLF2, CEBPD, and CCL5; the negative feedback factors comprise DUSP1 and/or NFKBIA; the chemokines comprise CCL4 and/or CXCL8; the chemokine receptors comprise CCR4, CCR6, and/or CXCR3; the interferon stimulated genes comprise NLRC5, SOCS1, and/or IFITM1; and/or the immunomodulatory genes comprise METRNL, NLRC3, KLF4, and/or TNFAIP3.

103. The method of claim 99, wherein the characterization of cellular molecular features and/or functional characteristics for the pHSPC post-COVID-19 infection comprises enrichment for chromatin binding or inferred chromatin binding (based on motif enrichment in DAR and/or footprints) of NRF1, STAT3, NFkB, CEBPb, AP-1, IRF1, IRF2, IRF3, IRF4, IRF5, IRF6, IRF7, IRF8, and/or CTCF.

104. The method of claim 99, wherein pHSPC transcriptional and epigenetic signatures post-COVID-19 infection comprise increased pHSPC subsets related to changes in hematopoiesis.

105. The method of claim 104, wherein the increased HSPC subsets related to changes in hematopoiesis comprise HSC, MPP, GMP, CMP, BEM, MEP, Ery, and/or LMPP.

106. The method of any of claims 94-105, wherein the characterization of cellular molecular features and/or functional characteristics for the pHSPC post-COVID-19 infection comprises increased granulo- and myelopoiesis in pHSPC, and monocyte phenotypes of inflammation, migration, and differentiation.

107. The method of any of claims 94-106, the method further comprising treating the subject having long covid symptoms by:
identifying one or more therapeutic targets based on the pHSPC transcriptional and epigenetic signatures; and
treating the subject by administering a treatment directed to the one or more targets identified from the pHSPC transcriptional and epigenetic signatures.

108. The method of claim 107, wherein the therapeutic target comprises IL-6, IL-6R, IL-1, IL-12/23, IL-17, IL-23, IL-4/13, TNF, JAK, and/or another cytokine.

109. The method of claim 108, wherein the therapeutic target comprises IL-6, and wherein the treatment comprises an IL-6R blocking antibody, and/or G-CSF, GM-CSF, and/or chemokine/cytokine targeting.

110. The method of claim 109, wherein the IL-6R blocking antibody comprises Tocilizumab and/or Sarilumab.

111. The method of claim 108, wherein the treatment comprises an anti-inflammatory biologic and/or a steroid.

112. The method of claim 67, wherein the characterization of cellular molecular features and/or functional characteristics for the enriched rare circulating cells is associated with clinical data and laboratory results to identify one or more mechanisms of disease, biomarkers, and/or therapeutic targets related to changes in hematopoiesis.

113. The method of claim 112, wherein a subject is subsequently treated by administering a treatment directed to the one or more targets identified from the association of the cellular molecular features and/or functional characteristics for the enriched rare circulating cells with clinical data and laboratory results.

114. An enriched population of rare circulating cells from peripheral blood or a peripheral blood mononuclear cells (PBMC) cell fraction, prepared according to the method of any of claims 56-113.

115. The enriched population of rare circulating cells according to claim 114, for use in a method according to any of claims 1-55.
